(19) United States
(12) Patent Application Publication
(10) Pub. No.: US 2026/0232321 A1
(43) Pub. Date: Aug. 13, 2026

(54) CATHETER INSTRUMENT AND HEART FAILURE TREATMENT INSTRUMENT

(71) Applicant: PULNOVO MEDICAL (HK) LIMITED, Sheung Wan (HK)

(72) Inventors: Cynthia CHEN, Seattle, WA (US); Wen GU, Wilmington, DE (US); Shiche SUN, Shanghai (CN); Shiguan LE, Shanghai (CN); Qiuhua LI, Houston, TX (US)

(21) Appl. No.: 19/429,848

(22) Filed: Dec. 22, 2025

Related U.S. Application Data
(63) Continuation of application No. PCT/CN2025/112181, filed on Aug. 1, 2025.
(63) Continuation-in-part of application No. PCT/CN2024/100175, filed on Jun. 19, 2024.
(63) Continuation-in-part of application No. PCT/CN2024/101383, filed on Jun. 25, 2024.
(63) Continuation-in-part of application No. PCT/CN2024/101354, filed on Jun. 25, 2024.
(63) Continuation-in-part of application No. PCT/CN2024/101378, filed on Jun. 25, 2024.

(30) Foreign Application Priority Data
Jun. 27, 2023 (CN) 202310768924.5
Jun. 27, 2023 (CN) 202310773399.6
Jun. 27, 2023 (CN) 202310773544.0

(51) Int. Cl. A61B 17/12

(52) U.S. Cl. CPC A61B 17/12045; A61B 17/12109; A61B 17/12136; A61B 2017/1205

(57) ABSTRACT

A catheter instrument comprises: a catheter body having a device pass-through lumen, a balloon catheter pass-through lumen, a balloon inflation lumen, and a pressure monitoring lumen; a balloon sealingly coupled to the catheter body outer wall and connected to the balloon inflation lumen distal end; an attachment seat coupled to the catheter body proximal end and comprising a port for communication with the device pass-through lumen, a first side port for connection with the balloon catheter pass-through lumen, a second side port for connection with the balloon inflation lumen, and a third side port for connection with the pressure monitoring lumen or for passage of a diagnostic instrument. The catheter instrument further comprises a first side branch for inputting a balloon inflation medium in communication with the second side port; and a second side branch for inputting a pressure monitoring medium in communication with the third side port.

### CROSS-REFERENCE

This application is continuation of International Patent Application No. PCT/CN 2025/112181, filed Aug. 1, 2025; this application is also a continuation in part of International Patent Application No. PCT/CN2024/100175 (Catheter Instrument and Heart Failure Treatment Instrument), filed Jun. 19, 2024, which claims priority to Chinese Patent Application No. 202310773544.0, filed Jun. 27, 2023; this application is also a continuation in part of International Patent Application No. PCT/CN2024/101383 (Catheter Device and Heart Failure Treatment Instrument), filed Jun. 25, 2024, which claims priority to Chinese Patent Application No. 202310768924.5, filed Jun. 27, 2023; this application is also a continuation in part of International Patent Application No. PCT/CN2024/101354 (Balloon Sheath and Heart Failure Treatment Instrument), filed Jun. 25, 2024, which claims priority to Chinese Patent Application No. 202310768924.5, filed Jun. 27, 2023; and, this application is also a continuation in part of International Patent Application No. PCT/CN2024/101378 (Catheter Device and Heart Failure Treatment Instrument), filed Jun. 25, 2024, which claims priority to Chinese Patent Application No. 202310773399.6, filed Jun. 27, 2023, each of which applications are incorporated by reference in their entirety.

The subject matter of this application is related to that of PCT Application No. PCT/US 2023/068086 (Multi-Catheter Systems for Treating Heart Failure), filed Jun. 7, 2023, which claims priority to U.S. Provisional Patent Applications Nos. 63/349,975, filed Jun. 7, 2022, 63/397,289, filed Aug. 11, 2022, and 63/469,278, filed May 26, 2023, which are incorporated herein by reference in their entirety.

The subject matter of this application is related to that of PCT Application No. PCT/CN2024/099425 (Heart Failure Therapeutic Main Unit and Heart Failure Therapeutic Instrument), filed Jun. 14, 2024, which claims priority to Chinese Patent Application No. 202310721487.1, filed Jun. 16, 2023, which are incorporated herein by reference in their entirety.

### BACKGROUND

Heart failure is now the leading cause of death worldwide and its incidence is increasing year by year, effecting 56+ million patients worldwide. Heart failure is a condition in which the heart cannot pump enough blood to meet the body's metabolic needs due to a decrease in the pumping or filling capacity of the ventricles, resulting in inadequate perfusion of organs and tissues, as well as bruising of the lungs or the circulation. Heart failure is a syndrome of various heart diseases progressing to a severe stage and is often also referred to as congestive heart failure.

Acute heart failure is most common in acute left heart failure, which is a clinical syndrome that occurs due to acute myocardial damage or increased cardiac load, resulting in a sudden drop in acute cardiac output, elevated pressure in the pulmonary circulation, increased peripheral circulatory resistance, and subsequent congestion in the pulmonary circulation with acute pulmonary stasis, pulmonary edema, and may be accompanied by inadequate perfusion of tissues and organs and cardiogenic shock.

Further, studies have shown that the normal drainage function of the lymphatic system of most patients with acute heart failure will be significantly inhibited during the illness. The human body has a network of lymphatic vessels and lymph nodes throughout the body. All lymphatic vessels converge into the two largest lymphatic ducts in the body, namely the thoracic duct on the left and the right lymphatic duct on the right, and then enter the left and right subclavian veins respectively. The thoracic duct is the thickest and longest lymphatic vessel in the body, which collects lymph from the left upper and lower body, accounting for about 75% of the total lymph in the body. The right lymphatic duct collects lymph from the right upper body, accounting for about 25% of the total lymph in the body.

The function of lymph is to absorb various substances precipitated from blood vessels from the interstitial space and inject them back into the blood circulation through the lymphatic circulation. The flow of lymph is limited. In some cases, due to the increase in internal pressure caused by blood congestion in blood vessels, the amount of substances precipitated from blood vessels also increases significantly. At this time, the lymph cannot absorb the precipitated substances in time and transport them back to the blood circulation.

Chronic heart failure is categorized into right heart failure and left heart failure. In right heart failure, the ejection capacity of the right ventricle decreases and more blood remains in the right heart, resulting in increased pressure in the right heart and laborious flow of blood into the right heart. Since the blood flowing into the right heart is body circulation blood, there will be body circulation stagnation, and since the lower limbs are in the lower part of the body, the lower limbs stagnation is the most serious. When water passes from the blood vessels of the lower extremities into the surrounding tissues, edema of the lower extremities results. In left heart failure, the inflow of blood into the left heart from the pulmonary circulation leads to an increase in left atrial pressure and pulmonary venous pressure, which in turn leads to pulmonary bruising and pulmonary edema.

Acute decompensated heart failure (ADHF) is a sudden worsening of heart failure symptoms. To combat ADHF, a guideline-directed medical therapy is often implemented, utilizing ANRI/ACEI/ARB, β-blocker, MRAs, SGLT2is, Intravenous Diuretics, Vasodilators and Inotropics.

Current therapeutic strategies for acute heart failure prioritize oxygen supplementation, intravenous diuretics, and inotropic agents. If these measures fail, vasoactive drugs are administered to modulate vascular tone. In critical cases marked by persistent hypotension or cardiogenic shock, hemodynamic monitoring and advanced non-pharmacological interventions become necessary.

There are limitations to the treatment modalities in the relevant techniques; for example, some patients respond well to intravenous diuretic therapy, but others do not. This is because, when there is too much fluid in the body, most of the excess fluid is not actually present in the lumen of the blood vessels, but in the interstitial space around the cells, a tissue called the interstitium. In order to adequately reduce congestion, the excess fluid in the interstitium of the cells first needs to be moved into the blood vessels. In the healthy body, this function is accomplished through drainage by the lymphatic system, which actively drains the fluid into the large veins above the heart. However, in patients with acute heart failure, the pressure in the large veins can be very high, which slows or even prevents the flow of lymphatic fluid into the large veins, thus impeding the process of relieving congestion. Studies highlight that incomplete decongestion (residual fluid overload) is a strong predictor of rehospitalization and mortality. Approximately 50% of acute HF patients are discharged with unresolved fluid retention, leading to 25% readmission within one month and 50% within six months. Further, the current 5-year mortality rate is as high as 60%.

### SUMMARY

In accordance with the purposes of the disclosed devices and methods as embodied and broadly described herein, the disclosed subject matter relates to catheterization devices and heart failure, such balloon catheters, and methods of use thereof. Applicant has recognized that there is an unmet need for new methods and uses of heart catheter systems. Methods and uses of the present disclosure may be used to further catheter technology thus allowing for increased positive outcomes in the field.

According to an aspect, the present disclosure provides a method for treating a cardiovascular disease of a subject. In some embodiments, the method comprises: inserting at least a portion of a catheter system into a femoral vein of the subject; positioning a first balloon of the catheter system within the inferior vena cava of the subject at or near at least one renal vein; positioning a second balloon of the catheter system at or near a subclavian vein of the subject; and intermittently inflating the first balloon and the second balloon for a predetermined time period; wherein the intermittent inflation of the second balloon creates a low-pressure area at one or more of the left internal jugular vein or the thoracic duct of the subject, and wherein the intermittent inflation of the first balloon creates a low-pressure area at the renal vein junction of the subject, wherein the first balloon and second balloon are inflated simultaneously for a certain time of the pre-determined time period.

In some embodiments, the method further comprises: measuring the pressure at the subclavian vein of the subject using the catheter system; and adjusting the inflation of one or more of the first balloon or second balloon in response to the measured subclavian vein pressure.

In some embodiments, adjusting the inflation of the first balloon modulates the pressure at the renal vein junction, and wherein adjusting the inflation of the second balloon modulates the pressure at the one or more of the left internal jugular vein or thoracic duct.

In some embodiments, inflation of the second balloon stimulates a vagus nerve of the subject. In some embodiments, inflation of the first balloon stimulates a vagus nerve of the subject.

In some embodiments, inflating the first balloon comprises maintaining inflation of the first balloon for a portion of the pre-determined time before deflating the first balloon, wherein the pre-determined time is between from about 1 minute to about 30 minutes. In some embodiments, inflating the second balloon comprises maintaining inflation of the second balloon for a portion of the pre-determined time before deflating the second balloon, wherein the pre-determined time is between from about 1 minute to about 30 minute.

In some embodiments, the first balloon and second balloon are simultaneously inflated for 20% to 100% of the pre-determined time. In some embodiments, the first balloon and second balloon are simultaneously inflated for 25% of the pre-determined time. In some embodiments, the first balloon and second balloon are simultaneously inflated for 33% of the pre-determined time. In some embodiments, the first balloon and second balloon are simultaneously inflated for 66% of the pre-determined time.

In some embodiments, inflating the first balloon comprises maintaining inflation of the first balloon before deflating the first balloon. In some embodiments, inflating the second balloon comprises maintaining inflation of the first balloon before deflating the second balloon. In some embodiments, inflation of the first balloon and the second balloon is adjusted without external input.

In some embodiments, positioning the first balloon of the catheter system within the inferior vena cava comprises advancing a first catheter of the catheter system through vasculature of the subject over a guidewire. In some embodiments, positioning the second balloon at or near the subclavian vein of the subject comprises advancing a second catheter of the catheter system through vasculature of the subject over a guidewire.

In some embodiments, the method further comprises: inserting a catheter further comprising a third balloon into a lumen of the catheter system; positioning the third balloon of the catheter at or near the pulmonary artery; inflating the third balloon; and measuring a pulmonary artery pressure of the subject.

In some embodiments, positioning the third balloon of the catheter at or near the pulmonary artery comprises advancing the catheter through vasculature of the subject over a guidewire.

In some embodiments, the method further comprises monitoring at least one hemodynamic parameter of the subject with the catheter. In some embodiments, the at least one hemodynamic parameter is one or more of heart rate, blood pressure, stroke volume, cardiac output, or total peripheral resistance.

In some embodiments, the method further comprises monitoring pressure at a vein of the subject with the catheter system. In some embodiments, the method further comprises monitoring pressure at one or more of an inferior vena cava or femoral vein with the catheter system. In some embodiments, the method further comprises visualizing the at least the portion of the catheter system or with aid of at least one radiopaque marker of the catheter system. In some embodiments, the cardiovascular disease is heart failure or acute heart failure.

In some embodiments, the intermittent inflation of the first balloon promotes renal perfusion. In some embodiments, the promotion of renal perfusion is characterized by a urine output increase, glomerular filtration rate increase, measurement of renal blood flow, or renal resistance index reduction.

In some embodiments, the intermittent inflation of the first balloon promotes diuresis. In some embodiments, the promotion of diuresis is characterized by a urine output increase.

In some embodiments, the intermittent inflation of the first balloon reduces fluid retention. In some embodiments, the reduction of fluid retention is characterized by a subsiding of lower extremity edema or urine output increase.

In some embodiments, the intermittent inflation of the first balloon decreases venous return to the heart. In some embodiments, the venous return decrease to the heart is characterized by a pulmonary artery wedge pressure decrease or atrial and ventricular volume decreases.

In some embodiments, the intermittent inflation of the second balloon enhances venous drainage. In some embodiments, the enhancement of venous drainage is characterized by a renal perfusion improvement or an atrial and ventricular volume decrease.

In some embodiments, the intermittent inflation of the second balloon enhances lymphatic drainage. In some embodiments, the enhancement of lymphatic drainage is characterized by an atrial or ventricular volume decrease or a reduction of heart edema.

In some embodiments, the intermittent inflation of the second balloon relieves fluid overload. In some embodiments, the fluid overload relief is characterized by an alleviation of lung edema or controlled accumulation of fluid in the thoracic and abdominal cavity.

In some embodiments, the intermittent inflation of the second balloon relieves venous congestion. In some embodiments, the venous congestion relief is characterized by a pulmonary artery wedge pressure decrease or hepatic congestion alleviation.

In some embodiments, the intermittent inflation of the first and second balloon reduces pulmonary artery pressure. In some embodiments, the reduction of pulmonary artery pressure by the first balloon and the second balloon is characterized by a pressure decrease of at least 50% from pre-inflation pressure.

In an aspect, the present disclosure provides a method of treating a cardiovascular disease of a subject. In some embodiments, the method comprises: creating a low-pressure area at one or more of a left internal jugular vein or thoracic duct of the subject; and creating a low-pressure area at a renal vein of the subject, wherein creating the low-pressure area comprises intermittent occlusion of two or more veins of the subject by two or more balloons, wherein the two or more balloons are inflated simultaneously for a certain time of the pre-determined time period.

In some embodiments, creating the low-pressure area at the renal vein of the subject comprises intermittently occluding an inferior vena cava of the subject at or near the renal vein.

In some embodiments, intermittently occluding the inferior vena cava of the subject at or near the renal vein comprises advancing a catheter from a femoral vein of the subject near a renal vein and inflating a first balloon of the catheter at an inferior vena cava. In some embodiments, the intermittent occlusion of the two or more veins of the subject by the two or more balloons comprises inflating a first balloon and maintaining the inflation of the first balloon for a portion of the pre-determined time period before deflating the first balloon, wherein the portion of the pre-determined time is from about 1 minute to 30 minutes.

In some embodiments, creating the low-pressure area at the one or more of the left internal jugular vein or thoracic duct comprises intermittently occluding a subclavian vein of the subject.

In some embodiments, intermittently occluding the subclavian vein comprises advancing a catheter from a femoral vein of the subject and to subclavian vein and inflating a second balloon of the catheter at an inferior vena cava.

In some embodiments, the method further comprises maintaining inflation of the second balloon for a portion of the pre-determined time before deflating the second balloon, wherein the portion of the pre-determined time is from about 1 minute to about 30 minutes. In some embodiments, the first balloon and second balloon are simultaneously inflated for 20% to 100% of the pre-determined time. In some embodiments, the first balloon and second balloon are simultaneously inflated for 25% of the pre-determined time. In some embodiments, the first balloon and second balloon are simultaneously inflated for 33% of the pre-determined time. In some embodiments, the first balloon and second balloon are simultaneously inflated for 66% of the pre-determined time.

In some embodiments, creating the low-pressure area promotes renal perfusion or diuresis, reduces fluid retention, decreases venous return to the heart, enhances venous or lymphatic drainage, relieves fluid overload, or a combination thereof.

In some embodiments, creating a low-pressure area minimizes hemodynamic disturbance, achieves optimal therapeutic effects for acute heart failure, reduces pulmonary artery pressure, and demonstrates favorable acute and long-term safety. In some embodiments, the minimization of hemodynamic disturbance is characterized by maintenance of stable blood pressure, superior vena cava pressure, or inferior vena cava pressure. In some embodiments, the minimization of hemodynamic disturbance is characterized by a variation in pressure of less than 20 mmHg.

In some embodiments, optimal therapeutic effects is characterized by stabilization of hemodynamic disturbances. In some embodiments, favorable acute safety is demonstrated by hemodynamic stability. In some embodiments, favorable long-term safety is demonstrated by development of controllable vascular damage or thrombosis. In some embodiments, the cardiovascular disease is heart failure or acute heart failure.

In an aspect, the present disclosure provides a method of treating a cardiovascular disease of a subject. In some embodiments, the method comprises: intermittently occluding two or more veins to reduce venous blood backflow to the heart of the subject and reduce heart pumping burden, wherein the two or more veins are intermittently occluding synchronously and asynchronously at different points over a pre-determined time period.

In some embodiments, intermittently occluding the two or more veins comprises one or more of: intermittently occluding a subclavian vein of the subject, thereby creating a low-pressure area at a thoracic duct of the subject; or intermittently occluding an inferior vena cava of the subject, thereby creating a low-pressure area at a renal vein of the subject.

In some embodiments, intermittently occluding the subclavian vein comprises occluding the subclavian vein for from about 1 minute to about 30 minutes. In some embodiments, intermittently occluding the inferior vena cava comprises occluding the inferior vena cava for about 1 minute to about 30 minutes. In some embodiments, the inferior vena cava and subclavian vein are synchronously occluded for 20% to 100% of the pre-determined time. In some embodiments, the inferior vena cava and subclavian vein are synchronously occluded for 25% of the pre-determined time. In some embodiments, the inferior vena cava and subclavian vein are synchronously occluded 33% of the pre-determined time. In some embodiments, the inferior vena cava and subclavian vein are synchronously occluded for 66% of the pre-determined time.

In some embodiments, intermittently occluding the two or more veins comprises expanding two or more balloons of a catheter system at or near the two or more veins. In some embodiments, the expansion of the two or more balloons is configured to intermittently occlude a portion of the two or more veins. In some embodiments, the expansion of the two or more balloons intermittently occludes 50% to 100% of the two or more veins. In some embodiments, the expansion of the two or more balloons intermittently occludes 70% to 100% of the two or more veins.

In some embodiments, intermittently occluding the two or more veins stimulates a vagus nerve of the subject.

In some embodiments, intermittently occluding the two or more veins one or more of minimizes hemodynamic disturbance, achieves optimal therapeutic effects for acute heart failure, and demonstrates favorable acute and long-term safety. In some embodiments, the cardiovascular disease is heart failure or acute heart failure.

In some embodiments, intermittent occlusion of the two or more veins improves one or more of cardiac output or ventricular ejection fraction. In some embodiments, cardiac output increase is characterized by an overall mL/min increase over at least 8 hours. In some embodiments, intermittent occlusion of the two or more veins allows for controllable vascular results at least 30 days from treatment. In some embodiments, controllable vascular results is characterized by controllable vascular damage or thrombosis.

According to an aspect of the present disclosure, there is provided a catheter instrument comprising a catheter body having a device pass-through lumen, a balloon catheter pass-through lumen, a balloon inflation lumen, and a pressure monitoring lumen that are not in communication with each other; a balloon that is sealingly connected to an outer wall of the catheter body and connected to a distal end of the balloon inflation lumen; an attachment, or coupling, seat that is connected to the proximal end of the catheter body and comprises a port in communication with the device pass-through lumen, a first side port in communication with the balloon catheter through the lumen, a first side port in communication with the balloon inflation lumen, a second side port in communication with the balloon inflation lumen, and a third side port in communication with the pressure monitoring lumen, wherein the port is for passage of a diagnostic and therapeutic instrument and the first side port is for passage of a balloon catheter; a first side branch for input of balloon inflation medium in communication with the second side port; and a second side branch for input of a pressure monitoring medium in communication with the third side port.

In some embodiments, the catheter body is provided with at least one through-hole that connects the balloon pressurized lumen to the balloon, the balloon being a compliant wall-like balloon that fits against an outer wall of the catheter body in a contracted state.

In some embodiments, the material of the balloon includes at least one of silicone, latex, and polyurethane.

In some embodiments, the instrument pass-through lumen, the balloon catheter pass-through lumen, the balloon inflation lumen, and the pressure monitoring lumen are each circularly perforated, wherein the center of a cross-section of the instrument pass-through lumen, the center of a cross-section of the catheter body, and the center of a cross-section of the balloon catheter pass-through lumen are disposed sequentially along a first straight line direction; the center of a cross-section of the balloon inflation lumen and the center of a cross-section of the pressure monitoring lumen are disposed sequentially along a center of the cross-section of the balloon pressure-filling lumen and the center of the cross-section of the pressure-monitoring lumen are disposed sequentially along a second linear direction orthogonal to the first linear direction.

In some embodiments, the aperture R₁ of the lumen through which the device passes, the aperture R₂ of the lumen through which the balloon catheter passes, the aperture R₃ of the lumen through which the balloon is inflated, and the aperture R₄ of the lumen through which the pressure is monitored, satisfy the following: R₁>R₂>R₃=R₄.

In some embodiments, the catheter body includes a main body portion and a tip portion coupled to a distal end of the main body portion, wherein the instrument leads to a side of the tip portion through the lumen and a distal end of the pressure monitoring lumen, and the balloon catheter leads to an end face of the tip portion through the distal end of the lumen.

In some embodiments, the catheter instrument further comprises: a first developing element disposed on an outer wall of the body portion; and/or a second developing element disposed on a side of the tip portion.

In some embodiments, the catheterization device further comprises: a first gland disposed at the port, and a first hemostatic valve disposed within the port and secured by the first gland; and a second gland disposed at the first side port, and a second hemostatic valve disposed within the first side port and secured by the second gland.

In some embodiments, the first hemostatic valve is a radial compression hemostatic valve and the second hemostatic valve is a cross-cut hemostatic valve.

In some embodiments, a luer fitting tee is provided at the proximal end of each of the first side branch and the second side branch.

In some embodiments, the diagnostic device includes an examination catheter, a floating catheter, or an ablation catheter.

According to an aspect of the present disclosure, there is provided a heart failure treatment device comprising a catheterization device of the preceding aspect.

According to one or more embodiments of the present disclosure, the veins may be blocked intermittently using the balloon of the catheterization device and the balloon of the balloon catheter passing through the catheterization device, thereby reducing the return of venous blood to the heart, decreasing the preload on the heart, and allowing for a reduction in ventricular wall stress.

According to one aspect of the present disclosure, a catheter device is provided, comprising: a multi-lumen tube, having a first lumen, a second lumen, a third lumen and a fourth lumen which are not connected to each other, and a tube wall of the multi-lumen tube is provided with a first through-hole, a second through-hole and a third through-hole in sequence in a direction away from a distal end wherein the first lumen runs through the multi-lumen tube and is used for a guide wire to pass through; the distal end of the second lumen is closed, and the second lumen is connected to the second through-hole, and the second lumen is used for transporting lymph or contrast fluid through the second through-hole; the third lumen The distal end is closed, and the third lumen extends to the proximal end of the multi-lumen tube, the third lumen is connected to the first through-hole, and is used to transport a pressurized medium through the first through-hole; the distal end of the fourth lumen is closed, and the fourth lumen extends to the proximal end of the multi-lumen tube, the fourth lumen is connected to the third through-hole, and is used to transport a pressurized medium through the third through-hole; a first balloon is sealed and connected to the outer wall of the multi-lumen tube and is connected to the third lumen through the first through-hole; and a second balloon is sealed and connected to the outer wall of the multi-lumen tube and is connected to the fourth lumen through the third through-hole.

In some embodiments, the multi-lumen tube also has a fifth lumen for draining blood that is not connected to the first lumen, the second lumen, the third lumen, and the fourth lumen, and the tube wall of the multi-lumen tube is also provided with a fourth through-hole and a fifth through-hole, wherein the fourth through-hole, the first through-hole, the second through-hole, the third through-hole, and the fifth through-hole are arranged in sequence in a direction away from the distal end of the multi-lumen tube, the distal end and the proximal end of the fifth lumen are closed, and the fifth lumen is connected to the fourth through-hole and the fifth through-hole.

In some embodiments, the catheter device also includes: a connecting seat, which is connected to the proximal end of the multi-lumen tube and has a first connector connected to the first lumen, a second connector connected to the second lumen, a third connector connected to the third lumen, and a fourth connector connected to the fourth lumen.

In some embodiments, the proximal end of the second lumen is closed, and the catheter device further includes: an axial flow pump disposed in the second lumen; and a reflux tube connected to the second lumen, for transporting the lymph fluid in the second lumen to the venous blood vessel under the action of the axial flow pump.

In some embodiments, the second lumen, the third lumen, the fourth lumen and the fifth lumen are distributed around the circumference of the first lumen, and the third lumen and the fourth lumen are arranged opposite to each other.

In some embodiments, a blocking material is used to block the distal end of the fifth lumen and the distal end of the multi-lumen tube, and a blocking material is used to block the proximal end of the fifth lumen and the proximal end of the multi-lumen tube.

In some embodiments, the number of the first through-hole and the number of the third through-hole are plural.

In some embodiments, the catheter device further includes: a first imaging element disposed on the periphery of the multi-lumen tube and located in the first balloon; and a second imaging element disposed on the periphery of the multi-lumen tube and located in the second balloon.

In some embodiments, the distal end of the multi-lumen tube is tapered.

In some embodiments, the materials of the first balloon and the second balloon respectively include at least one of polyamide, polyurethane, medical latex, medical silicone, polyamide polyether block copolymer, and polyethylene.

According to another aspect of the present disclosure, a heart failure treatment device is provided, comprising the catheter device of any one of the aforementioned embodiments.

According to one or more embodiments of the present disclosure, the use of the above-mentioned catheter device in the treatment of heart failure can enhance the absorption and drainage functions of the lymphatic system, thereby more efficiently transferring the stagnant interstitial fluid, effectively alleviating fluid retention, and improving the effect of heart failure treatment.

These and other aspects of the disclosure will be apparent from and elucidated with reference to the embodiments described hereinafter.

According to an aspect of the present disclosure, there is provided a balloon sheath comprising: a main sheath having a main lumen, a secondary lumen, a first pressure-filling lumen, and a second pressure-filling lumen that are not in communication with each other, the main lumen being used for guiding a diagnostic and therapeutic instrument to be threaded out, and the secondary lumen being used for guiding a balloon catheter to be threaded out; a first balloon sealedly coupled with an outer wall of the main sheath and connected to the distal end of the first pressure-filling lumen and the second pressure-filling lumen; a main connection seat coupled to the proximal end of the main sheath and includes a first port communicating with the main lumen, a second port communicating with the secondary lumen, a first side port communicating with the main lumen, and a second side port communicating with the first and second insufflating lumens, the first port being used for passage of diagnostic and therapeutic instruments; a secondary sheath communicating with the second port; a secondary connector connecting proximally to the secondary sheath and including a third port and third side port communicating with the secondary sheath; a third port used for passage of balloons; and a primary balloon sealed to the outer wall of the primary sheath and connected to the distal end of the first and second insufflating lumens. side port, the third port for passage of a balloon catheter; and a first side branch tube in communication with the first side port, a second side branch tube in communication with the second side port for input of a pressure-filled medium, and a third side branch tube in communication with the third side port.

In some embodiments, the main sheath lumen and the secondary lumen are each in the shape of a circular hole, and the main sheath lumen has an aperture that is larger than the aperture of the secondary lumen; the first pressurized lumen and the second pressurized lumen are disposed on both sides of the main sheath lumen and the secondary lumen.

In some embodiments, the main sheath is provided with at least one through-hole connecting the first pressure-filled lumen and the second pressure-filled lumen to a first balloon, the first balloon being a compliant walled balloon that fits against an outer wall of the main sheath in a contracted state.

In some embodiments, the material of the first balloon includes at least one of silicone, latex, polyurethane, and polyurethane.

In some embodiments, the primary sheath includes a first tip portion adjacent to a distal end thereof, a distal perforation of the primary lumen is exposed to an end surface of the first tip portion, and a distal perforation of the secondary lumen is exposed to a side surface of the first tip portion.

In some embodiments, a luer fitting tee is provided at the proximal end of each of the first lateral branch, the second lateral branch, and the third lateral branch.

In some embodiments, the balloon sheath further comprises: a primary hemostatic valve disposed within the primary connection seat; and a secondary hemostatic valve disposed within the secondary connection seat.

According to one aspect of the present disclosure, there is provided a heart failure treatment device comprising the balloon sheath tube of the preceding aspect.

In some embodiments, the heart failure therapeutic device further comprises: a balloon catheter nested assembled with the secondary sheath and the primary sheath, the balloon catheter comprising: a multi-lumen tube having a guidewire entry lumen, a pressure-filled media delivery lumen, and a contrast delivery lumen that are not in communication with each other, and a contrast delivery port that is exposed in an outer wall of the multi-lumen tube and that is in communication with a distal end of the contrast delivery lumen; a single-lumen tube in communication with the distal end of the guidewire entry lumen; a second balloon in communication with the distal end of the guidewire entry lumen; and a second balloon in communication with the secondary sheath. connected to the distal end of the guidewire entry lumen; a second balloon, sealed to the outer wall of the multi-lumen tube and the outer wall of the single-lumen tube and connected to the distal end of the pressurized medium delivery lumen; and a coupling seat, connected to the proximal end of the multi-lumen tube and comprising a first connector connected to the guidewire entry lumen, a second connector connected to the pressurized medium delivery lumen, and a third connector connected to the contrast medium delivery lumen, the first connector being used for guidewire entry, the second connector for inputting the pressure-filled medium, the second connector for inputting the contrast agent, and the third connector for inputting the contrast agent.

In some embodiments, the guidewire insertion lumen is circularly perforated and its center axis coincides with the center axis of the multi-lumen tube; the pressure-filled medium delivery lumen and the contrast agent delivery lumen are each arcuate perforated around the guidewire insertion lumen.

In some embodiments, the second balloon is a non-compliant balloon, and the material of the second balloon includes at least one of polyamide, polyether block polyamide, and polyethylene.

In some embodiments, the contrast agent delivery aperture is located on a side of the second balloon away from the single lumen tube.

In some embodiments, the single lumen tube includes a second tip portion adjacent to a distal end thereof.

In some embodiments, the balloon catheter further comprises a developer element disposed in an outer wall of the single lumen tube.

In some embodiments, the balloon catheter further comprises a reinforcing sleeve, sleeved over a connection of the multi-lumen tube to the connection seat and sealing the multi-lumen tube to the connection seat.

According to one or more embodiments of the present disclosure, on the one hand, the first balloon of the balloon sheath tube and the second balloon of the balloon catheter passing through the balloon sheath tube can be utilized to intermittently seal the vein, thereby reducing the return of venous blood to the heart, lowering the preload of the heart, and making the ventricular wall less stressful; on the other hand, it is possible to make the first balloon of the balloon sheath tube and the second balloon of the balloon catheter passing through the balloon sheath tube synchronized with the cardiac rhythm, so as to synchronize filling and deflating. On the other hand, the first balloon of the balloon sheath and the second balloon of the balloon catheter passing through the balloon sheath are synchronized with the cardiac rhythm to inflate and deflate, thus producing a double hemodynamic effect, increasing diastolic blood pressure and coronary perfusion, and decreasing the cardiac afterload to improve left ventricular ejection. The main lumen of the balloon sheath tube can also be passed into a diagnostic device for relevant testing and treatment during or before and after treatment. Thus, the balloon sheath tube of the embodiments of the present disclosure can be used for treating acute congestive heart failure and can achieve a good prognosis.

According to one aspect of the present disclosure, a catheter device is provided, comprising an inner balloon catheter and an outer balloon catheter that are nested and assembled, wherein:

The inner balloon catheter comprises: a first multi-lumen tube, having a first lumen and a second lumen which are not communicated with each other; a first single-lumen tube, connected to the distal end of the first lumen; a first balloon, sealedly connected to the outer wall of the first multi-lumen tube and the outer wall of the first single-lumen tube and connected to the distal end of the second lumen; and a first hub, connected to the proximal end of the first multi-lumen tube and comprising a first connector connected to the first lumen and a second connector connected to the second lumen, wherein the first connector, the first lumen and the first single-lumen tube are used for the guide wire to penetrate, and the second connector and the second lumen are used for conveying a pressurized medium;

The outer balloon catheter comprises: a second multi-lumen tube, having a third lumen and a fourth lumen that are not connected to each other; a second single-lumen tube, connected to the distal end of the third lumen; a second balloon, sealedly connected to the outer wall of the second multi-lumen tube and the outer wall of the second single-lumen tube and connected to the distal end of the fourth lumen; and a second hub, connected to the proximal end of the second multi-lumen tube and comprising a third connector connected to the third lumen, a fourth connector connected to the fourth lumen, and a locking structure arranged at the proximal end of the third connector, wherein the third connector, the third lumen, and the second single-lumen tube are used for the inner balloon catheter to penetrate, the fourth connector and the fourth lumen are used to transport a pressurized medium, and the locking structure is used to lock the relative sliding position of the inner balloon catheter in the outer balloon catheter.

In some embodiments, the locking structure includes: an external threaded section, disposed at the proximal end of the third connector; a gland, having an internal threaded section connected to the external threaded section; and a hemostatic valve, disposed on the inner side of the external threaded section and having a through hole for the inner balloon catheter to pass through, and when the gland is tightened, the hemostatic valve presses the inner balloon catheter to lock its relative sliding position in the outer balloon catheter.

In some embodiments, the first balloon and the second balloon are double-layer balloons respectively, and the double-layer balloons include an outer layer material and an inner layer material bonded to each other, wherein the hardness of the outer layer material is greater than the hardness of the inner layer material.

In some embodiments, the outer layer material includes at least one of polyamide, polyether block polyamide, and polyethylene terephthalate; the inner layer material includes at least one of polyether block polyamide, polyurethane, thermoplastic elastomer, silicone, and latex.

In some embodiments, the first lumen is in the shape of a circular hole, and the second lumen is in the shape of a crescent hole, wherein the central axis of the first lumen is eccentrically disposed relative to the central axis of the first multi-lumen tube; the third lumen is in the shape of a circular hole, and the fourth lumen is in the shape of a crescent hole, wherein the central axis of the third lumen is eccentrically disposed relative to the central axis of the second multi-lumen tube.

In some embodiments, the material of the first multi-lumen tube and the second multi-lumen tube includes at least one of polyamide, polyether block polyamide, polyurethane, polyethylene, and polypropylene.

In some embodiments, the first single-lumen tube and the second single-lumen tube each include: a body portion; and a tip portion connected to a distal end of the body portion.

In some embodiments, the material of the body portion includes at least one of polyamide and polyether block polyamide; the material of the tip portion includes at least one of polyurethane, polyurethane, polyether block polyamide, and silicone.

In some embodiments, the inner balloon catheter further comprises: a reinforcing sleeve, which is sleeved at the connection between the first multi-lumen tube and the first hub and seals the first multi-lumen tube and the first hub.

In some embodiments, the material of the reinforcing sleeve includes one or more of polyamide, acrylonitrile-butadiene-styrene terpolymer, polyolefin, polyether block polyamide, and metal oxide.

In some embodiments, the inner balloon catheter further includes: a first developing element disposed on the outer wall of the first single-lumen tube and located in the first balloon; the outer balloon catheter further includes: a second developing element disposed on the outer wall of the second single-lumen tube and located in the second balloon.

In some embodiments, the material of the first developing element and the second developing element includes at least one of gold, platinum, iridium, tantalum, and tungsten.

In some embodiments, the material of the first hub and the second hub includes at least one of polyamide, polycarbonate, and polyoxymethylene.

In some embodiments, the outer balloon catheter is used to access the inferior vena cava and the inner balloon catheter is used to access the subclavian vein via the outer balloon catheter.

According to another aspect of the present disclosure, a heart failure treatment device is provided, comprising the catheter device of the aforementioned embodiment.

According to one or more embodiments of the present disclosure, the double balloons of the catheter device can be used to intermittently block the veins, thereby reducing the venous blood backflow to the heart, reducing the precardiac load, and reducing the stress on the ventricular wall. This can be used to treat acute congestive heart failure and achieve a good prognosis.

These and other aspects of the disclosure will be apparent from and elucidated with reference to the embodiments described hereinafter.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 is a schematic diagram of the main view structure of a catheterization device of some embodiments of the present disclosure.

FIG. 2 is a schematic diagram of the three-dimensional structure of a catheterization instrument of some embodiments of the present disclosure.

FIG. 3 is a schematic structural diagram of a catheter device according to some embodiments of the present disclosure.

FIG. 4 is a schematic diagram of the structure of a catheter device according to some embodiments of the present disclosure.

FIG. 5 is a schematic diagram of a structure of a heart failure treatment device of some embodiments of the present disclosure.

FIG. 6 is a schematic structural diagram of a balloon sheath tube of some embodiments of the present disclosure.

FIG. 7 is a schematic structural diagram of a balloon catheter of some embodiments of the present disclosure.

FIG. 8 is a schematic diagram of a three-dimensional structure of a catheter device according to some embodiments of the present disclosure.

FIG. 9 is a front view of the structure of a catheter device according to some embodiments of the present disclosure.

FIG. 10 is a schematic diagram of a disassembled structure of a second hub of a catheter device according to some embodiments of the present disclosure.

FIG. 11 is a schematic diagram of a cross-sectional structure of a first balloon or a second balloon of a catheter device according to some embodiments of the present disclosure.

FIG. 12 is a schematic depicting a balloon catheter system for the treatment of heart failure, according to some embodiments.

FIGS. 13A-13D are a series of section view illustrations depicting a method for using the balloon catheter systems described herein to treat heart failure, according to some embodiments.

FIGS. 14A-14D are a series of section view illustrations depicting a method for advancing a pulmonary artery catheter to the third target location at the pulmonary artery.

FIGS. 15A-15B are visualizations of the pulmonary artery pressure (FIG. 15A) and subclavian vein pressure (FIG. 15B) as a result of simultaneous occlusion of the infra-renal inferior vena cava and subclavian vein.

FIGS. 16A-16C are visualizations of systolic blood pressure as a result of partial occlusion of the subclavian vein and inferior vena cava.

FIG. 17 is a visualization of hemodynamic parameters before and after partial occlusion of the subclavian vein and inferior vena cava.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Embodiments of the present disclosure provide a catheterization device and a heart failure treatment device that can be used to treat acute congestive heart failure and can achieve a good prognosis.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Unless otherwise defined, all technical terms used herein have the same meaning as commonly understood by one of ordinary skill in the art to which this disclosure belongs. As used herein, the singular forms “a,” “an,” and “the” include plural references unless the context clearly indicates otherwise. Any reference to “or” herein is intended to encompass “and/or” unless otherwise stated. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper”, “center”, “longitudinal”, “transverse”, “length”, “width”, “thickness”, “top”, “bottom”, “bottom”, “thickness”, “length”, “width”, “thickness”, “top”, “bottom”, “front”, “back”, “left”, “right”, “top”, “bottom”, “inside”, “outer”, “clockwise”, “counterclockwise”, “axial”, “radial” , “circumferential”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first”, “second”, “third”, etc. may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

For the purposes of this disclosure, unless otherwise expressly provided and limited, the terms “mounted”, “connected”, “connected”, “fixed”, and the like shall be broadly construed. “ and the like are to be broadly construed, for example, as a fixed connection, a removable connection, or an integral part; a mechanical connection, an electrical connection, or a communication; a direct connection, an indirect connection through an intermediate medium, a connection within two elements, or an interactive relationship between two elements. For those of ordinary skill in the art, the specific meaning of the above terms in the present disclosure may be understood on a case-by-case basis.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The present disclosure relates to the technical field of medical devices and, in particular, to a catheterization devices, or instruments, and a heart failure treatment device.

Reducing ventricular wall stress by decreasing cardiac preload is fundamental to the treatment of acute congestive heart failure. The Frank-Starling mechanism (a compensatory mechanism in heart failure) suggests that preload is a major determinant of cardiac output and further suggests that in patients with systolic heart failure in the presence of volume overload, reduction of volume overload may improve, or at least not decrease, cardiac output. The results of several current studies suggest that elevated cardiac filling pressures directly affect the short-and long-term prognosis of patients with heart failure.

Among the current treatments for heart failure, cardiac preload is often reduced by diuretic therapy and vasodilation. Methods to reduce cardiac congestion through specialized devices include accelerated water separation, hemodialysis, and some innovative methods such as pumps within the descending aorta. However, few devices specifically designed to reduce cardiac preload are currently available due to concerns about the potential to reduce cardiac output and systemic blood pressure.

The intermittent balloon filling and blocking of the superior and inferior vena cava blood can effectively reduce the heart's pumping pressure and can enhance the excess fluid in the interstitial cells to enter the lymphatic fluid and return to the blood vessels, thereby reducing fluid retention and improving the patient's postoperative cure. In the intermittent balloon filling and blocking of the superior and inferior vena cava blood program, the heart failure treatment device fills the balloon catheter with fluid, pressurizes it, and extracts and releases it according to the set intervals, thereby achieving intermittent balloon blocking of the superior and inferior vena cava, achieving the purpose of reducing the heart's pumping pressure and reducing fluid retention during heart failure.

Further, intermittent occlusion of the inferior vena cava below the renal veins allows for creation of a low-pressure zone at the renal vein junction. The low-pressure zone promotes renal perfusion and diuresis, reduces fluid retention, and decreases venous return to the heart. Additionally, intermittent occlusion of the subclavian vein allows for creation of a low-pressure zone near the thoracic duct. The low-pressure zone enhances venous and lymphatic drainage, further reliving fluid overload and venous congestion.

In some examples, intermittent inflation of a balloon may promote renal perfusion. In some examples, intermittent inflation of the first balloon may promote renal perfusion. In some examples, intermittent inflation of the second balloon may promote renal perfusion. In some examples, promotion of renal perfusion may be characterized by an increase in urine output, an increase in glomerular filtration rate, measurement of renal blood flow, or a reduction in renal resistance index. In some examples, the renal blood flow may be measured by isotope and radionuclide methods. In some examples, renal resistance index may be in the kidney and examined by ultrasound.

In some examples, intermittent inflation of a balloon may promote diuresis. In some examples, intermittent inflation of the first balloon may promote diuresis. In some examples, intermittent inflation of the second balloon may promote diuresis. In some examples, promotion of diuresis may be characterized by an increase in urine output.

In some examples, intermittent inflation of a balloon may reduce fluid retention. In some examples, intermittent inflation of the first balloon may reduce fluid retention. In some examples, intermittent inflation of the second balloon may reduce fluid retention. In some examples, reduction of fluid retention may be characterized by subsiding of lower extremity edema or increase in urine output.

In some examples, intermittent inflation of a balloon may decrease venous return to the heart. In some examples, intermittent inflation of the first balloon may decrease venous return to the heart. In some examples, intermittent inflation of the second balloon may decrease venous return to the heart. In some examples, venous return decrease to the heart may be characterized by a pulmonary artery wedge pressure decrease or atrial and ventricular volume decreases.

In some examples, intermittent inflation of a balloon may enhance venous drainage. In some examples, intermittent inflation of the first balloon may enhance venous drainage. In some examples, intermittent inflation of the second balloon may enhance venous drainage. In some examples, enhancement of venous drainage may be characterized by a renal perfusion improvement or an atrial and ventricular volume decrease under ultrasound examination.

In some examples, intermittent inflation of a balloon may enhance lymphatic drainage. In some examples, intermittent inflation of the first balloon may enhance lymphatic drainage. In some examples, intermittent inflation of the second balloon may enhance lymphatic drainage. In some examples, enhancement of lymphatic drainage may be characterized by atrial or ventricular volume decrease under ultrasound examination or a reduction of heart edema shown by cardiac MRI.

In some examples, intermittent inflation of a balloon may relieve fluid overload. In some examples, intermittent inflation of the first balloon may relieve fluid overload. In some examples, intermittent inflation of the second balloon may relieve fluid overload. In some examples, fluid overload relief may be characterized by an alleviation of lung edema under CT/X-ray or controlled accumulation of fluid in the thoracic and abdominal cavity under ultrasound.

In some examples, intermittent inflation of a balloon may relieve venous congestion. In some examples, intermittent inflation of the first balloon may relieve venous congestion. In some examples, intermittent inflation of the second balloon may relieve venous congestion. In some examples, venous congestion relief may be characterized by a pulmonary artery wedge pressure decrease or hepatic congestion alleviation under ultrasound.

In some examples, intermittent inflation of a balloon may reduce pulmonary artery pressure. In some examples, intermittent inflation of the first balloon may reduce pulmonary artery pressure. In some examples, intermittent inflation of the second balloon may reduce pulmonary artery pressure. In some examples, intermittent inflation of the first balloon and second balloon may reduce pulmonary artery pressure. In some examples, pulmonary artery pressure reduction may be characterized by a pressure decrease from pre-inflation pressure. In some examples, the pressure decrease may be from about 10% to about 90% from pre-inflation pressure. In some examples, the pressure decrease may be from about 10% to about 30% from pre-inflation pressure. In some examples, the pressure decrease may be from about 30% to about 60% from pre-inflation pressure. In some examples, the pressure decrease may be from about 60% to about 90% from pre-inflation pressure. In some examples, the pressure decrease may be at least about 50% from pre-inflation pressure.

Embodiments of the present disclosure provide a catheterization device and a heart failure treatment device that can be used to treat acute congestive heart failure and can achieve a good prognosis.

In some examples, a catheter instrument may be present. In some examples, the catheter instrument may be comprising a catheter body having a device pass-through lumen, a balloon catheter pass-through lumen, a balloon inflation lumen, and a pressure monitoring lumen that are not in communication with each other; a balloon that is sealingly connected to an outer wall of the catheter body and connected to a distal end of the balloon inflation lumen; an attachment, or coupling, seat that is connected to the proximal end of the catheter body and comprises a port in communication with the device pass-through lumen, a first side port in communication with the balloon catheter through the lumen, a first side port in communication with the balloon inflation lumen, a second side port in communication with the balloon inflation lumen, and a third side port in communication with the pressure monitoring lumen, wherein the port is for passage of a diagnostic and therapeutic instrument and the first side port is for passage of a balloon catheter; a first side branch for input of balloon inflation medium in communication with the second side port; and a second side branch for input of a pressure monitoring medium in communication with the third side port.

In some examples, the catheter instrument may comprise a catheter body. In some examples, the catheter body may have a distal and proximal end. In some examples, the catheter body may comprise an inner wall and an outer wall. In some examples, the catheter body may comprise a lumen. In some examples, the catheter body may comprise a device pass-through lumen. In some examples, the catheter body may comprise a balloon catheter pass-through lumen. In some examples, the catheter body may comprise a balloon inflation lumen. In some examples, the catheter body may comprise a pressure monitoring lumen. In some examples, each lumen may have a distal and proximal end. In some examples, the catheter instrument may comprise a balloon. In some examples, the catheter instrument may comprise an attachment, or coupling, seat. In some examples, the catheter instrument may comprise a port, wherein the port may be co-planar or be a side port. In some examples, the catheter instrument may comprise a side, or lateral, branch. In some examples, the catheter body may be a tube. In some examples, the catheter body may be a tube configured to house a lumen. In some examples, the catheter body may be a tube configured to house multiple lumens, or a multi-lumen tube.

In embodiments of the present disclosure, the end of the catheterization device or component thereof that extends closer to the operator in the direction of extension is defined as the “proximal end”, and similarly, the end of the catheterization device or component thereof that extends further away from the operator in the direction of extension is defined as the “distal end”. A “side port” is defined as an orifice provided on the side surface of the component, and a “port” is defined as an orifice provided on the end surface of the component.

In some examples, the catheter instrument may be configured to provide access to diagnostic instruments. In some examples, the catheter instrument may be configured to provide access to therapeutic instruments. In some examples, the instrument access may be through the pass-through lumen. In some examples, the pass-through lumen may be threaded. In some examples, the catheter instrument may be configured to provide access for a catheter, such as a balloon, inspection, floating, ablation, or any acceptable catheter. In some examples, as the catheter instrument is being positioned, the balloon may be guided into the subject. In some examples, as the catheter instrument is being positioned, the instrument may be guided into the subject. In some examples, the instrument or balloon guiding may be aided by an associated guide member.

In some examples, the catheter instrument may be configured to provide access for balloon catheters. In some examples, the balloon catheter access may be through the balloon catheter lumen. In some examples, the balloon catheter lumen may be threaded into a side port. In some examples, the catheter instrument may be configured to provide access for instruments to seal a blood vessel. In some examples, the catheter instrument may be configured to seal the blood vessel by filling the balloon. In some examples, the balloon may be configured to occlude the blood vessel using the balloon of the catheter device or the balloon from the balloon catheter. In some examples, the catheter instrument balloon and balloon catheter balloon may intermittently pass through a vessel to seal the vessel. In some examples, the balloons may be configured to reduce the return of blood to the heart. In some examples, the reduction of blood return may reduce the preload on the heart. In some examples, the reduction of blood may reduce ventricular wall stress.

In some examples, the catheter instrument may be configured to seal the blood by filling the balloon with the aid of a pressure pump. In some examples, the pressure pump may be an external pressure pump. In some examples, the pressure pump may be an internal pressure pump. In some examples, the pressure pump may fill the balloon through the balloon filling lumen with a pressure-filling medium through a first side branch. In some examples, the catheter instrument may be configured to monitor intravascular pressure. In some examples, the intravascular pressure may be monitored by a pressure sensor. In some examples, the intravascular pressure may be monitored by an external pressure sensor. In some examples, the intravascular pressure may use the pressure-monitoring lumen. In some examples, the pressure monitoring may fill the balloon through the balloon monitoring lumen with a pressure-monitoring medium through a second side branch. In some examples, the pressure monitoring may be directed to the distal end of the catheter instrument and to the blood vessel. In some examples, the pressure monitoring medium may be any acceptable medium for the treatment plan, such as heparin saline. In some examples, pressure monitoring may be achieved before, during, and after the procedure.

In some examples, the catheter may have a size. In some examples, the catheter size may be in French size, or by outer diameter of the catheter body, or tube. In some examples, the catheter size may be from 3 French (F) to about 34 F. In some examples, the catheter size may be from about 3 F to about 14 F. In some examples, the catheter size may be from about 14 F to about 24 F. In some examples, the catheter size may be from about 24 F to about 34 F. In some examples, the catheter size may be about 5F, 6F, 8F, or 18F.

In some examples, the catheter body may have an effective length. In some examples, the catheter body may have an effective length from around 200 mm (millimeter) to about 2000 mm. In some examples, the catheter body may have an effective length from about 200 mm to about 800 mm. In some examples, the catheter body may have an effective length from about 800 mm to about 1400 mm. In some examples, the catheter body may have an effective length from about 1400 mm to about 2000 mm. In some examples, the catheter body may have an effective length of about 600 mm. In some examples, the catheter body may have an effective length of about 1400 mm.

In some examples, the catheter body may comprise an instrument material. In some examples, the catheter tip portion and body portion may comprise an instrument material. In some examples, the instrument material may comprise a metal. In some examples, the instrument material may comprise a non-metal. In some examples, the instrument material may comprise a polymer. In some examples, the instrument material may comprise plastic. In some examples, the instrument material may comprise polyamide. In some examples, the instrument material may comprise polyether block polyamide. In some examples, the instrument material may comprise acrylonitrile-butadiene-styrene terpolymer. In some examples, the instrument material may comprise polyethylene glycol p-toluene dicarboxylate. In some examples, the instrument material may be polyurethane. In some examples, the instrument material may be polyimide ester. In some examples, the instrument material may be silicone. In some examples, the instrument material may be selected to reduce resistance with the blood vessel for smoother entry.

In some examples, each body component (catheter body, tip portion, body portion) may comprise the same instrument material. In some examples, each body component may comprise a different instrument material. In some examples, the body component instrument material may be selected due to their flexibility. In some examples, the body component instrument material may be selected due to their flexibility in comparison to the material of another component. In some examples, the body components may be coupled. In some examples, the body components may be coupled by glue, welding, solder, or any other acceptable coupling agent.

In some examples, the catheter instrument may comprise a catheter body. In some examples, the catheter body may have at least one through-hole, wherein the through-hole is formed by a hole made directly in the outer wall of the catheter body. In some examples, the catheter body may have more than one through-hole. In some examples, the catheter body may have more than one through-hole parallel with at least one other through-hole. In some examples, the catheter body may have more than one through-hole intersecting with at least one other through-hole. In some examples, each through-hole may be a small cluster of small holes. In some examples, each cluster may increase efficiency of charging the first and second balloon for expansion.

In some examples, the catheter body may be a tube, wherein the tube further comprises an outer wall. In some examples, a through-hole may be present in the tube outer wall. In some examples, at least one through-hole may be present in the tube outer wall. In some examples, one through-hole may be present in the tube outer wall. In some examples, two through-holes may be present in the tube outer wall. In some examples, three through-holes may be present in the tube outer wall. In some examples, four through-holes may be present in the tube outer wall. In some examples, five through-holes may be present in the tube outer wall. In some examples, five or more through-holes may be present in the tube outer wall. In some examples, the multiple through-holes may be in sequence toward the distal end of the body. In some examples, the multiple through-holes may be in sequence in a direction away from the distal end of the tube.

In some examples, the at least one through-hole may connect different elements. In some examples, the at least one through-hole may connect the balloon to another element. In some examples, the at least one through-hole may connect the balloon pressurized lumen to another element. In some examples, the at least one through-hole may connect the balloon pressurized lumen to the balloon.

In some examples, the catheter body may have a length of at least about 5 centimeters (“cm”), 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, or any values there between. In some examples, the catheter body may have a length of at most about 110 cm, 105 cm, 100 cm, 95 cm, 90 cm, 85 cm, 80 cm, 75 cm, 70 cm, 65 cm, 60 cm, 55 cm, 50 cm, 45 cm, 40 cm, 35 cm, 30 cm, 25 cm, 20 cm, 15 cm, 10 cm, 5 cm, or any values therebetween. In some examples, the catheter body may have a length from about 40 cm to about 110 cm.

In some examples, the catheter instrument may comprise a balloon. In some examples, the balloon may be from the catheter device. In some examples, the balloon may be from the balloon catheter. In some examples, one balloon may be present. In some examples, more than one balloon may be present. In some examples, two balloons may be present. In some examples, three balloons may be present. In some examples, four balloons may be present. In some examples, five balloons may be present. In some examples, one balloon may be provided through one lumen. In some examples, more than one balloon may be provided through one lumen. In some examples, a first balloon may be in fluid communication a lumen and a second balloon may be in fluid communication with a different lumen. In some examples, a first balloon may be in fluid communication a third lumen and a second balloon may be in fluid communication with a fourth lumen. In some examples, a first balloon may be in fluid communication a third lumen and a first through-hole and a second balloon may be in fluid communication with a fourth lumen and a third through-hole.

In some examples, the balloon may be configured to interact with and apply pressure against a wall. In some examples, the balloon may be configured to interact with the outer wall of the catheter body. In some examples, the balloon may be connected with the outer wall of the catheter body, or tube. In some examples, the balloon may be sealed to the outer wall of the catheter body. In some examples, the balloon may be hermetically sealed to the outer wall of the catheter body. In some examples, the balloon may comprise a balloon material. In some examples, the balloon material may be any acceptable balloon material. In some examples, the balloon material may be any acceptable medical material. In some examples, the balloon material may be selected to allow for diameter expansion at lower pressures. In some examples, the balloon material may be selected to ensure reliable vessel wall support when anchored. In some examples, the balloon material may be a non-metal. In some examples, the balloon material may comprise a plastic or polymer. In some examples, the balloon material may comprise nylon. In some examples, the balloon material may comprise PET (polyethylene terephthalate). In some examples, the balloon material may comprise polyolefin. In some examples, the balloon material may comprise silicone. In some examples, the balloon material may comprise latex. In some examples, the balloon material may comprise polyurethane. In some examples, the balloon material may comprise polyimide ester. In some examples, the balloon material may comprise polyamide polyether block copolymer. In some examples, the balloon material may comprise polyethylene. In some examples, the balloon may comprise a combination of more than one balloon material. In some examples, the balloon may be a compliant wall-like balloon that fits against an outer wall of the catheter body. In some examples, the balloon may be a compliant wall-like balloon that fits against an outer wall of the catheter body in a relaxed state. In some examples, the balloon may be a compliant wall-like balloon that fits against an outer wall of the catheter body in a contracted state.

In some examples, the balloon may have a length of at least about 1 millimeters (“mm”), 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values therebetween. In some examples, the balloon may have a length of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some examples, the balloon may have a length from about 5 mm to about 20 mm. In some embodiments, the balloon may have a length from about 5 mm to about 15 mm. In some examples, the balloon may have a length of about 5 mm. In some embodiments, the balloon may have a length of about 6 mm. In some examples, the balloon may have a length of about 7 mm. In some examples, the balloon may have a length of about 8 mm. In some examples, the balloon may have a length of about 9 mm. In some examples, the balloon may have a length of about 10 mm.

In some examples, the balloon may have an inflated diameter of at least about 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or any values there between. In some examples, the balloon may have an inflated diameter of at most about 40 mm, 39 mm, 38 mm, 37 mm, 36 mm, 35 mm, 34 mm, 33 mm, 32 mm, 31 mm, 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, or any values therebetween. In some examples, the balloon may have an inflated diameter from about 15 mm to about 35 mm. In some examples, the balloon may have an inflated diameter from about 15 mm to about 30 mm. In some examples, the balloon may have an inflated diameter from about 18 mm to about 25 mm.

In some examples, the balloon may be inflated to a pressure of at least about 0.1 atm, 0.2 atm, 0.3 atm, 0.4 atm, 0.5 atm, 0.6 atm, 0.7, 0.8 atm, 0.9 atm, 1 atm, 2 atm, 3 atm, 4 atm, 5 atm, 6 atm, 7 atm, 9 atm, 10 atm, or any values therebetween. In some examples, the balloon may be inflated to a pressure of at most about 10 atm, 9 atm, 8 atm, 7 atm, 6 atm, 5 atm, 4 atm, 3 atm, 2 atm, 1 atm, 0.9 atm, 0.8 atm, 0.7 atm, 0.6 atm, 0.5 atm, 0.4 atm, 0.3 atm, 0.2 atm, 0.1 atm, or any values therebetween. In some examples, the balloon may be inflated to a pressure from about 0.1 atm to about 2 mm. In some examples, the balloon may be inflated to a pressure from about 1 atm to about 2 mm.

In some examples, the catheter instrument is a tube and the tube may be configured to house a lumen. In some examples, the tube may be configured to be a multi-lumen tube. In some examples, one lumen is present. In some examples, two lumens are present. In some examples, three lumens are present. In some examples, four lumens are present. In some examples, five lumens are present. In some examples, more than five lumens are present. In some examples, the lumens may intersect. In some examples, the lumens may not be connected to each other. In some examples, each lumen may be open. In some examples, each lumen may be closed. In some examples, each lumen may be closed from the proximal end. In some examples, each lumen may be closed from the distal end. In some examples, at least one lumen may be open. In some examples, at least one lumen may be closed. In some examples, at least one lumen may be open while all other lumens may be closed. In some examples, at least one lumen may be closed while all other lumens may be open. In some examples, when four lumens may be present, the first lumen may be open while the second, third, and fourth lumens may be closed from the distal ends.

In some examples, the catheter instrument may have a lumen. In some examples, the catheter instrument may have a pass-through lumen. In some examples, the lumen may be an instrument pass-through lumen. In some examples, the lumen may be a balloon catheter pass-through lumen. In some examples, the lumen may be a balloon inflation lumen. In some examples, the lumen may be a pressure monitoring lumen.

In some examples, the catheter instrument, or tube, may have a lumen. In some examples, the lumen may be a guidewire lumen. In some examples, the lumen may be a contrast lumen. In some examples, the lumen may be a pressure medium lumen.

In some examples, the lumen may be a guidewire lumen. In some examples, the lumen may be configured to allow for a guide wire to penetrate. In some examples, the lumen may be configured to allow for a long guide wire to penetrate.

In some examples, the lumen may be a contrast lumen. In some examples, the lumen may be configured to transport lymph fluid. In some examples, the lumen may be configured to transport contrast fluid. In some examples, the contrast lumen may be in fluid communication with a through-hole to allow for contrast transport. In some examples, the contrast lumen may be in fluid communication with the second through-hole for contrast or lymph fluid transport.

In some examples, a lumen may be a pressure medium lumen. In some examples, the lumen may be configured to transport a pressurized medium. In some examples, the pressure medium lumen may extend to the proximal end of the body, or tube. In some examples, the lumen may be configured to transport a pressurized medium through a through-hole. In some examples, the lumen may be configured to transport a pressurized medium through a first through-hole. In some examples, the lumen may be configured to transport a pressurized medium through a first through-hole via the third lumen. In some examples, the lumen may be configured to transport a pressurized medium through a third through-hole. In some examples, the lumen may be configured to transport a pressurized medium through a third through-hole via the fourth lumen.

In some examples, the pressurized medium may be filled into a balloon. In some examples, the balloon may be positioned within the tube. In some examples, the pressurized medium may be used to fill any balloon. In some examples, the pressurized medium may be used to fill a first balloon. In some examples, the pressurized medium may be used to fill a second balloon. In some examples, the pressurized medium may be introduced by an external medium input. In some examples, the balloons, once filled by the pressurized medium, may be configured to block a blood vessel. In some examples, the blood vessel may be the portion containing the lymphatic duct opening. In some examples, filling the balloon in the lymphatic duct opening may block the vessel, creating a negative pressure area. In some examples, the contrast fluid may be input into the negative pressure area via the contrast lumen using an external contrast fluid input. In some examples, a pump may be utilized to extract lymph fluid through a valve structure of the lymphatic duct opening under negative pressure.

In some examples, each lumen may be perforated. In some examples, each lumen may be circularly perforated. In some examples, each lumen may be perforated in a rectangular, or square, shape. In some examples, each lumen may be perforated in a keyhole shape. In some examples, the instrument pass-through lumen may be perforated. In some examples, the balloon catheter pass-through lumen may be perforated. In some examples, the balloon inflation lumen may be perforated. In some examples, the pressure monitoring lumen may be perforated. In some examples, the instrument pass-through lumen, the balloon catheter pass-through lumen, the balloon inflation lumen, and the pressure monitoring lumen are each circularly perforated.

In some examples, each lumen may have a cross section. In some examples, each lumen may have a circular cross-section. In some examples, each lumen may have a rectangular, or square, cross-section. In some examples, the instrument pass-through lumen may have a cross-section. In some examples, the balloon catheter pass-through lumen may have a cross-section. In some examples, the balloon inflation lumen may have a cross-section. In some examples, the pressure monitoring lumen may have a cross-section. In some examples, each lumen cross-section may have a center. In some examples, each lumen may be co-planar with another. In some examples, each lumen may not be in communication with one another.

In some examples, at least one lumen may be disposed along a direction. In some examples, at least one lumen may be disposed in a first direction. In some examples, at least one lumen may be disposed in a second direction. In some examples, at least one lumen may be disposed in a straight-line direction. In some examples, at least one lumen may be disposed in a first straight line direction. In some examples, at least one lumen may be disposed in a second straight line direction. In some examples, at least one lumen may be disposed simultaneously along a direction. In some examples, at least one lumen may be disposed sequentially along a direction. In some examples, at least one lumen may be disposed sequentially along a first straight line direction. In some examples, at least one lumen may be disposed sequentially along a second straight line direction. In some examples, the center of a cross-section of at least one lumen may be disposed sequentially. In some examples, the center of a cross-section of at least one lumen may be disposed sequentially along a first straight line direction. In some examples, the center of a cross-section of at least one lumen may be disposed sequentially along a second straight line direction. In some examples, the center of a cross-section of at least one lumen may be disposed sequentially along a second straight line direction, wherein the second straight line direction intersects the first straight line direction. In some examples, the center of a cross-section of at least one lumen may be disposed sequentially along a second straight line direction, wherein the second straight line direction is orthogonal to the first straight line direction. In some examples, the center of a cross-section of the instrument pass-through lumen, the center of a cross-section of the catheter body, and the center of a cross-section of the balloon catheter pass-through lumen are disposed sequentially along a first straight line direction. In some examples, the center of a cross-section of the balloon inflation lumen and the center of a cross-section of the pressure monitoring lumen are disposed sequentially along a first straight-line direction, and the center of the cross-section of the balloon pressure-filling lumen and the center of the cross-section of the pressure-monitoring lumen are disposed sequentially along a second straight-line direction orthogonal to the first straight-line direction.

In some examples, each lumen may have an aperture R. In some examples, the instrument pass-through lumen may have an aperture R₁ through which the device passes. In some examples, the balloon catheter pass-through lumen may have an aperture R₂ through which the balloon catheter passes. In some examples, the balloon inflation lumen may have an aperture R₃ through which the balloon is inflated. In some examples, the pressure monitoring lumen may have an aperture R₄ through which the pressure is monitored. In some examples, each aperture may be equal in size (R₁=R₂=R₃=R₄). In some examples, each aperture may be different in size (R₁+R₂≠R₃≠R₄). In some examples, at least one aperture may be equal in size to at least one other aperture (R₁=R₂; R₁=R₃; R₁=R₄; R₂=R₃; R₂=R₄; R₃=R₄). In some examples, at least one aperture may be greater than at least one other aperture (R₁>R₂; R₁>R₃; R₁>R₄; R₂>R₃; R₂>R₄; R₃>R₄). In some examples, at least one aperture may be less than at least one other aperture (R₁<R₂; R₁<R₃; R₁<R₄; R₂<R₃; R₂<R₄; R₃<R₄). In some examples, the relationship between the apertures may satisfy the following: R₁>R₂>R₃=R₄.

In some examples, the aperture size may be relative to the cross-section size. In some examples, the aperture may be larger than the cross section. In some examples, the aperture may be smaller than the cross section. In some examples, the aperture may be relatively larger to allow for the instrument to pass through the lumen. In some examples, the aperture size, or diameter, may be designed according to the balloon. In some examples, the aperture size may be designed according to the balloon outer diameter. In some examples, the aperture size may be designed according to the balloon outer diameter in the contracted state.

In some examples, a fifth lumen may be present. In some examples, the fifth lumen may not be connected to any other lumen. In some examples, when a fifth lumen is present, a fourth through-hole may be present. In some examples, when a fifth lumen is present, a fifth through-hole may be present. In some examples, the distal and proximal ends of the fifth lumen may be closed. In some examples, fifth lumen may be connected to the fourth and fifth through-holes. In some examples, the fifth lumen may be configured for blood drain via the through-holes. In some examples, blood may still drain when the first and second balloon block the vessel segment. In some examples, the blood flow while the first and second balloon block the vessel to provide smooth flow, wherein the smooth flow may reduce or avoid surgical risks.

In some examples, the fifth lumen may be open at both ends. In some examples, the fifth lumen may be open at one end. In some examples, the fifth lumen may be closed at both ends. In some examples, the fifth lumen may be blocked at the proximal end by a blocking material. In some examples, the fifth lumen may be blocked at the distal end by a blocking material. In some examples, the fifth lumen may be blocked at both ends, or fully blocked. In some examples, the fully blocked fifth lumen may be manufactured by a conventional extrusion process and the blocking material is inserted.

In some examples, blocking material may be used to close a lumen. In some examples, block material may be used to block any lumen. In some examples, block material may be used to block the second lumen. In some examples, the block material may be used to block the third lumen. In some examples, the block material may be used to block the fourth lumen. In some examples, the block material may be used to block the distal end of any lumen. In some examples, the block material may be used to block the proximal end of any lumen.

In some examples, when five lumens are present within the catheter body, or tube, the five lumens may be arranged. In some examples, the arrangement may provide for the second, third, fourth, and fifth lumens to be positioned around the first lumen. In some examples, the arrangement may be configured to balance the pressure, wherein the arrangement improves delivery efficiency and extends service life. In some examples, the third and fourth lumens may be positioned opposite to one another. In some examples, the first and second lumens may be positioned opposite one another. In some examples, any two lumens may be positioned across from one another. In some examples, when the first lumen is centrally arranged, the first lumen may have a circular cross section. In some examples, when the first lumen is centrally arranged, the second, third, fourth, and fifth lumens may have the same cross-section.

In some examples, the distal and proximal ends of a lumen may be closed. In some examples, the distal and proximal ends of the second lumen may be closed. In some examples, an axial flow pump may be disposed in the second lumen. In some examples, a return pipe may be connected to the second lumen. In some examples, the return pipe may be configured to transport lymph fluid in the second lumen to the venous blood vessel due to the axial flow pump action. In some examples, the axial flow pump may create negative pressure. In some examples, the negative pressure may be configured to pull lymph fluid into the lumen and send the lymph fluid to the venous blood vessel through the return pipe, reducing surgery risk concerns.

In some examples, when the fourth and fifth through-holes are present, the through-holes may be in a sequential order. In some examples, the through-hole order may be fourth, first, second, third, and fifth through-holes in order away from the distal end of the tube.

In some examples, the catheter body may have at least one portion. In some examples, the catheter body may have two or more portions. In some examples, the catheter body may include a main body portion. In some examples, the catheter body may include a tip portion. In some examples, the tip portion may be coupled to an end of the main body portion. In some examples, the tip portion may be coupled to a distal end of the main body portion. In some examples, the instrument may lead to a side of the tip portion through the instrument pass-through lumen. In some examples, the instrument may lead toa distal end of the pressure monitoring lumen. In some examples, the balloon catheter may lead to an end face of the tip portion through the distal end of the lumen.

In some examples, the catheter instrument may comprise an attachment, or coupling, seat. In some examples, the seat may be coupled to a portion of the instrument body. In some examples, the seat may be coupled to the catheter body tube. In some examples, the seat may be coupled to the tip portion. In some examples, the seat may be coupled to the main body portion. In some examples, the seat may be coupled to the proximal end of the catheter body, or tube. In some examples, the seat may be coupled to the distal end of the catheter body. In some examples, the seat may be configured for installation of a hemostatic valve. In some examples, the seat may comprise a port. In some examples, the port may be co-planar with the catheter body. In some examples, the port may be configured for fluid communication with a lumen. In some examples, the port may be in fluid communication with the instrument pass-through lumen. In some examples, the port may be configured for penetration of a diagnostic instrument. In some examples, each lumen may be connected to a seat (i.e., first lumen connected to a first seat, second lumen connected to a second seat, third lumen connected to a third seat, etc.). In some examples, when four lumens and connector seats are present, the connectors provide the following functions: first connector may allow for guide wire insertion; second connector may allow for lymph or contrast fluid transport; third connector and fourth connector may allow for pressurized media transport. In some examples, the connector may be any suitable connector, such as a Luer connector, allowing multiple compatible fluids to be managed using the same pipeline.

In some examples, the seat may comprise a side port. In some examples, the seat may comprise more than one side port. In some examples, the seat may comprise two side ports. In some examples, the seat may comprise three side ports. In some examples, the seat may comprise four side ports. In some examples, each side port may be in communication with a branch, or side/lateral branch. In some examples, a side port may be in fluid communication with a side branch. In some examples, a side port may be in fluid communication with a balloon catheter pass-through lumen. In some examples, a side port may be in fluid communication with a balloon pressure-filling lumen. In some examples, a side port may be in fluid communication with a pressure-monitoring lumen. In some examples, when three side ports are present, each side port may comprise a lumen as disclosed above. In some examples, when three side ports are present, the first side port may be configured for penetration of a balloon catheter. In some examples, when three side ports are present. the second side port may be configured for fluid communication with a side branch for input of a ballon inflation medium. In some examples, when three side ports are present, the third side port may be configured for fluid communication with a side branch for input of a pressure monitoring medium.

In some examples, the catheter instrument may comprise an attachment, or coupling, seat. In some examples, the attachment seat may comprise seat material. In some examples, the seat material may be a metal. In some examples, the seat material may be a non-metal. In some examples, the seat material may be a polymer. In some examples, the seat material may be plastic. In some examples, the seat material may comprise acrylonitrile-butadiene-styrene terpolymer. In some examples, the seat material may comprise polyamide. In some examples, the seat material may comprise polycarbonate. In some examples, the seat material may comprise polyformaldehyde. In some examples, the seat material may comprise a combination thereof. In some examples, the seat material may be configured to provide instrument or therapeutic media. In some examples, the seat material may be selected to be stiff relative to the catheter body. In some examples, the seat material may be configured to facilitate assembly and instrument manipulation.

In some examples, the catheter instrument may further comprise a developing element. In some examples, the catheter instrument may further comprise one or more developing elements. In some examples, the catheter instrument may comprise a first developing element. In some examples, the catheter instrument may comprise a second developing element. In some examples, the catheter instrument may comprise a developing element disposed on the catheter body. In some examples, the catheter instrument may comprise a developing element disposed on the body portion of the catheter body, or tube. In some examples, the developing element may be disposed on an inner wall, outer wall, or interior of the body portion. In some examples, the first developing element may be disposed on an outer wall of the body portion. In some examples, the catheter instrument may comprise a developing element disposed on the tip portion of the catheter body. In some examples, the developing element may be disposed on the interior or side of the tip portion. In some examples, the second developing element may be disposed on a side of the tip portion. In some examples, the developing element may be located in a ballon. In some examples, the developing element may be located in the first balloon. In some examples, the developing element may be located in the second balloon. In some examples, the developing elements may be annular.

In some examples, the developing element may be configured to detection. In some examples, the developing element may be configured for detection before, during, or after surgery. In some examples, the developing element may be configured for detection by an external image detecting device (such as an X-ray detection device). In some examples, the developing element may be configured to allow for accurate positioning by imaging. In some examples, the developing element may be configured to achieve precise positioning for instrument. In some examples, the developing element may be configured to achieve accurate occlusion of the blood vessel segment. In some examples, the developing element may be configured to achieve accurate positioning of the lymphatic vessel orifice.

In some examples, the developing element may comprise a developing element material. In some examples, the developing element material may be a metal, metalloid, or non-metal. In some examples, the developing element material may be gold, platinum, iridium, tantalum, tungsten, or any other acceptable metal or a combination thereof.

In some examples, the catheterization instrument may further comprise a gland. In some examples, the catheter instrument may comprise one or more glands. In some examples, the catheter instrument may comprise a first gland. In some examples, the catheter instrument may comprise a second gland. In some examples, the gland may be disposed at the port. In some examples, the gland may be disposed at a side port. In some examples, the gland may be disposed at a first side port. In some examples, the gland may be disposed at a second side port. In some examples, the catheter instrument may further comprise a hemostatic valve. In some examples, the catheter instrument may comprise a first hemostatic valve. In some examples, the catheter instrument may comprise a second hemostatic valve. In some examples, the hemostatic valve may be secured by a gland. In some examples, the catheter instrument may comprise a first gland disposed at the port, and a first hemostatic valve disposed within the port and secured by the first gland. In some examples, the catheter instrument may comprise a second gland disposed at the first side port, and a second hemostatic valve disposed within the first side port and secured by the second gland. In some examples, the first gland may cooperate with the periphery of the first hemostatic valve. In some examples, the first gland may be screwed down, wherein the first gland presses on the periphery of the first hemostatic valve, causing it to contract to close the hole, thereby providing hemostasis.

In some examples, the valve may be a hemostatic valve. In some examples, the hemostatic valve may be a rotating hemostatic valve. In some examples, the hemostatic valve may be a push-pull hemostatic valve. In some examples, the hemostatic valve may be a push-click hemostatic valve. In some examples, the hemostatic valve may be a Y-connector hemostatic valve. In some examples, the hemostatic valve may be a compression hemostatic valve. In some examples, the hemostatic valve may be a radial compression hemostatic valve. In some examples, the hemostatic valve may be a radial compression hemostatic valve with an axial opening. In some examples, the hemostatic valve may be a cross-cut hemostatic valve. In some examples, the cross-cut hemostatic valve may be in the form of a sheet with a crosscut. In some examples, the cross-cut hemostatic valve may be closed in its natural state. In some examples, the cross-cut hemostatic valve may open upon introduction of the balloon catheter. In some examples, the cross-cut hemostatic valve may be squeezed and wrapped around the surface of the balloon catheter and provide a sealing and hemostatic effect. In some examples, the first hemostatic valve may be a radial compression hemostatic valve. In some examples, the second hemostatic valve may be a cross-cut hemostatic valve. In some examples, the first and second hemostatic valve structure may be any acceptable structure, wherein the shape is not limited.

In some examples, the hemostatic valve may be configured to squeeze and seal due to its own elasticity. In some examples, the hemostatic valve may be configured to prevent blood from flowing out. In some examples, the hemostatic valve may be configured to realize a hemostatic effect. In some examples, the hemostatic valve may comprise valve material. In some examples, the hemostatic material may comprise metal or non-metal. In some examples, the hemostatic material may comprise a polymer. In some examples, the hemostatic material may comprise a plastic. In some examples, the hemostatic material may comprise silicone. In some examples, the hemostatic material may comprise latex. In some examples, the hemostatic material may comprise polyurethane.

In some examples, the catheter instrument may comprise a branch. In some examples, the catheter instrument may comprise a side, or lateral, branch. In some examples, the catheter instrument may comprise one or more side branches. In some examples, the catheter instrument may comprise a first side branch. In some examples, the catheter instrument may comprise a second side branch. In some examples, the catheter instrument may comprise a third side branch. In some examples, the branch may have a proximal end and distal end.

In some examples, the catheter instrument may comprise a fitting. In some examples, the fitting may be a fitting tee (or T-shaped fitting). In some examples, the fitting tee may be a luer fitting tee. In some examples, the luer fitting tee may be provided at an end of the branch. In some examples, the luer fitting tee may be positioned at an end of the side branch. In some examples, the luer fitting tee may be positioned at the distal end of the side branch. In some examples, the luer fitting tee may be positioned at the proximal end of the side branch. In some examples, a luer fitting tee is provided at the proximal end of each of the first side branch and the second side branch. In some examples, the fitting tee may be configured to allow a plurality of mutually compatible fluids to be manage by the same line.

In some examples, the catheter instrument may be a component of a diagnostic device. In some examples, the diagnostic device may comprise more than one catheter. In some examples, the diagnostic device may comprise an examination catheter. In some examples, the diagnostic device may comprise a floating catheter. In some examples, the diagnostic device may comprise an ablation catheter. In some examples, the diagnostic device may comprise a balloon catheter.

In some examples, the catheter instrument may comprise associated diagnostic devices. In some examples, the catheter instrument may comprise balloon catheters. In some examples, the catheter instrument may comprise pumps. In some examples, the catheter instrument may comprise pressure transducers. In some examples, the catheter instrument may comprise a contrast fluid input device. In some examples, the catheter instrument may comprise a lymphatic fluid suction device. In some examples, the catheter instrument may comprise a pressurized medium input device. In some examples, the catheter instrument may comprise an X-ray detection device.

In some examples, the catheter instrument may be a component of a device. In some examples, the catheter instrument may be a component of a heart failure treatment device. In some examples, the catheter instrument may be configured to treat acute congestive heart failure. In some examples, the heat failure treatment device may be used in a venous occlusion regimen, allowing for a favorable prognosis for the treatment of acute congestive heart failure. In some examples, the catheter instrument may be configured to achieve good prognostic results to treat acute congestive heart failure. In some examples, the catheter instrument may be configured for treatment of heart failure by enhancing the absorption and drainage functions of the lymphatic system. In some examples, the enhancement may more efficiently transfer the stagnant interstitial fluid retention and improve heart failure treatment.

In some examples, the catheter may be configured to transport contrast fluid. In some examples, the catheter may be configured to transport lymph fluid. In some examples, the catheter may be configured to transport contrast fluid to the lymphatic vessel. In some examples, the catheter may be configured to transport lymphatic fluid from the lymphatic vessel. In some examples, lymph fluid may flow out of the catheter device from the second connector and can then enter the human body's venous vessels again through other catheters via extracorporeal circulation, thereby returning to the human body's circulation.

In some examples, the tube, or catheter body, may be designed for entry into the blood vessel. In some examples, the tube may be designed for smooth entry into the blood vessel. In some examples, smooth entry may be designed for by tapering an end of the multi-lumen tube. In some examples, the tapering may be at a distal end of the multi-lumen tube.

### Balloon Sheath

In some examples, a balloon sheath may be disclosed. In some examples, the balloon sheath may be applied to a heart failure treatment device. In some examples, the balloon sheath may comprise a sheath. In some examples, the sheath may be a main, or primary, sheath. In some examples, the sheath may be a secondary sheath. In some examples, the balloon sheath may comprise a balloon. In some examples, the balloon sheath may comprise a connection seat. In some examples, the connection seat may be a main connection seat, or a first connection seat. In some examples, the connection seat may be a secondary connection seat. In some examples, the balloon sheath may comprise a branch.

In some examples, the balloon sheath may comprise a main sheath. In some examples, the main sheath may comprise a lumen. In some examples, the main sheath may comprise more than one lumen. In some examples, the main sheath may comprise a main, or first, lumen. In some examples, the main sheath may comprise a secondary lumen. In some examples, the main sheath may comprise a pressure-filled lumen. In some examples, the main sheath may comprise a pressure-filled lumen, wherein the pressure-filled lumen may be configured to supply a pressure-filled medium. In some examples, the main sheath may comprise a first pressure-filled lumen. In some examples, the main sheath may comprise a second pressure-filled lumen. In some examples, the more than one of the lumens may not be in fluid communication.

In some examples, the main sheath may comprise a tip portion, or first tip portion. In some examples, the tip portion may be adjacent to an end of the main sheath. In some examples, the tip portion may be adjacent to the distal or proximal end of the main sheath. In some examples, the tip portion may be exposed to the main lumen due to a perforation at the distal or proximal end. In some examples, the tip portion end face may be exposed to the main lumen due to a perforation of the distal end. In some examples, the tip portion may be exposed to the secondary lumen due to a perforation at the distal or proximal end. In some examples, the tip portion side face may be exposed to the secondary lumen due to a perforation of the distal end. In some examples, the diagnostic instrument may be threaded through the first tip portion of the main sheath. In some examples, the diagnostic instrument may be threaded through the first tip portion end face of the main sheath. In some examples, the balloon catheter may be threaded through the first tip portion of the main sheath. In some examples, the balloon catheter may be threaded through the first tip portion side face of the main sheath. In some examples, the configuration of the tip portion may reduce resistance with the blood vessel. In some examples, the configuration of the tip portion may reduce resistance with the blood vessel when traveling, allowing for smoother access.

In some examples, the tip portion may comprise an instrument material. In some examples, the tip portion may comprise an instrument material. In some examples, the instrument material may comprise a metal. In some examples, the instrument material may comprise a non-metal. In some examples, the instrument material may comprise a polymer. In some examples, the instrument material may comprise plastic. In some examples, the instrument material may comprise polyamide. In some examples, the instrument material may comprise polyether block polyamide. In some examples, the instrument material may comprise acrylonitrile-butadiene-styrene terpolymer. In some examples, the instrument material may comprise polyethylene glycol p-toluene dicarboxylate. In some examples, the instrument material may be polyurethane. In some examples, the instrument material may be polyimide ester. In some examples, the instrument material may be silicone. In some examples, the instrument material may be selected to reduce resistance with the blood vessel for smoother entry.

In some examples, the main sheath may comprise a main lumen. In some examples, the main lumen may be configured to guide a diagnostic instrument. In some examples, the main lumen may be configured to guide a therapeutic instrument. In some examples, the main sheath may comprise a secondary lumen. In some examples, the secondary lumen may be configured to guide the balloon catheter.

In some examples, the balloon sheath may comprise a lumen. In some examples, the balloon sheath may comprise more than one lumen. In some examples, the more than one lumen may not be in fluid communication. In some examples, the more than one lumen may be in fluid communication. In some examples, the more than one lumen may be orthogonal. In some examples, the more than one lumen may be coplanar.

In some examples, the balloon sheath may comprise a first balloon. In some examples, the first balloon may be configured to interact with and apply pressure against a wall. In some examples, the balloon may be configured to interact with the outer wall of the main sheath. In some examples, the balloon may be connected with the outer wall of the main lumen. In some examples, the first balloon may be connected to a lumen. In some examples, the first balloon may be connected to the first pressure-filled lumen. In some examples, the first balloon may be connected to the second pressure-filled lumen.

In some examples, each lumen may have a cross-sectional shape. In some examples, the cross-sectional shape may be any suitable shape. In some examples, the cross-sectional shape may be circular, semi-circular, rectangular, or any other shape. In some examples, the cross-sectional shape of the main lumen and the secondary lumen may be circular.

In some examples, each lumen may have an aperture. In some examples, the main lumen and the secondary lumen may each have an aperture. In some examples, the main lumen aperture (R₁) may have a size relative to the secondary lumen aperture (R₂) size. In some examples, R₁=R₂. In some examples, R₁>R₂. In some examples, R₁<R₂. In some examples, R₁≠R₂. In some examples, R₁ may be configured to enable smooth passage of a diagnostic or therapeutic instrument. In some examples, R₂ may be configured to enable passage of a balloon. In some examples, R₂ may be configured to enable passage of the second balloon. In some examples, R₂ may be configured to enable passage of the second balloon in a contracted state.

In some examples, the lumens may have an arrangement. In some examples, the main and secondary lumen may be positioned together. In some examples, the first pressure-filled lumen and the second pressure-filled lumen may be located on the side of the main and secondary lumen. In some examples, the first pressure-filled lumen and the second pressure-filled lumen may be located on both sides of the main and secondary lumen. In some examples, the main sheath may be molded using an extrusion process. In some examples, the main lumen and secondary lumen may be positioned to allow for spaces on either side, wherein the first and second pressure-filled lumens are that space.

In some examples, the main sheath may comprise a through-hole. In some examples, the main sheath may comprise at least one through-hole. In some examples, the main sheath may comprise one through-hole, two through-holes, three through-hole, four through-holes, five through-holes, or more than five through-holes. In some examples, the through-holes may connect the first pressure-filled lumen and the second pressure-filled lumen to the first balloon.

In some examples, the first balloon may be configured to interact with and apply pressure against a wall. In some examples, the first balloon may be configured to interact with the outer wall of the main sheath. In some examples, the first balloon may be connected with the outer wall of the main sheath. In some examples, the first balloon may be sealed to the outer wall of the main sheath. In some examples, the first balloon may be hermetically sealed to the outer wall of the main sheath. In some examples, the first balloon may comprise a balloon material. In some examples, the balloon material may be any acceptable balloon material. In some examples, the balloon material may be any acceptable medical material. In some examples, the balloon material may be selected to allow for diameter expansion at lower pressures. In some examples, the balloon material may be selected to ensure reliable vessel wall support when anchored. In some examples, the balloon material may be a non-metal. In some examples, the balloon material may comprise a plastic or polymer. In some examples, the balloon material may comprise nylon. In some examples, the balloon material may comprise PET (polyethylene terephthalate). In some examples, the balloon material may comprise polyolefin. In some examples, the balloon material may comprise silicone. In some examples, the balloon material may comprise latex. In some examples, the balloon material may comprise polyurethane. In some examples, the balloon material may comprise polyimide ester. In some examples, the balloon material may comprise polyamide polyether block copolymer. In some examples, the balloon material may comprise polyethylene. In some examples, the balloon may comprise a combination of more than one balloon material. In some examples, the balloon may be a compliant wall-like balloon that fits against an outer wall of the main sheath. In some examples, the balloon may be a compliant wall-like balloon that fits against an outer wall of the main sheath in a relaxed state. In some examples, the balloon may be a compliant wall-like balloon that fits against an outer wall of the main sheath in a contracted state.

In some examples, the first balloon may be configured to efficiently anchor after expansion to provide a reliable support in the blood vessel. In some examples, the first balloon may be configured to adopt a tubular wall-like design. In some examples, the first balloon maybe configured to minimize the resistance to travel in the blood vessel. In some examples, the first balloon may be configured to reduce the harm to the human body.

In some examples, the balloon sheath may comprise a balloon catheter. In some examples, the balloon catheter may be a tube. In some examples, the balloon catheter may comprise a tube. In some examples, the tube may be a multi-lumen tube. In some examples, the tube may be a single-lumen tube. In some examples, the balloon catheter may comprise both a single lumen tube and a multi-lumen tube. In some examples, the balloon catheter may comprise a balloon. In some examples, the balloon catheter may comprise a balloon (second balloon). In some examples, the balloon catheter may comprise a connection seat.

In some examples, the balloon catheter may comprise a multi lumen tube. In some examples, the multi lumen tube may comprise two lumens, three lumens, four lumens, five lumens, or six or more lumens. In some examples, the multi lumen tube may comprise a guidewire feedthrough lumen. In some examples, the multi lumen tube may comprise a pressure-filled media delivery lumen. In some examples, the multi lumen tube may comprise a contrast agent delivery lumen. In some examples, each lumen of the multi lumen tube may not be in fluid communication with each other. In some examples, each lumen of the multi lumen tube may be in fluid communication with each other. In some examples, the contrast agent delivery lumen may be exposed on the outer wall of the multi-lumen tube.

In some examples, the guidewire entry lumen may be perforated. In some examples, the guidewire entry lumen may be circularly perforated and its center axis coincides with the center axis of the multi-lumen tube. In some examples, the pressure-filled medium delivery lumen may have a curved perforations surrounding the guidewire entry lumen. In some examples, the contrast agent delivery lumen may have a curved perforations surrounding the guidewire entry lumen. In some examples, the multi lumen tube may comprise a material and that material may include at least one of a polyamide, a polyether-blocked polyamide.

In some examples, the balloon catheter may comprise a single lumen tube. In some examples, the single lumen tube may be connected to the distal end of the guidewire penetration lumen. In some examples, the second balloon may be sealingly connected to the outer wall of the multi lumen tube. In some examples, the second balloon may be sealingly connected to the outer wall of the single lumen tube. In some examples, the second balloon may be connected to the distal end of the pressurized media delivery lumen. In some examples, the coupling seat may be connected to the proximal end of the multi-lumen tube. In some examples, the coupling seat may comprise a first connector. In some examples, the coupling seat may comprise a first connector communicating with the guidewire threading lumen. In some examples, the coupling seat may comprise a second connector. In some examples, the coupling seat may comprise a second connector, communicating with the pressure-filled medium delivery lumen. In some examples, the coupling seat may comprise a third connector. In some examples, the coupling seat may comprise a third connector communicating with the contrast agent delivery lumen. In some examples, the first connector may be configured for threading of the guidewire. In some examples, the second connector may be configured for inputting the pressure-filled medium. In some examples, the third connector may be configured for inputting the contrast agent.

In some examples, the single lumen tube may include a second tip portion. In some examples, the second tip portion may be adjacent to its distal end. In some examples, the second tip portion may reduce the resistance of the balloon catheter as it travels through the blood vessel, allowing for smoother entry. In some examples, single lumen may be made of a softer material than the tip portion, and may for example comprise at least one of polyurethane, polyurethane, polyimide, polyether block polyamide, and silicone.

In some examples, the single lumen tube may further comprise a developing element. In some examples, the outer wall of the single lumen tube may be provided with a developing element. In some examples, the developing element may be designed within the second balloon. In some examples, the developing element may be detected during surgery using an external image detection device (e.g., an X-ray detection device) so that its position can be accurately known and precise positioning can be realized. In some examples, the developing element may comprise a developing element material. In some examples, the developing element material may be a metal, metalloid, or non-metal. In some examples, the developing element material may be gold, platinum, iridium, tantalum, tungsten, or any other acceptable metal or a combination thereof.

In some examples, the balloon sheath may comprise a branch. In some examples, the balloon sheath may comprise more than one branch. In some examples, the balloon sheath may comprise one branch, two branches, three branches, four branches, five branches, or more than five branches. In some examples, each balloon sheath may be a lateral branch.

In some examples, the balloon catheter may comprise a second balloon. In some examples, the second balloon may be a non-compliant balloon. In some examples, the second balloon may comprise material of which may include at least one of polyamide, polyether block polyamide, and polyethylene. In some examples, the diameter of the second balloon, after expanding to a certain value, may remain at that value regardless of changes in external pressure. In some examples, the diameter retention may allow for precise sealing of the blood vessel at the specified location without damaging the blood vessel due to over-expansion.

In some examples, the balloon catheter may further comprise a reinforcing sleeve. In some examples, the reinforcing sleeve may be provided at the connection between the multi-lumen tube and the connection seat. In some examples, the reinforcing sleeve may seal the multi-lumen tube to the connection seat. In some examples, the reinforcing sleeve may be used to seal the connection between the multi-lumen tube and the connection seat firmly. In some examples, the reinforcing sleeve may prevent excessive bending at the connection. In some examples, the reinforcing sleeve may comprise a material which may include at least one of a polyamide, an acrylonitrile-butadiene-styrene terpolymer, a polyolefin, a polyether block polyamide, and the like, and may also include a metal oxide such as barium sulfate.

In some examples, the balloon sheath may comprise a side, or lateral, branch. In some examples, the lateral branch may be in fluid communication with a side port. In some examples, the balloon sheath may comprise a first lateral branch. In some examples, the first lateral branch may be in fluid communication with a first side port. In some examples, the first side branch may be configured to fill the main lumen with a flushing fluid. In some examples, the balloon sheath may comprise a second lateral branch. In some examples, the second lateral branch may be in fluid communication with a second side port. In some examples, the second lateral branch may be configured to input a pressure-filled medium. In some examples, the second lateral branch may be configured to input a pressure-filled medium from the first pressure-filled lumen. In some examples, the second lateral branch may be configured to input a pressure-filled medium from the second pressure-filled lumen. In some examples, the balloon sheath may comprise a third lateral branch. In some examples, the third lateral branch may be in fluid communication with a third side port. In some examples, the third lateral branch may be configured to supply a pressurized medium to the secondary lumen. In some examples, the supply of a pressurized medium to the secondary lumen may allow the secondary lumen to fill with a flushing fluid. In some examples, the supply of a pressurized medium to the secondary lumen may allow for blood draw.

In some examples, each branch may be a single tube. In some examples, each branch may be a single lumen tube. In some examples, each branch may be an array of lumens. In some examples, each branch may be flexible, or bendable. In some examples, each branch may be capable of withstanding pressure. In some examples, each branch may be capable of withstanding a certain amount of pressure. In some examples, each branch may provide a therapeutic medium pathway. In some examples, each branch may may provide a flushing medium pathway. In some examples, each branch may be configured for body fluid collection.

In some examples, each branch may comprise a branch material. In some examples, the branch may comprise one or more branch materials. In some examples, the branch material may be metal. In some examples, the branch material may be non-metal. In some examples, the branch material may be polymer. In some examples, the branch material may be plastic. In some examples, the branch material may be polyethylene. In some examples, the branch material may be polyvinyl chloride. In some examples, the branch material may be polyether block polyamide.

In some examples, each branch may be connected to a fitting tee. In some examples, the fitting tee may be connected to the proximal end of the fitting tee. In some examples, the fitting tee may be connected to the distal end of the fitting tee. In some examples, the fitting tee may be luer fitting tee. In some examples, a fitting tee may be connected to a first side, or lateral, branch. In some examples, a fitting tee may be connected to a second side, or lateral, branch. In some examples, a fitting tee may be connected to a third side, or lateral, branch. In some examples, the fitting tee may be configured to allow for a plurality of compatible fluids to be administered along the same line.

In some examples, the balloon sheath may comprise a connector, or connector seat. In some examples, the connection seat may be a main connection seat, or a first connection seat. In some examples, the main connector may be connected to the main sheath. In some examples, the main connector may be connected to the proximal end or the distal end of the main sheath. In some examples, the main connector may further comprise a port. In some examples, the main connector may further comprise a first port. In some examples, the first port may be in fluid communication with a lumen. In some examples, the first port may be in fluid communication with a main lumen. In some examples, the main connector may further comprise a second port. In some examples, the second port may be in fluid communication with a lumen. In some examples, the second port may be in fluid communication with a secondary lumen. In some examples, the main connector may further comprise a side port. In some examples, the main connector may further comprise a first side port. In some examples, the first side port may be in fluid communication with a lumen. In some examples, the first side port may be in fluid communication with a main lumen. In some examples, the main connector may further comprise second side ports. In some examples, the second side ports may be in fluid communication with a lumen. In some examples, the second side ports may be in fluid communication with a first pressure-filled, or pressurized, lumen. In some examples, the second side ports may be in fluid communication with a second pressure-filled, or pressurized, lumen.

In some examples, the balloon sheath may comprise a connector, or connector seat. In some examples, the connection seat may comprise a first port. In some examples, the first port may be configured to thread a diagnostic instrument. In some examples, the first port may be configured to thread a therapeutic instrument. In some examples, the first port may be configured to thread a diagnostic, or therapeutic, instrument into the main lumen. In some examples, the connection seat may comprise a second port. In some examples, the second port may be configured for communication with the secondary sheath tube.

In some examples, the connection seat may be a secondary connection seat. In some examples, the secondary connector may be connected to the secondary sheath. In some examples, the secondary connector may be connected to the proximal end or the distal end of the secondary sheath. In some examples, the secondary connector may comprise a port. In some examples, the secondary connector may comprise a third port. In some examples, the third port may be configured for threading a balloon catheter. In some examples, the third port may be configured to thread a balloon catheter into the secondary lumen. In some examples, the secondary connector may comprise a side port. In some examples, the secondary connector may comprise a third side port. In some examples, the third port may be in fluid communication with a secondary sheath.

In some examples, the connection seat may comprise seat material. In some examples, the seat material may comprise metal. In some examples, the seat material may comprise non-metal. In some examples, the seat material may comprise polymer. In some examples, the seat material may comprise plastic. In some examples, the seat material may comprise acrylonitrile-butadiene-styrene terpolymer. In some examples, the seat material may comprise a polyamide. In some examples, the seat material may comprise a polycarbonate. In some examples, the seat material may comprise a polyformaldehyde. In some examples, the seat material may be configured to be stiff relative to the primary sheath. In some examples, the seat material may be configured to be stiff relative to the secondary sheath. In some examples, the seat material may be configured to facilitate assembly and instrument manipulation.

In some examples, the connection seat may be configured to provide access. In some examples, the connection seat may be configured to provide access to other instruments. In some examples, the connection seat may be configured to provide access to treatment media. In some examples, the connection seat may be configured for hemostatic valve installation.

In some examples, the balloon sheath may further comprise a valve. In some examples, the balloon sheath may comprise one or more valves. In some examples, the balloon sheath may comprise one sheath, two sheaths, three sheaths, four sheaths, five sheaths, or six or more sheaths. In some examples, the valve may be a hemostatic valve. In some examples, the balloon sheath may be a primary, or main, hemostatic valve. In some examples, the primary hemostatic valve may be disposed within the primary connection seat. In some examples, the balloon sheath may be a secondary, or vice, hemostatic valve. In some examples, the secondary hemostatic valve may be disposed within the secondary connection seat. In some examples, each hemostatic valve may have elasticity. In some examples, each hemostatic valve may be configured to squeeze and seal, wherein the seal may prevent blood outflow. In some examples, each hemostatic valve may be configured to squeeze and seal, realizing a hemostatic effect.

In some examples, the valves may comprise valve material. In some examples, the valve material may comprise metal. In some examples, the valve material may comprise non-metal. In some examples, the valve material may comprise polymer. In some examples, the valve material may comprise plastic. In some examples, the valve material may comprise silicone. In some examples, the valve material may comprise latex. In some examples, the valve material may comprise polyurethane. In some examples, the valve material may comprise polyethylene. In some examples, the valve may comprise a combination of one or more valve materials.

In some examples, the balloon sheath may further comprise a dilator. In some examples, the balloon sheath may further comprise a primary, or main, dilator. In some examples, the balloon sheath may further comprise a secondary dilator. In some examples, the dilators may be intermediate auxiliary instruments for balloon sheath placement. In some examples, the dilators may be intermediate auxiliary instruments for balloon catheter placement. In some examples, the main dilator may be a single lumen catheter. In some examples, the main dilator may comprise a dilator seat. In some examples, the main dilator may provide access for the guidewire to enter the main sheath. In some examples, the main dilator single lumen catheter may comprise a tapering segment at the head end, wherein the taper facilitates the assembly of the main dilator with the main sheath through the hemostatic valve. In some examples, the main dilator single lumen catheter may comprise at least one of polyethylene, polyvinyl chloride, and polyether block polyamide. In some examples, the main dilator single lumen catheter may incorporate a metal oxide such as barium sulfate, wherein the metal oxide allows the dilator catheter to be developable. In some examples, the dilator seat may comprise a luer fitting, which may be of a material consistent with the material of the single lumen catheter.

In some examples, the balloon sheath may be configured to provide access to a subject. In some examples, the balloon sheath may be configured to provide diagnostic instrument, or device, access. In some examples, the balloon sheath may be configured to provide therapeutic instrument, or device, access. In some examples, the balloon sheath may be configured to provide balloon catheter access. In some examples, the balloon sheath may be configured to seal a blood vessel of a subject. In some examples, the balloon sheath may be configured to seal a blood vessel of a subject using a balloon.

In some examples, the balloon sheath may be configured to place a balloon catheter after sheath placement in the human body. In some examples, the balloon sheath may be configured to place a diagnostic instrument after sheath placement in the human body. In some examples, the balloon sheath may be configured to guide placement of a diagnostic instrument or balloon catheter. In some examples, a guide member, such as a short or long guidewire, may be configured to guide placement of a diagnostic instrument or balloon catheter. In some examples, a vessel may be occluded using a first balloon. In some examples, an associated diagnostic or therapeutic operation may be completed after introduction of a second balloon from a balloon catheter.

In some examples, a first balloon from the balloon sheath and second balloon from the balloon catheter may be passed through the balloon sheath. In some examples, a first balloon from the balloon sheath and second balloon from the balloon catheter may be passed to a subject. In some examples, a first balloon from the balloon sheath and second balloon from the balloon catheter may be configured to block a vein. In some examples, a first balloon from the balloon sheath and second balloon from the balloon catheter may be configured to intermittently block a vein. In some examples, a first balloon from the balloon sheath and second balloon from the balloon catheter may be configured to intermittently block the inferior vena cava and the subclavian vein. In some examples, the intermittent blocking may be configured to reduce return of venous blood to the heart. In some examples, the intermittent blocking may be configured to lower the preload of the heart. In some examples, the intermittent blocking may be configured to lower ventricular wall stress. In some examples, a first balloon from the balloon sheath and second balloon from the balloon catheter may be configured to synchronize. In some examples, a first balloon from the balloon sheath and second balloon from the balloon catheter may be configured to inflate. In some examples, a first balloon from the balloon sheath and second balloon from the balloon catheter may be configured to deflate. In some examples, a first balloon from the balloon sheath and second balloon from the balloon catheter may be configured to both inflate and deflate. In some examples, a first balloon from the balloon sheath and second balloon from the balloon catheter may be configured to both inflate and deflate using an external pressure-filling medium input device. In some examples, a first balloon from the balloon sheath and second balloon from the balloon catheter may be configured to inflate and deflate in accordance with cardiac rhythm. In some examples, a first balloon from the balloon sheath and second balloon from the balloon catheter may be configured to inflate and deflate in accordance with cardiac rhythm, generating a dual hemodynamic effect. In some examples, a first balloon from the balloon sheath and second balloon from the balloon catheter may be configured to inflate and deflate in accordance with cardiac rhythm, elevating diastolic blood pressure. In some examples, a first balloon from the balloon sheath and second balloon from the balloon catheter may be configured to inflate and deflate in accordance with cardiac rhythm, elevating coronary perfusion. In some examples, a first balloon from the balloon sheath and second balloon from the balloon catheter may be configured to inflate and deflate in accordance with cardiac rhythm, lowering the afterload of the heart. In some examples, a first balloon from the balloon sheath and second balloon from the balloon catheter may be configured to inflate and deflate in accordance with cardiac rhythm, improving the ejection of the left ventricle.

In some examples, an external contrast fluid input device may be utilized to allow contrast fluid to be injected into the blood vessel though the contrast delivery lumen. In some examples, the contrast delivery lumen may be designed on a side of the second balloon away from the single lumen tube.

In some examples, the main lumen of the balloon sheath may be configured to be passed into a diagnostic instrument, or device. In some examples, the main lumen of the balloon sheath may be configured to be passed into a diagnostic instrument for relevant testing. In some examples, the main lumen of the balloon sheath may be configured to be passed into a diagnostic instrument for relevant treatment. In some examples, the main lumen of the balloon sheath may be configured to be passed into a diagnostic instrument before, during, or after treatment. In some examples, the diagnostic device may be a floating catheter, an ablation catheter, a balloon catheter, or any suitable catheter. In some examples, the diagnostic device may be a Swan-Ganz catheter. In some examples, the diagnostic device may be a coronary balloon, a coronary stent, or any suitable device.

In some examples, the balloon sheath may be configured for treatment. In some examples, the balloon sheath may be configured for treatment of heart failure. In some examples, the balloon sheath may be configured for treatment of acute congestive heart failure. In some examples, the balloon sheath may be configured for treatment of acute congestive heart failure with a good prognosis.

In some examples, the balloon sheath may be a component of a heart failure device. In some examples, the heart failure treatment device may include an associated diagnostic device. In some examples, the heart failure treatment device may include a balloon catheter. In some examples, the heart failure treatment device may include an external device. In some examples, the heart failure treatment device may include a pump. In some examples, the heart failure treatment device may be configured for treatment protocols of venous occlusion. In some examples, the heart failure treatment device may be configured for treatment protocols of intra-aortic counter pulsation. In some examples, the heart failure treatment device may be configured for treatment protocols of intra-aortic counter pulsation, which can lead to good prognostic results in the treatment of acute congestive heart failure.

In some examples, the heart failure treatment device may comprise a balloon catheter. In some examples, the balloon catheter may be nested with the primary sheath. In some examples, the balloon catheter may be assembled with the primary sheath. In some examples, the balloon catheter may be nested with the secondary sheath. In some examples, the balloon catheter may be assembled with the secondary sheath. In some examples, the balloon catheter may be sized and structured for need.

The balloon catheter system may further include a tearable or detachable sheath puncture kit as well as consumable accessories. In some examples, a detachable sheath for inserting the balloon catheter system may be present. In some examples, the detachable sheath may be configured to be inserted into a puncture site at the femoral vein of a subject. In some examples, the detachable sheath may comprise a first detachable arm and a second detachable arm that allow the detachable sheath to be torn and removed after placement of the first balloon at the first target location.

In some examples, the detachable sheath may further comprise a tapered dilator for enlarging the insertion site at the femoral vein. In some examples, the tapered dilator may have a shaft length of at least about 1 cm, 2 cm, 3 cm, 4 cm, 5 cm, 6 cm, 7 cm, 8 cm, 9 cm, 10 cm, 12 cm, 14 cm, 16 cm, 18 cm, 20 cm, 22 cm, 24 cm, 26 cm, 28 cm, 30 cm, 35 cm, 40 cm, 50 cm, or any values there between. In some examples, the tapered dilator may have a shaft length of at most about 50 cm, 40 cm, 35 cm, 30 cm, 28 cm, 26 cm, 24 cm, 22 cm, 20 cm, 18 cm, 16 cm, 14 cm, 12 cm, 10 cm, 9 cm, 8 cm, 7 cm, 6 cm, 5 cm, 4 cm, 3 cm, 2 cm, 1 cm, or any values therebetween. In some examples, the tapered dilator may have a shaft length from about 10 cm to about 50 cm. In some examples, the tapered dilator may have a shaft length from about 20 cm to about 40 cm. In some examples, the tapered dilator may have a shaft length of about 35 cm.

### Nested Balloon Catheters

The disclosed embodiments provide a catheter device and a heart failure treatment apparatus to improve the effect of heart failure treatment.

In some examples, the catheter device may comprise a balloon catheter. In some examples, the catheter device may comprise more than one balloon catheter. In some examples, the catheter device may comprise one ballon catheter, two balloon catheters, three balloon catheters, four balloon catheters, or five or more balloon catheters. In some examples, the catheter device may comprise more than one balloon catheter. In some examples, the more than one balloon catheters may be situated in relation to one another. In some examples, the balloon catheter may be an inner balloon catheter. In some examples, the balloon catheter may be an outer balloon catheter. In some examples, the inner balloon catheter and outer balloon catheter may be nested. In some examples, the inner balloon catheter and outer balloon catheter may be parallel. In some examples, the inner balloon catheter and outer balloon catheter may be co-planar. In some examples, the inner balloon catheter and outer balloon catheter may be orthogonal. In some examples, the inner balloon catheter and outer balloon catheter may be assembled.

In some examples, the catheter device may comprise an inner ballon catheter. In some examples, the inner ballon catheter may comprise a first multi-lumen tube. In some examples, the inner ballon catheter may comprise a first single-lumen tube. In some examples, the inner ballon catheter may comprise a first balloon. In some examples, the inner ballon catheter may comprise a first hub.

In some examples, the inner balloon catheter may comprise a first multi-lumen tube. In some examples, the first multi-lumen tube may comprise a more than one lumen. In some examples, the first multi-lumen tube may comprise two lumens, three lumens, four lumens, five lumens, or six or more lumens. In some examples, the first multi-lumen tube may comprise a first lumen. In some examples, the first multi-lumen tube may comprise a second lumen. In some examples, the first multi-lumen tube may comprise a third lumen. In some examples, the first multi-lumen tube may comprise a fourth lumen. In some examples, the lumens of the multi-lumen tube may be orthogonal. In some examples, the lumens of the multi-lumen tube may be parallel. In some examples, the lumens of the multi-lumen tube may be co-planar. In some examples, the lumens of the multi-lumen tube may be not in fluid connection. In some examples, the lumens of the multi-lumen tube may be in fluid connection.

In some examples, the inner ballon catheter may comprise a first single-lumen tube. In some examples, the first single-lumen tube may comprise a first lumen. In some examples, the first single-lumen tube may be connected to a first lumen. In some examples, the first single-lumen tube may be connected to the distal end of the first lumen. In some examples, the first single-lumen tube may be connected to the proximal end of the first lumen.

In some examples, the first single-lumen tube may comprise a body portion. In some examples, the first single-lumen tube may comprise a tip portion. In some examples, the tip portion may be connected to the body portion. In some examples, the tip portion may be connected to the end of the body portion. In some examples, the tip portion may be connected to the distal end of the body portion. In some examples, the tip portion may be connected to the proximal end of the body portion.

In some examples, the tip portion may be welded to the body portion. In some examples, the tip portion may be adhered to the body portion. In some examples, the tip portion may comprise metal, non-metal, plastic, or polymer. In some examples, the tip portion may include polyamide. In some examples, the tip portion may include polyurethane. In some examples, the tip portion may include polyether block polyamide. In some examples, the tip portion may include silicone. In some examples, the body portion may comprise metal, non-metal, plastic, or polymer. In some examples, the body portion may include polyamide. In some examples, the body portion may include polyurethane. In some examples, the body portion may include polyether block polyamide. In some examples, the body portion may include silicone. In some examples, the body portion may be of harder material than the tip portion. In some examples, the tip portion may be of softer material than the body portion. In some examples, the tip portion material may be configured to reduce the resistance of the catheter device. In some examples, the tip portion material may be configured to reduce the resistance of the catheter device in the blood vessel, to enable smoother entry. In some examples, the tip portion shape may be configured to reduce the resistance of the catheter device. In some examples, the tip portion shape may be configured to reduce the resistance of the catheter device in the blood vessel, to enable smoother entry.

In some examples, the inner ballon catheter may comprise a first balloon. In some examples, the first balloon may be connected to the first multi-lumen tube. In some examples, the first balloon may be connected to the outer wall of the first multi-lumen tube. In some examples, the first balloon may be connected to the outer wall of the first multi-lumen tube, wherein a seal is formed. In some examples, the first balloon may be connected to the first single-lumen tube. In some examples, the first balloon may be connected to the outer wall of the first single-lumen tube. In some examples, the first balloon may be connected to the outer wall of the first single-lumen tube, wherein a seal is formed. In some examples, the first balloon may be connected to a second lumen. In some examples, the first balloon may be connected to an end of the second lumen. In some examples, the first balloon may be connected to the distal end of the second lumen. In some examples, the first balloon may be connected to the proximal end of the second lumen.

In some examples, the first balloon may be a single layer balloon. In some examples, the first balloon may be a multi-layered balloon. In some examples, the first balloon may be a double layer balloon. In some examples, the first balloon may be a triple layer balloon. In some examples, the double layer first balloon may have an outer layer material. In some examples, the double layer first balloon may have an inner layer material. In some examples, the inner layer material and the outer layer material may be connected. In some examples, the inner layer material and the outer layer material may be attached to one another. In some examples, the inner layer material and the outer layer material may have a relative hardness. In some examples, the relative hardness of the inner layer material may be equal to the relative hardness of the outer layer material. In some examples, the relative hardness of the inner layer material may be less than the relative hardness of the outer layer material. In some examples, the relative hardness of the inner layer material may be greater than the relative hardness of the outer layer material.

In some examples, the inner layer material may comprise a metal. In some examples, the inner layer material may comprise a non-metal. In some examples, the inner layer material may comprise a polymer. In some examples, the inner layer material may comprise a plastic. In some examples, the inner layer material may comprise polyamide. In some examples, the inner layer material may comprise polyether block polyamide. In some examples, the inner layer material may comprise polyethylene terephthalate. In some examples, the inner layer material may comprise polyurethane. In some examples, the inner layer material may comprise thermoplastic elastomer. In some examples, the inner layer material may comprise silicone. In some examples, the inner layer material may comprise latex. In some examples, the inner layer material may comprise at least one of polyamide, polyether block polyamide, or polyethylene terephthalate.

In some examples, the outer layer material may comprise a metal. In some examples, the outer layer material may comprise a non-metal. In some examples, the outer layer material may comprise a polymer. In some examples, the outer layer material may comprise a plastic. In some examples, the outer layer material may comprise polyamide. In some examples, the outer layer material may comprise polyether block polyamide. In some examples, the outer layer material may comprise polyethylene terephthalate. In some examples, the outer layer material may comprise polyurethane. In some examples, the outer layer material may comprise thermoplastic elastomer. In some examples, the outer layer material may comprise silicone. In some examples, the outer layer material may comprise latex. In some examples, the outer layer material may comprise at least one of polyether block polyamide, polyurethane, thermoplastic elastomer, silicone, and latex.

In some examples, the double-layer balloon may maintain a small contracted volume when not under pressure. In some examples, the double-layer balloon may maintain a relatively small contracted volume when not under pressure. In some examples, the double-layer balloon may maintain a relatively small contracted volume when not under pressure compared to under pressure. In some examples, the double layer balloon may maintain a volume when under pressure. In some examples, the double layer balloon may maintain a stable volume when under pressure. In some examples, the double layer balloon may maintain a stable expanded volume when under pressure within a certain range. In some examples, the pressure range may be from about 0 atm to 40 atm. In some examples, the pressure range may be from about 0 atm to about 10 atm. In some examples, the pressure range may be from about 10 atm to about 20 atm. In some examples, the pressure range may be from about 20 atm to about 30 atm. In some examples, the pressure range may be from about 30 atm to about 40 atm. In some examples, the pressure range may be from about 0 atm to about 20 atm. In some examples, the double layer balloon may be configured to block the blood vessels at a designated location. In some examples, the double layer balloon may be configured to accurately block the blood vessels at the designated location. In some examples, the double layer balloon may be configured to not damage the blood vessels due to excessive expansion. In some examples, the double layer balloon may be configured to have puncture resistance. In some examples, the double layer balloon may be configured to have better puncture resistance. In some examples, the double layer balloon may be configured to have better puncture resistance, thereby reducing the risk of rupture.

In some examples, the inner ballon catheter may comprise a first hub. In some examples, the first hub may be connected to the first multi-lumen tube. In some examples, the first hub may be connected to an end of the first multi-lumen tube. In some examples, the first hub may be connected to the proximal end of the first multi-lumen tube. In some examples, the first hub may be connected to the distal end of the first multi-lumen tube.

In some examples, the first hub may comprise a first connector. In some examples, the first connector may be connected to a lumen. In some examples, the first connector may be connected to the first lumen. In some examples, the first connector may be configured to penetrate a guidewire, wherein the first lumen and first single-lumen tube may be configured to aid in guidewire use.

In some examples, the first hub may comprise a second connector. In some examples, the second connector may be connected to a lumen. In some examples, the second connector may be connected to the second lumen. In some examples, the second connector may be configured to convey a pressurized medium, wherein the second lumen may be configured to convey the pressurized medium.

In some examples, the inner balloon catheter may further comprise a developing element. In some examples, the inner balloon catheter may comprise a first developing element. In some examples, the first developing element may be disposed on the outer wall. In some examples, the first developing element may be disposed on the outer wall of a lumen. In some examples, the first developing element may be disposed on the outer wall of the first single-lumen tube. In some examples, the first developing element may be disposed in the first balloon.

In some examples, the developing element may be detected before surgery. In some examples, the developing element may be detected during surgery. In some examples, the developing element may be detected after surgery. In some examples, the developing element may be detected by an external image detection device (such as an X-ray detection device). In some examples, the detection of the developing element may be configured to accurately track the developing element position. In some examples, the detection of the developing element may be configured to achieve accurate positioning of instrument intervention. In some examples, the developing element may comprise a developing element material. In some examples, the developing element material may be a metal, metalloid, or non-metal. In some examples, the developing element material may be gold, platinum, iridium, tantalum, tungsten, or any other acceptable metal or a combination thereof.

In some examples, the catheter device may comprise an outer ballon catheter. In some examples, the inner ballon catheter may comprise a second multi-lumen tube. In some examples, the inner ballon catheter may comprise a second single-lumen tube. In some examples, the inner ballon catheter may comprise a second balloon. In some examples, the inner ballon catheter may comprise a second hub.

In some examples, the outer balloon catheter may comprise a second multi-lumen tube. In some examples, the second multi-lumen tube may comprise a more than one lumen. In some examples, the second multi-lumen tube may comprise two lumens, three lumens, four lumens, five lumens, or six or more lumens. In some examples, the second multi-lumen tube may comprise a first lumen. In some examples, the second multi-lumen tube may comprise a second lumen. In some examples, the second multi-lumen tube may comprise a third lumen. In some examples, the second multi-lumen tube may comprise a fourth lumen. In some examples, the lumens of the multi-lumen tube may be orthogonal. In some examples, the lumens of the multi-lumen tube may be parallel. In some examples, the lumens of the multi-lumen tube may be co-planar. In some examples, the lumens of the multi-lumen tube may be not in fluid connection. In some examples, the lumens of the multi-lumen tube may be in fluid connection.

In some examples, the outer ballon catheter may comprise a second single-lumen tube. In some examples, the second single-lumen tube may comprise a third lumen. In some examples, the second single-lumen tube may be connected to a third lumen. In some examples, the second single-lumen tube may be connected to the distal end of the third lumen. In some examples, the second single-lumen tube may be connected to the proximal end of the third lumen.

In some examples, the second single-lumen tube may comprise a body portion. In some examples, the second single-lumen tube may comprise a tip portion. In some examples, the tip portion may be connected to the body portion. In some examples, the tip portion may be connected to the end of the body portion. In some examples, the tip portion may be connected to the distal end of the body portion. In some examples, the tip portion may be connected to the proximal end of the body portion.

In some examples, the tip portion may be welded to the body portion. In some examples, the tip portion may be adhered to the body portion. In some examples, the tip portion may comprise metal, non-metal, plastic, or polymer. In some examples, the tip portion may include polyamide. In some examples, the tip portion may include polyurethane. In some examples, the tip portion may include polyether block polyamide. In some examples, the tip portion may include silicone. In some examples, the body portion may comprise metal, non-metal, plastic, or polymer. In some examples, the body portion may include polyamide. In some examples, the body portion may include polyurethane. In some examples, the body portion may include polyether block polyamide. In some examples, the body portion may include silicone. In some examples, the body portion may be of harder material than the tip portion. In some examples, the tip portion may be of softer material than the body portion. In some examples, the tip portion material may be configured to reduce the resistance of the catheter device. In some examples, the tip portion material may be configured to reduce the resistance of the catheter device in the blood vessel, to enable smoother entry. In some examples, the tip portion shape may be configured to reduce the resistance of the catheter device. In some examples, the tip portion shape may be configured to reduce the resistance of the catheter device in the blood vessel, to enable smoother entry.

In some examples, the outer ballon catheter may comprise a second balloon. In some examples, the second balloon may be connected to the second multi-lumen tube. In some examples, the second balloon may be connected to the outer wall of the second multi-lumen tube. In some examples, the second balloon may be connected to the outer wall of the second multi-lumen tube, wherein a seal is formed. In some examples, the second balloon may be connected to the second single-lumen tube. In some examples, the second balloon may be connected to the outer wall of the second single-lumen tube. In some examples, the second balloon may be connected to the outer wall of the second single-lumen tube, wherein a seal is formed. In some examples, the second balloon may be connected to a fourth lumen. In some examples, the second balloon may be connected to an end of the fourth lumen. In some examples, the second balloon may be connected to the distal end of the fourth lumen. In some examples, the second balloon may be connected to the proximal end of the fourth lumen.

In some examples, the second balloon may be a single layer balloon. In some examples, the second balloon may be a multi-layered balloon. In some examples, the second balloon may be a double layer balloon. In some examples, the second balloon may be a triple layer balloon. In some examples, the double layer second balloon may have an outer layer material. In some examples, the double layer second balloon may have an inner layer material. In some examples, the inner layer material and the outer layer material may be connected. In some examples, the inner layer material and the outer layer material may be attached to one another. In some examples, the inner layer material and the outer layer material may have a relative hardness. In some examples, the relative hardness of the inner layer material may be equal to the relative hardness of the outer layer material. In some examples, the relative hardness of the inner layer material may be less than the relative hardness of the outer layer material. In some examples, the relative hardness of the inner layer material may be greater than the relative hardness of the outer layer material.

In some examples, the inner layer material may comprise a metal. In some examples, the inner layer material may comprise a non-metal. In some examples, the inner layer material may comprise a polymer. In some examples, the inner layer material may comprise a plastic. In some examples, the inner layer material may comprise polyamide. In some examples, the inner layer material may comprise polyether block polyamide. In some examples, the inner layer material may comprise polyethylene terephthalate. In some examples, the inner layer material may comprise polyurethane. In some examples, the inner layer material may comprise thermoplastic elastomer. In some examples, the inner layer material may comprise silicone. In some examples, the inner layer material may comprise latex. In some examples, the inner layer material may comprise at least one of polyamide, polyether block polyamide, or polyethylene terephthalate.

In some examples, the outer layer material may comprise a metal. In some examples, the outer layer material may comprise a non-metal. In some examples, the outer layer material may comprise a polymer. In some examples, the outer layer material may comprise a plastic. In some examples, the outer layer material may comprise polyamide. In some examples, the outer layer material may comprise polyether block polyamide. In some examples, the outer layer material may comprise polyethylene terephthalate. In some examples, the outer layer material may comprise polyurethane. In some examples, the outer layer material may comprise thermoplastic elastomer. In some examples, the outer layer material may comprise silicone. In some examples, the outer layer material may comprise latex. In some examples, the outer layer material may comprise at least one of polyether block polyamide, polyurethane, thermoplastic elastomer, silicone, and latex.

In some examples, the double-layer balloon may maintain a small contracted volume when not under pressure. In some examples, the double-layer balloon may maintain a relatively small contracted volume when not under pressure. In some examples, the double-layer balloon may maintain a relatively small contracted volume when not under pressure compared to under pressure. In some examples, the double layer balloon may maintain a volume when under pressure. In some examples, the double layer balloon may maintain a stable volume when under pressure. In some examples, the double layer balloon may maintain a stable expanded volume when under pressure within a certain range. In some examples, the pressure range may be from about 0 atm to 40 atm. In some examples, the pressure range may be from about 0 atm to about 10 atm. In some examples, the pressure range may be from about 10 atm to about 20 atm. In some examples, the pressure range may be from about 20 atm to about 30 atm. In some examples, the pressure range may be from about 30 atm to about 40 atm. In some examples, the pressure range may be from about 0 atm to about 20 atm. In some examples, the double layer balloon may be configured to block the blood vessels at a designated location. In some examples, the double layer balloon may be configured to accurately block the blood vessels at the designated location. In some examples, the double layer balloon may be configured to not damage the blood vessels due to excessive expansion. In some examples, the double layer balloon may be configured to have puncture resistance. In some examples, the double layer balloon may be configured to have better puncture resistance. In some examples, the double layer balloon may be configured to have better puncture resistance, thereby reducing the risk of rupture.

In some examples, the outer ballon catheter may be in connection to a second hub. In some examples, the second hub may be connected to the second multi-lumen tube. In some examples, the second hub may be connected to an end of the second multi-lumen tube. In some examples, the second hub may be connected to the proximal end of the second multi-lumen tube. In some examples, the second hub may be connected to the distal end of the second multi-lumen tube.

In some examples, the second hub may in connection to a third connector. In some examples, the third connector may be connected to a lumen. In some examples, the third connector may be connected to the third lumen. In some examples, the third connector may be configured to penetrate an inner balloon catheter, wherein the third lumen and second single-lumen tube may be configured to aid in inner balloon catheter use.

In some examples, the second hub may be in connection to a fourth connector. In some examples, the fourth connector may be connected to a lumen. In some examples, the fourth connector may be connected to the fourth lumen. In some examples, the fourth connector may be configured to transport a pressurized medium, wherein the fourth lumen may be configured to transport the pressurized medium.

In some examples, the second hub may be in connection to a locking structure. In some examples, the locking structure may be connected to a connector. In some examples, the locking structure may be connected to the third connector. In some examples, the end of the locking structure may be connected to the third connector. In some examples, the proximal end of the locking structure may be connected to the third connector. In some examples, the distal end of the locking structure may be connected to the third connector. In some examples, the locking structure may be configured to lock the relative sliding position of the inner balloon catheter in the outer balloon catheter. In some examples, the locking structure may have structural form for its purpose. In some examples, the locking structure may be simple in design. In some examples, the locking structure may be easy to operate. In some examples, the locking structure may achieve reliable locking.

In some examples, the locking structure may comprise a thread segment. In some examples, the locking structure may comprise an internal thread segment. In some examples, the locking structure may comprise an external thread segment. In some examples, the external thread segment may be positioned on the third connector. In some examples, the external thread segment may be positioned at the end of the third connector. In some examples, the external thread segment may be positioned at the proximal end of the third connector. In some examples, the external thread segment may be positioned at the distal end of the third connector.

In some examples, the locking structure may comprise a gland. In some examples, the gland may comprise an internal thread segment. In some examples, the gland may comprise an external thread segment. In some examples, internal thread segment mat be connected to the external thread segment of the locking structure.

In some examples, the locking structure may comprise a valve. In some examples, the locking structure may comprise a hemostatic valve. In some examples, the hemostatic valve may be provided on the external thread segment. In some examples, the hemostatic valve may be provided on the inner side of the external thread segment. In some examples, the hemostatic valve may have a through-hole. In some examples, the through-hole may be configured to allow for the inner balloon catheter to pass through. In some examples, the hemostatic valve may have its own elastic force. In some examples, the hemostatic valve may use its own elastic force to closely contact with other components. In some examples, the hemostatic valve may be configured to squeeze and seal to prevent blood from flowing out. In some examples, the hemostatic valve may be configured to squeeze and seal to achieve a hemostatic effect. In some examples, the hemostatic valve may comprise valve material. In some examples, the hemostatic material may comprise metal or non-metal. In some examples, the hemostatic material may comprise a polymer. In some examples, the hemostatic material may comprise a plastic. In some examples, the hemostatic material may comprise silicone. In some examples, the hemostatic material may comprise latex. In some examples, the hemostatic material may comprise polyurethane.

In some examples, the hemostatic valve may press the inner balloon catheter. In some examples, the hemostatic valve may press the inner balloon catheter by tightening the gland. In some examples, the hemostatic valve may press the inner balloon catheter to lock the relative sliding position in the outer balloon catheter.

In some examples, the outer balloon catheter may further comprise a developing element. In some examples, the outer balloon catheter may further comprise a second developing element. In some examples, the second developing element may be disposed on the outer wall. In some examples, the second developing element may be disposed on the outer wall of a lumen. In some examples, the second developing element may be disposed on the outer wall of the second single-lumen tube. In some examples, the first developing element may be disposed in the second balloon.

In some examples, the developing element may be detected before surgery. In some examples, the developing element may be detected during surgery. In some examples, the developing element may be detected after surgery. In some examples, the developing element may be detected by an external image detection device (such as an X-ray detection device). In some examples, the detection of the developing element may be configured to accurately track the developing element position. In some examples, the detection of the developing element may be configured to achieve accurate positioning of instrument intervention. In some examples, the developing element may comprise a developing element material. In some examples, the developing element material may be a metal, metalloid, or non-metal. In some examples, the developing element material may be gold, platinum, iridium, tantalum, tungsten, or any other acceptable metal or a combination thereof.

In some examples, the catheter device may comprise a hub. In some examples, the inner balloon catheter may comprise a hub. In some examples, the outer balloon catheter may comprise a hub. In some examples, the catheter device may comprise a first hub and a second hub. In some examples, the hub may comprise hub material. In some examples, the hub material may comprise a metal. In some examples, the hub material may comprise a non-metal. In some examples, the hub material may comprise plastic. In some examples, the hub material may comprise polymer. In some examples, the hub material may comprise polyamide. In some examples, the hub material may comprise polycarbonate. In some examples, the hub material may comprise polyoxymethylene.

In some examples, the catheter device may comprise a connector, or a connection seat. In some examples, the connector, or seat, may provide an inlet channel. In some examples, the connector, or seat, may provide an inlet channel for a guide wire. In some examples, the connector, or seat, may provide an inlet channel for a catheter. In some examples, the connector, or seat, may provide an inlet channel for a treatment-related medium. In some examples, the connector, or seat, may provide an outlet channel. In some examples, the connector, or seat, may provide an outlet channel for a guidewire. In some examples, the connector, or seat, may provide an outlet channel for a catheter. In some examples, the connector, or seat, may provide an outlet channel for a treatment-related medium. In some examples, the connector, or seat, may be configured for valve installation. In some examples, the connector, or seat, may be configured for hemostatic valve installation. In some examples, the connector, or seat, may comprise a material. In some examples, the connector material may be a metal, non-metal, plastic, polymer, or any suitable material. In some examples, the connector material is hard relative to other components of the device, such as the balloon, lumen, or hub. In some examples, the connector material may be configured to allow for ease of assembly. In some examples, the connector material may be configured to allow for ease of instrument operation.

In some examples, each lumen may have a cross sectional shape. In some examples, the cross sectional shape may be any suitable shape. In some examples, the cross sectional shape may be circular, semi-circular, crescent, rectangular, or any other shape. In some examples, the cross sectional shape of the first lumen may be circular. In some examples, the cross sectional shape of the second lumen may be crescent-shaped. In some examples, the cross sectional shape of the third lumen may be circular. In some examples, the cross sectional shape of the fourth lumen may be crescent-shaped.

In some examples, each lumen may comprise lumen material. In some examples, the lumen material may comprise metal. In some examples, the lumen material may comprise non-metal. In some examples, the lumen material may comprise polymer. In some examples, the lumen material may comprise plastic. In some examples, the lumen material may comprise polyamide. In some examples, the lumen material may comprise polyether block polyamide. In some examples, the lumen material may comprise polyurethane. In some examples, the lumen material may comprise polyethylene. In some examples, the lumen material may comprise polypropylene. In some examples, the lumen material may comprise at least one of polyamide, polyether block polyamide, polyurethane, polyethylene, and polypropylene. In some examples, the first multi-lumen tube may comprise lumen material. In some examples, the second multi-lumen tube may comprise lumen material.

In some examples, the first lumen may have a central axis. In some examples, the central axis may be the center location of the first lumen cross section. In some examples, the central axis of the first lumen may be located within the first multi-lumen tube. In some examples, the central axis of the first lumen may be eccentrically located within the first multi-lumen tube. In some examples, the central axis of the first lumen may be centrally located within the first multi-lumen tube. In some examples, the central axis of the first lumen may be eccentrically located to the central axis of the first multi-lumen tube. In some examples, the central axis of the first lumen may be centrally located to the central axis of the first multi-lumen tube. In some examples, the first lumen may be configured to facilitate the passage of a circular guide wire.

In some examples, the third lumen may have a central axis. In some examples, the central axis may be the center location of the third lumen cross section. In some examples, the central axis of the third lumen may be located within the second multi-lumen tube. In some examples, the central axis of the third lumen may be eccentrically located within the second multi-lumen tube. In some examples, the central axis of the third lumen may be centrally located within the second multi-lumen tube. In some examples, the central axis of the third lumen may be eccentrically located to the central axis of the second multi-lumen tube. In some examples, the central axis of the third lumen may be centrally located to the central axis of the second multi-lumen tube. In some examples, the third lumen may facilitate the passage of the inner balloon catheter with a circular cross section outer contour, wherein the outer contour may allow for entry to be smooth.

In some examples, the catheter device may further comprise a reinforcing sleeve. In some examples, the reinforcing sleeve may be provided at the connection between the first multi-lumen tube and the first hub. In some examples, the reinforcing sleeve may seal the first multi-lumen tube to the first hub. In some examples, the reinforcing sleeve may be used to seal the connection between the first multi-lumen tube and the first hub firmly. In some examples, the reinforcing sleeve may prevent excessive bending at the connection. In some examples, the reinforcing sleeve may comprise a material which may include at least one of a polyamide, an acrylonitrile-butadiene-styrene terpolymer, a polyolefin, a polyether block polyamide, and the like, and may also include a metal oxide such as barium sulfate.

In some examples, the outer balloon catheter may be configured to enter a subject. In some examples, the outer balloon catheter may be configured to enter a human body. In some examples, the outer balloon catheter may be configured to enter the inferior vena cava. In some examples, the outer balloon catheter may be introduced into the human body under the guidance of the guide kit. In some examples, the outer balloon catheter may be introduced into the femoral vein under the guidance of the guide kit. In some examples, the second balloon of the outer balloon catheter may be positioned in the inferior vena cava. In some examples, the second balloon of the outer balloon catheter may be positioned in the inferior vena cava under angiography. In some examples, the second balloon of the outer balloon catheter may be positioned below the renal vein.

In some examples, the inner balloon catheter may be configured to pass through the outer balloon catheter. In some examples, the inner balloon catheter may be configured to pass along the vein to enter the subclavian vein. In some examples, the inner balloon catheter may be introduced into the third connector under the guidance of the guide kit. In some examples, the inner balloon catheter may be introduced into the third lumen under the guidance of the guide kit. In some examples, the inner balloon catheter may be introduced into the second single lumen under the guidance of the guide kit. In some examples, the inner balloon catheter may be introduced into the outer balloon catheter. In some examples, the second balloon of the outer balloon catheter may be positioned in the subclavian vein. In some examples, the second balloon of the outer balloon catheter may be positioned in the subclavian vein along the venous blood vessels. In some examples, the first balloon of the inner balloon catheter may be positioned in the subclavian vein under angiography.

In some examples, the positions of the inner balloon catheter and the outer balloon catheter may be locked. In some examples, the positions of the inner balloon catheter and the outer balloon catheter may be locked. In some examples, the positions of the inner balloon catheter and the outer balloon catheter may be locked by the locking structure. In some examples, when their relative positions are locked, the inner balloon catheter and the outer balloon catheter may be connected to the pressure pumps.

In some examples, when their relative positions are locked, the inner balloon catheter and the outer balloon catheter may be connected to the pressure pumps to allow for treatment to begin. In some examples, the treatment may comprise filling the inner balloon catheter and the outer balloon catheter. In some examples, the treatment may comprise blocking the superior vena cava blood with the inner balloon catheter and the outer balloon catheter. In some examples, the treatment may comprise blocking the inferior vena cava blood with the inner balloon catheter and the outer balloon catheter.

In some examples, the balloons of the inner and outer balloon catheters may be configured to block the veins. In some examples, the balloons of the inner and outer balloon catheters may be configured to intermittently block the veins. In some examples, the balloons of the inner and outer balloon catheters may be configured to reduce the venous blood backflow. In some examples, the balloons of the inner and outer balloon catheters may be configured to reduce the venous blood backflow to the heart. In some examples, the balloons of the inner and outer balloon catheters may be configured to reduce the precardiac load. In some examples, the balloons of the inner and outer balloon catheters may be configured to reduce ventricular wall stress.

In some examples, the balloons of the inner and outer balloon catheters may be configured to treat heart failure. In some examples, the balloons of the inner and outer balloon catheters may be configured to treat acute congestive heart failure. In some examples, the balloons of the inner and outer balloon catheters may be configured to treat acute congestive heart failure with a good prognosis.

In some examples, the catheter device may be used with a guide kit. In some examples, the catheter device may be used with the aid of a guide kit. In some examples, the guide kit may comprise a puncture needle. In some examples, the guide kit may comprise a sheath. In some examples, the guide kit may comprise a guide wire. In some examples, the guide kit may comprise any suitable guide components.

In some examples, the catheter device may be a component of a heart failure treatment device. In some examples, the heart failure treatment device may comprise a guide kit. In some examples, the heart failure treatment device may comprise a pressure pump. In some examples, the heart failure treatment device may comprise a pressure detection device. In some examples, the heart failure treatment device may be configured for venous occlusion treatment. In some examples, the heart failure treatment device may be configured for venous occlusion treatment scheme, so that a good prognosis can be obtained in the treatment of acute congestive heart failure.

### Methods of Use

In some examples, methods of use of the catheter instrument may be disclosed. In some examples, the catheter instrument may be configured to intermittently block veins. In some examples, the catheter instrument may be configured to use the balloon of the catheter instrument to intermittently block veins. In some examples, the catheter instrument may be configured to pass the balloon of the balloon catheter through the catheterization device. In some examples, the catheter instrument may be configured to reduce the return of venous blood to the heart. In some examples, the catheter instrument may be configured to decrease the preload on the heart. In some examples, the catheter instrument may be configured to allow for a reduction in ventricular wall stress. In some examples the instrument may be passed through the lumen to the diagnostic device during treatment. In some examples, the instrument may be passed through the lumen to the diagnostic device before treatment. In some examples, the instrument may be passed through the lumen to the diagnostic device after treatment. In some examples, the instrument may be configured for relevant testing and treatment. In some examples, the catheter instrument may be configured to monitor the intravascular pressure through the pressure-monitoring lumen. In some examples, the catheter instrument may be used in the treatment of acute congestive heart failure, and can obtain a good prognosis.

In some examples, the catheter instrument may be positioned in a subject. In some examples, the subject may be a mammal. In some examples, the subject may be a human. In some examples, the catheter instrument may be positioned in a human body. In some examples, the catheter instrument may be positioned before the balloon catheter is inserted into the subject. In some examples, the catheter instrument may be positioned through the femoral vein. In some examples, insertion of the catheter instrument may include performing a puncture to the subject. In some examples, insertion of the catheter instrument may include performing a puncture using a puncture needle. In some examples, insertion of the catheter instrument may include placing a short guidewire in the puncture needle. In some examples, insertion of the catheter instrument may include withdrawing the puncture needle. In some examples, insertion of the catheter instrument may include placing the catheterization device using the short guidewire as a guide. In some examples, insertion of the catheter instrument may include withdrawing the short guidewire. In some examples, insertion of the catheter instrument may include placing a long guidewire into the balloon catheter of the catheterization device through the lumen. In some examples, insertion of the catheter instrument may include placing the balloon catheter using the long guidewire as a guide. In some examples, insertion of the catheter instrument may include withdrawing the long guidewire.

In some examples, the insertion of the catheter instrument may include use of a guidewire. In some examples, the guidewire may be a short guidewire. In some examples, the guidewire may be a long guidewire. In some examples, the guidewire may be used as an intermediate instrument when placing the catheter instrument. In some examples, the guidewire may be used as an intermediate instrument when placing the balloon catheter. In some examples, the guidewire may be used to advance a catheter through vasculature. In some examples, the guidewire may be configured to position a first balloon of the catheter within the inferior vena cava by advancing the catheter through vasculature of the subject over a guidewire. In some examples, the guidewire may be configured to position a second balloon at or near the subclavian vein of the subject by advancing the catheter through vasculature of the subject over a guidewire. In some examples, the guidewire may be configured to position a third balloon at or near the subclavian vein of the subject by advancing the catheter through vasculature of the subject over a guidewire.

In some examples, the instrument may be passed through the lumen. In some examples, the instrument may be passed through the lumen during treatment. In some examples, the instrument may be passed through the lumen before treatment. In some examples, the instrument may be passed through the lumen after treatment. In some examples, the instrument may be passed for relevant testing. In some examples, the instrument may be passed for treatment. In some examples, the instrument may be passed for pressure monitoring. In some examples, the instrument may be positioned in the inferior vena cava (IVC). In some examples, the instrument may be positioned near the renal vein. In some examples, the instrument may be positioned near the subclavian vein.

In some examples, the catheter instrument may be a component of a heart failure treatment system. In some examples, the heart failure treatment system may be applied through the femoral artery. In some examples, the heart failure treatment system application may comprise establishing a channel in the femoral artery. In some examples, the channel may be established by a puncture kit. In some examples, the heart failure treatment system application may comprise placing a guidewire in the channel. In some examples, the heart failure treatment system application may comprise introducing the catheter instrument into the body along the guidewire from the femoral vein. In some examples, the heart failure treatment system application may comprise positioning the balloon under contrast to the inferior vena cava, under the renal vein. In some examples, the heart failure treatment system application may comprise positioning the balloon to secure the catheter instrument. In some examples, the heart failure treatment system application may comprise placing a guidewire in the balloon catheter through the lumen. In some examples, the guidewire may be passed upwardly along the inferior vena cava, superior vena cava, head/arm vein, and to the subclavian vein. In some examples, the heart failure treatment system application may comprise placing the balloon catheter to reach a designated position along the guidewire. In some examples, the heart failure treatment system application may comprise delivering a contrast agent to the blood vessel through contrast agent delivery holes within the blood catheter to accurately reach the blocking location. In some examples, the heart failure treatment system application may comprise connecting the catheter instrument and balloon catheter to the pressure pump once positioned.

In some examples, venous occlusion treatment may comprise occluding the veins. In some examples, venous occlusion may take place in the IVC. In some examples, venous occlusion in the IVC may create a low-pressure area at the renal vein junction. In some examples, venous occlusion may take place in the subclavian vein. In some examples, venous occlusion in the subclavian vein may create a low-pressure area at the thoracic duct. In some examples, vein occlusion may be the result of two balloons working together. In some examples, the two balloons may be configured to reduce the venous return to the heart and reducing the cardiac preload. In some examples, venous occlusion treatment may comprise synchronous inflation and occlusion of the two balloons. In some examples, venous occlusion may comprise asynchronous inflation and occlusion of the two balloons. In some examples, intermittent venous occlusion treatment may comprise synchronous inflation and occlusion of the two balloons. In some examples, intermittent venous occlusion may comprise asynchronous inflation and occlusion of the two balloons.

In some examples, creation of a low-pressure area may have effects. In some examples, creation of a low-pressure area may minimize hemodynamic disturbance. In some examples, minimization of hemodynamic disturbance may be characterized by maintenance of stable blood pressure, superior vena cava pressure, or inferior vena cava pressure. In some examples, minimization of hemodynamic disturbance may be characterized by a variation in pressure. In some examples, the pressure variation may be less than 50 mmHg, 45 mmHg, 40 mmHg, 35 mmHg, 30 mmHg, 25 mmHg, 20 mmHg, 15 mmHg, 10 mmHg, or 5 mmHg. In some examples, the pressure variation may be less than 20 mmHg In some examples, creation of a low-pressure area may achieve optimal therapeutic effects. In some examples, optimal therapeutic effect may be characterized by stabilization of hemodynamic disturbances. In some examples, creation of a low-pressure area may achieve optimal therapeutic effects for acute heart failure. In some examples, creation of a low-pressure area may reduce pulmonary artery pressure. In some examples, creation of a low-pressure area may demonstrate favorable acute and long-term safety. In some examples, favorable acute safety may be demonstrated by hemodynamic stability. In some examples, favorable long-term safety may be demonstrated by development of controllable vascular damage or thrombosis.

In some examples, the catheter body, or tube, may be configured for insertion to a subject. In some examples, insertion of the catheter body, or tube, may comprise using a puncture needle to perform puncture. In some examples, insertion of the catheter body, or tube, may comprise inserting a short guide wire into the puncture needle. In some examples, insertion of the catheter body, or tube, may comprise pulling out the puncture needle. In some examples, insertion of the catheter body, or tube, may comprise inserting the sheath tube with the short guide wire as a guide. In some examples, insertion of the catheter body, or tube, may comprise pulling out the short guide wire. In some examples, insertion of the catheter body, or tube, may comprise inserting a long guide wire with the sheath tube as a guide. In some examples, insertion of the catheter body, or tube, may comprise inserting the catheter device with the long guide wire as a guide (the long guide wire passes through the first lumen) and positioning it (with the help of external imaging detection equipment). In some examples, insertion of the catheter body, or tube, may comprise pulling out the long guide wire.

In some examples, the balloon sheath may be configured for insertion into a subject. In some examples, the balloon sheath may be inserted into a subject as a part of an operation. In some examples, the operation process may comprise placing the balloon sheath tube and the balloon catheter in the human body. In some examples, the operation process may comprise performing a puncture using a puncture needle. In some examples, the operation process may comprise placing a short guidewire in the puncture needle. In some examples, the operation process may comprise pulling out the puncture needle. In some examples, the operation process may comprise placing the balloon sheath tube guided by the short guidewire. In some examples, the operation process may comprise pulling out the short guide wire and the main dilator. In some examples, the operation process may comprise threading the long guide wire through the secondary lumen of the balloon sheath through the secondary dilator pre-positioned in the third port of the secondary connector seat. In some examples, the operation process may comprise pulling out the secondary dilator. In some examples, the operation process may comprise placing the long guide wire guided into the balloon catheter. In some examples, the operation process may comprise pulling out the long guide wire. Therein, the short guide wire, the long guide wire, the main dilator, and the sub-dilator serve as intermediate auxiliary instruments when placing the balloon sheath tube and the balloon catheter.

In some examples, the balloon sheath may be a component of a heart failure treatment device, wherein the device may be used to implement treatment. In some examples, a channel may be established in the femoral vein by means of a puncture kit. In some examples, a guidewire may be placed in the femoral artery. In some examples, a balloon sheath tube may be introduced into the body from the femoral vein along the guidewire, wherein the guidewire is threaded into the main lumen of the balloon sheath tube. In some examples, the first balloon may be positioned under contrast to the inferior vena cava, which is located underneath the renal vein. In some examples, positioning of the first balloon may secure the balloon sheath tube. In some examples, a guidewire may be placed in the secondary lumen of the balloon sheath tube, so that the guidewire passes upwardly along the inferior vena cava through the heart, the superior vena cava, and the cephalic arm vein to the subclavian vein. In some examples, the balloon catheter may be placed along the guidewire through the secondary lumen to reach the designated position. In some examples, a contrast agent may be delivered into the blood vessel while reaching the designated position through the contrast agent delivery holes, wherein the delivery holes are provided with the balloon catheter. In some examples, the contrast agent may be configured to monitor that the second balloon accurately arrives at the blocking position.

In some examples, the balloon sheath and balloon catheter may be connected to pressure pumps. In some examples, the pressure pumps may then begin a cyclic treatment process comprising four steps. In Step 1, the first balloon may complete pressure filling, and the pressure may be maintained while the second balloon remains unpressurized. In Step 2, the first balloon may be depressurized to complete pressure relief to allow blood flow. In Step 3, the second balloon may complete filling the pressure, and the pressure is maintained, at which time the first balloon is maintained in a non-pressurized state. In Step 4, the second balloon completes the pressure relief to allow the blood to flow, at which time the first balloon remains unpressurized. In some examples, the cyclic treatment may be completed once. In some examples, the cyclic treatment may be completed more than once. In some examples, the cyclic treatment cycle may be repeated until the end of treatment. In some examples, the cyclic treatment may result in a slowing of venous blood return to the heart. In some examples, the cyclic treatment may result in a reducing of preload of the heart. In some examples, the main lumen of the balloon sheath may be configured to be passed into a diagnostic instrument, or device before, during, or after the treatment process.

In some examples, the first balloon and the second balloon may be pressurized. In some examples, the pressurizing may take from about 1 second to about 20 seconds. In some examples, the pressurizing may take from about 1 second to about 5 seconds. In some examples, the pressurizing may take from about 6 second to about 10 seconds. In some examples, the pressurizing may take from about 10 second to about 15 seconds. In some examples, the pressurizing may take from about 15 second to about 20 seconds. In some examples, the pressurizing may take from about 4 seconds to about 5 seconds.

In some examples, the first balloon and the second balloon may have the pressure maintained. In some examples, the pressure maintenance may take from about 1 second to about 100 seconds. In some examples, the pressure maintenance may take from about 1 second to about 20 seconds. In some examples, the pressure maintenance may take from about 20 seconds to about 40 seconds. In some examples, the pressure maintenance may take from about 40 seconds to about 60 seconds. In some examples, the pressure maintenance may take from about 60 seconds to about 80 seconds. In some examples, the pressure maintenance may take from about 80 seconds to about 100 seconds. In some examples, the pressure maintenance may take about 15 seconds.

In some examples, the first balloon and the second balloon may be depressurized. In some examples, the depressurizing may take from about 1 second to about 20 seconds. In some examples, the depressurizing may take from about 1 second to about 5 seconds. In some examples, the depressurizing may take from about 6 second to about 10 seconds. In some examples, the depressurizing may take from about 10 second to about 15 seconds. In some examples, the depressurizing may take from about 15 second to about 20 seconds. In some examples, the depressurizing may take from about 4 seconds to about 5 seconds.

In some examples, the blood flow may be allowed to circulate. In some examples, the blood may circulate from around 30 seconds to around 10 minutes. In some examples, the blood may circulate from around 30 seconds to around 2 minutes. In some examples, the blood may circulate from around 2 minutes to around 4 minutes. In some examples, the blood may circulate from around 4 minutes to around 6 minutes. In some examples, the blood may circulate from around 6 minutes to around 8 minutes. In some examples, the blood may circulate from around 8 minutes to around 10 minutes. In some examples, the blood may circulate for around 1 minute.

In some examples, the balloon sheath may be a component of a heart failure treatment device, wherein the device may be used to implement treatment. In some examples, a channel may be established in the femoral artery by means of a puncture kit. In some examples, a guidewire may be placed in the femoral artery. In some examples, the balloon sheath tube may be introduced into the body from the femoral artery along the guidewire (which is threaded into the main lumen of the balloon sheath tube). In some examples, the first balloon may be positioned under the aortic arch under the angiography, which is located in the thoracic aorta, to secure the balloon sheath tube. In some examples, the collateral lumen of the balloon sheath tube may be placed with the guidewire, so that the guidewire enters one of the left subclavian artery, the common carotid artery, and the trunk artery of the head and arm along the femoral artery (depending on the clinical assessment). In some examples, the balloon catheter may be passed along the guidewire through the secondary lumen to reach the designated position, and in the process, the contrast agent may be delivered to the vessel through the contrast agent delivery holes that are provided with the balloon catheter, so as to monitor that the second balloon accurately arrives at the blockage position.

After the second balloon is positioned, the balloon sheath tube and the balloon catheter may be connected to pressure pumps, respectively, to start a treatment process. In some examples, the treatment process may comprise, when the heart is in diastole, filling and blocking the first balloon and the second balloon, and when the heart is in systole, delating the first balloon and the second balloon, resulting in a dual hemodynamic effect. In some examples, the treatment process may comprise filling the two balloons with pressure in diastole to bring the blood flow forward, which improves the diastolic blood pressure and coronary perfusion. In some examples, the treatment process may comprise deflating the two balloons in systole to reduce the systolic blood pressure (cardiac afterload), which improves left ventricular ejection. In some examples, the treatment process may comprise inflating and deflating the first balloon and the second balloon by the pressure pump once every cardiac cycle (according to the 1:1 mode), once every two cardiac cycles (according to the 1:2 mode), and once every three cardiac cycles (according to the 1:3 mode). In some examples, the main lumen of the balloon sheath may be configured to be passed into a diagnostic instrument, or device before, during, or after the treatment process.

In some examples, the heart failure device may be configured for treatment implementation. In some examples, the heart failure device may be configured to measure hemodynamic data, or parameter. In some examples, the device may be configured to measure heart rate, blood pressure, or cardiac output. In some examples, the device may be configured to measure pressure. In some examples, the device may measure the pressure at the subclavian vein. In some examples, the device may measure the pressure at the IVC. In some examples, the device may adjust the balloon inflation in response to the measured pressure at the IVC. In some examples, the device may balloon vein adjustment may modulate the pressure at the renal vein junction, wherein the modulation may be an increase or decrease. In some examples, the device may adjust the balloon inflation in response to the measured pressure at the IVC. In some examples, the device may balloon vein adjustment may modulate the pressure at the thoracic duct, wherein the modulation may be an increase or decrease.

In some examples, the heart failure device may be configured to monitor hemodynamic data, or parameter. In some examples, the device may be configured to monitor heart rate, blood pressure, or cardiac output. In some examples, the device may be configured to monitor pressure at an artery. In some examples, the device may be configured to monitor pressure at one or more of an inferior vena cava or femoral vein.

In some examples, the treatment may comprise introducing the outer balloon catheter. In some examples, the treatment may comprise introducing the outer balloon catheter under the guidance of the guide kit. In some examples, the treatment may comprise introducing the outer balloon catheter into the human body. In some examples, the treatment may comprise introducing the outer balloon catheter into the human body from the femoral vein.

In some examples, the treatment may comprise positioning the second balloon. In some examples, the treatment may comprise positioning the second balloon in the inferior vena cava. In some examples, the treatment may comprise positioning the second balloon in the inferior vena cava under angiography. In some examples, the treatment may comprise positioning the second balloon in the inferior vena cava below the renal vein.

In some examples, the treatment may comprise positioning the inner balloon catheter. In some examples, the treatment may comprise positioning the inner balloon catheter under the guidance of the guide kit. In some examples, the treatment may comprise passing the inner balloon catheter through the third connector. In some examples, the treatment may comprise passing the inner balloon catheter through the third lumen. In some examples, the treatment may comprise passing the inner balloon catheter through the second single-lumen tube. In some examples, the treatment may comprise passing the inner balloon catheter through the outer balloon catheter. In some examples, the treatment may comprise passing the inner balloon catheter through the outer balloon catheter so that the first balloon is positioned in the subclavian vein under angiography. In some examples, the treatment may comprise passing the inner balloon catheter through the outer balloon catheter so the relative sliding position of the inner balloon catheter in the outer balloon catheter is locked by the locking structure.

In some examples, once the inner and outer balloon catheter positions are locked, the outer balloon catheter and the inner balloon catheter may be connected to the pressure pumps respectively. In some examples, once the inner and outer balloon catheter positions are locked, the cyclic treatment process may commence.

In some examples, once the inner and outer balloon catheter positions are locked, the cyclic treatment process may commence in four steps. In Step 1, the second balloon may complete pressure filling, and the pressure may be maintained while the first balloon remains unpressurized. In Step 2, the second balloon may be depressurized to complete pressure relief to allow blood flow. In Step 3, the first balloon may complete filling the pressure, and the pressure is maintained, at which time the second balloon may be maintained in a non-pressurized state. In Step 4, the first balloon completes the pressure relief to allow the blood to flow, at which time the second balloon remains unpressurized. In some examples, the cyclic treatment may be completed once. In some examples, the cyclic treatment may be completed more than once. In some examples, the cyclic treatment cycle may be repeated until the end of treatment. In some examples, the cyclic treatment may result in a slowing of venous blood return to the heart. In some examples, the cyclic treatment may result in a reducing of preload of the heart.

In some examples, the first balloon and the second balloon may be pressurized. In some examples, the pressurizing may take from about 1 second to about 20 seconds. In some examples, the pressurizing may take from about 1 second to about 5 seconds. In some examples, the pressurizing may take from about 6 second to about 10 seconds. In some examples, the pressurizing may take from about 10 second to about 15 seconds. In some examples, the pressurizing may take from about 15 second to about 20 seconds. In some examples, the pressurizing may take from about 4 seconds to about 5 seconds.

In some examples, the first balloon and the second balloon may have the pressure maintained. In some examples, the pressure maintenance may take from about 1 second to about 100 seconds. In some examples, the pressure maintenance may take from about 1 second to about 20 seconds. In some examples, the pressure maintenance may take from about 20 seconds to about 40 seconds. In some examples, the pressure maintenance may take from about 40 seconds to about 60 seconds. In some examples, the pressure maintenance may take from about 60 seconds to about 80 seconds. In some examples, the pressure maintenance may take from about 80 seconds to about 100 seconds. In some examples, the pressure maintenance may take about 15 seconds.

In some examples, the first balloon and the second balloon may be depressurized. In some examples, the depressurizing may take from about 1 second to about 20 seconds. In some examples, the depressurizing may take from about 1 second to about 5 seconds. In some examples, the depressurizing may take from about 6 second to about 10 seconds. In some examples, the depressurizing may take from about 10 second to about 15 seconds. In some examples, the depressurizing may take from about 15 second to about 20 seconds. In some examples, the depressurizing may take from about 4 seconds to about 5 seconds.

In some examples, the blood flow may be allowed to circulate. In some examples, the blood may circulate from around 30 seconds to around 10 minutes. In some examples, the blood may circulate from around 30 seconds to around 2 minutes. In some examples, the blood may circulate from around 2 minutes to around 4 minutes. In some examples, the blood may circulate from around 4 minutes to around 6 minutes. In some examples, the blood may circulate from around 6 minutes to around 8 minutes. In some examples, the blood may circulate from around 8 minutes to around 10 minutes. In some examples, the blood may circulate for around 1 minute.

In some examples, the catheter device may be configured for treatment of venous occlusion. In some examples, the treatment may comprise blocking the veins. In some examples, the treatment may comprise blocking the veins using a single balloon. In some examples, the treatment may comprise blocking the veins using two balloons. In some examples, the treatment may comprise blocking the veins using two balloons in combination. In some examples, the treatment may comprise blocking the veins using two balloons in combination with inflation. In some examples, the treatment may comprise blocking the veins using two balloons in combination with decompression. In some examples, the treatment may comprise blocking the veins using two balloons in combination with intermittent inflation and decompressions. In some examples, the treatment may comprise blocking the veins using two balloons in combination, wherein the return of blood may be reduced. In some examples, the treatment may comprise blocking the veins using two balloons in combination, wherein the return of blood from the veins to the heart may be reduced. In some examples, the treatment may comprise blocking the veins using two balloons in combination, alleviating the precardiac load. In some examples, the treatment may comprise blocking the veins using two balloons in combination, achieving a good prognosis.

In some examples, the catheter device may be used in the human body for a period of time. In some examples, the catheter device may be used in the human body from about 10 minutes to about 24 hours. In some examples, the catheter device may be used in the human body from about 10 minutes to about 4 hours. In some examples, the catheter device may be used in the human body from about 4 hours to about 8 hours. In some examples, the catheter device may be used in the human body from about 8 hours to about 12 hours. In some examples, the catheter device may be used in the human body from about 12 hours to about 16 hours. In some examples, the catheter device may be used in the human body from about 16 hours to about 20 hours. In some examples, the catheter device may be used in the human body from about 20 hours to about 24 hours.

In some examples, the catheter device may be utilized in a sterile environment. In some examples, the catheter device may be used in a non-sterile environment. In some examples, the catheter device may be equipped with a sheath for non-sterile environments. In some examples, the catheter device may be equipped with a vascular sheath for non-sterile environments. In some examples, the vascular sheath may be coaxially sleeved. In some examples, the vascular sheath may be sleeved on the outside of the second multi-lumen tube of the outer balloon catheter. In some examples, the vascular sheath may be coaxially sleeved on the outside of the second multi-lumen tube of the outer balloon catheter. In some examples, the vascular sheath may comprise a sheath tube. In some examples, the vascular sheath may comprise a seat.

In some examples, the catheter device may be equipped with a sterile sheath for non-sterile environments. In some examples, the sterile sheath may comprise more than one end. In some examples, the sterile sheath may be connected to the seat of the vascular sheath on a first end. In some examples, the sterile sheath may be connected to the second hub of the outer balloon catheter on a second end. In some examples, the sterile sheath length may be extendable. In some examples, the sterile sheath length may be retractable. In some examples, the sterile sheath length may be extended and retracted as the device enters the human body, so as to keep this section of the catheter in a sterile state for a long time and avoid infection as much as possible.

In some examples, the sterile sheath may be installed between the locking mechanism of the outer balloon catheter and the first hub of the inner balloon catheter. In some examples, the sterile sheath length may be expanded or contracted as the relative positions of the inner balloon catheter and the outer balloon catheter change, thereby keeping this section of the catheter in a sterile state for a longer period of time and avoiding infection as much as possible.

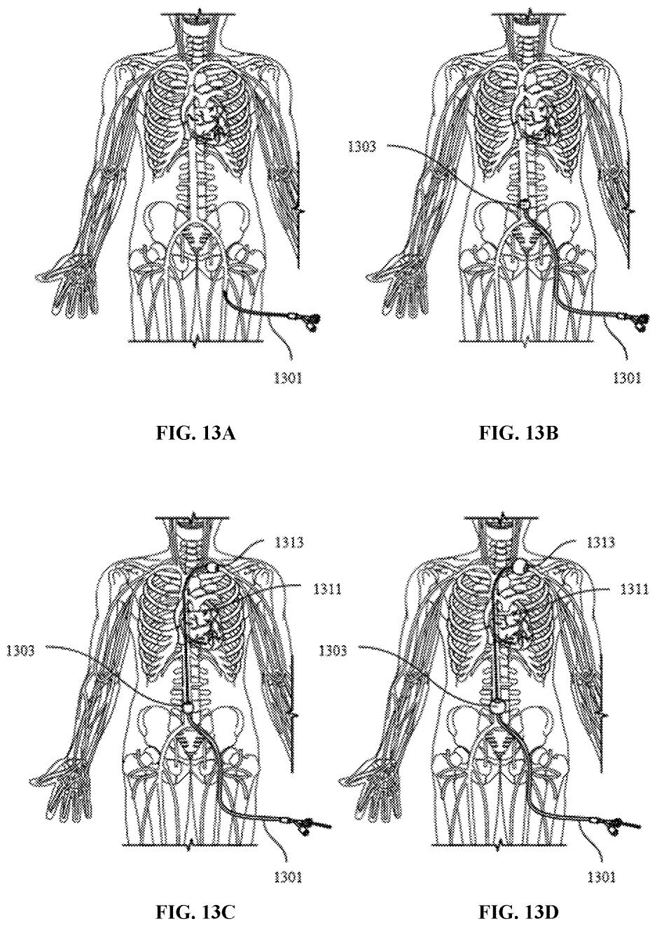

FIGS. 13A-13D are illustrations depicting a method for using the balloon catheter systems described herein to treat heart failure, according to some embodiments. FIG. 13A shows the step of introducing the balloon catheter system into the femoral vein through a puncture site. FIG. 13B shows the step of advancing the first balloon 1303 of the first balloon catheter 1301 to the first target location at the inferior vena cava (“IVC”) proximal to either the left or right renal veins. FIG. 13C shows the step of advancing the second balloon 1313 of the second balloon catheter 1311 to the second target location proximal to either the left or right subclavian vein. FIG. 13D shows the step of inflating the first balloon 1303 and second balloon 1313 of the balloon catheter system.

In some examples, the method may further comprises enlarging the puncture site using the detachable sheath and advancing the first balloon of the first balloon catheter through the detachable sheath to the first target location. In some examples, after the first balloon is positioned at the first target location, the detachable sheath may be removed, such as, for example, by tearing the detachable sheath using the first detachable arm and a second detachable arm.

In some examples, the first balloon may be advanced to the first target location using a guidewire. In some examples, the second balloon may be advanced to the second target location using a guidewire (“GW”).

In some examples, the device may comprise a radio marker. In some examples, the radio marker may allow for visualizing the catheter with aid of at least one radio marker of the catheter. In some examples, the first balloon may further comprise at least one radio marker allowing a user to visualize the first balloon under radiography or fluoroscopy, such that the user may accurately guide the first balloon to the first target location. In some examples, the second balloon may further comprise at least one radio marker allowing a user to visualize the second balloon under radiography or fluoroscopy, such that the user may accurately guide the second balloon to the second target location.

In some examples, intermittent occlusion of one or more veins may reduce venous blood backflow to the heart and/or reduces the pumping burden for the heart. In some embodiments, inflation of the first balloon may create a low-pressure area at or below the renal vein(s), improving blood and lymphatic return and organ function. For example, blood pressure at or below the renal vein(s), as measured by the first balloon catheter, may be reduced by about 5%, 10%, 15%, 20%, 25%, 30%, 35%, 40%, 45%, 50%, 55%, 60%, 65%, 70%, 75%, 80%, 85%, 90%, 95%, or any range therebetween with the first balloon inflated. In some embodiments, the low-pressure area in the renal vein may promote renal circulation, speed up urination, and/or reduce fluid retention in the body. In some examples, inflation of the first balloon may stimulate the vagus nerve causing enhanced blood accommodation in the lower extremities and abdomen. In some examples, stimulation of the vagus nerve can reduce venous blood backflow to the heart and reduce the pumping burden for the heart.

In some examples, the first balloon of the first balloon catheter may be positioned at a first target location in the inferior vena cava (“IVC”) proximal to either the left or right renal veins. In some examples, the first balloon may be positioned away from the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some examples, the first balloon may be positioned away from the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some examples, the first balloon may be from about 1 mm to about 25 mm away from the left or right renal vein. In some examples, the first balloon may be from about 5 mm to about 25 mm away from the left or right renal vein. In some examples, the first balloon may be from about 10 mm to about 25 mm away from the left or right renal vein. In some examples, the first balloon may be from about 15 mm to about 25 mm away from the left or right renal vein. In some examples, the first balloon may be from about 15 mm to about 20 mm away from the left or right renal vein.

In some examples, the first balloon may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some examples, the first balloon may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some examples, the first balloon may be inflated for a period from about 1 sec to about 60 sec. In some examples, the first balloon may be inflated for a period from about 10 sec to about 40 sec. In some examples, the first balloon may be inflated for a period from about 20 sec to about 40 sec. In some examples, inflation of the first balloon may occlude the inferior vena cava near the renal veins by about 10%, 20%, 30%, 40%, 50%, 60%, 70%, 80%, 90%, or any range therebetween.

In some examples, intermittent occlusion of one or more veins may reduce venous blood backflow to the heart and/or reduce the pumping burden for the heart. In some examples, inflation of the second balloon may create a low-pressure area near the left internal jugular vein and thoracic duct, improving return to the SVC and the right atrium of the heart, increases lymphatic reflow, and/or reduces fluid retention in the body. For example, blood pressure near the left internal jugular vein and thoracic duct, as measured by the second balloon catheter, may be reduced by about 5%, 10%, 15%, 20%, 25%, 30%, 35%, 40%, 45%, 50%, 55%, 60%, 65%, 70%, 75%, 80%, 85%, 90%, 95%, or any range therebetween with the second balloon inflated. In some examples, inflation of the second balloon may stimulate the vagus nerve, causing improved cardiac remodeling and function. In some examples, stimulation of the vagus nerve can cause vasodilation which may increase blood accommodation in the venous bed and reduces heart preload. In some examples, stimulation of the vagus nerve can reduce venous blood backflow to the heart and reduce the pumping burden for the heart.

In some examples, the second balloon may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some examples, the second balloon may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some examples, the second balloon may be inflated for a period from about 1 sec to about 60 sec. In some examples, the second balloon may be inflated for a period from about 10 sec to about 40 sec. In some examples, the first balloon may be inflated for a period from about 10 sec to about 30 sec. In some examples, inflation of the second balloon may occlude the subclavian vein by about 10%, 20%, 30%, 40%, 50%, 60%, 70%, 80%, 90%, or any range therebetween.

In some examples, the method may further comprise the step of measuring the pressure of at the subclavian vein using the second balloon catheter. In some examples, the measured pressure may be used to determine whether to inflate or deflate the first balloon and/or second balloon. In some examples, inflation or deflation of the first balloon may cause a pressure change (e.g., modulates the pressure) at or below the renal vein. For example, inflation of the first balloon may cause an increase in pressure below the renal vein. In some embodiments, when the pressure at the renal vein increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the first balloon is deflated. In some embodiments, when the pressure at the renal vein increases by about 1 mmHg to about 10 mmHg, the first balloon is deflated. In some embodiments, when the pressure at the renal vein increases by about 3 mmHg to about 5 mmHg, the first balloon is deflated.

Inflation or deflation of the second balloon may cause a pressure change (e.g., modulates the pressure) near the left internal jugular vein and thoracic duct. For example, inflation of the second balloon may cause an increase in pressure near the left internal jugular vein and thoracic duct. In some embodiments, when the pressure at the left internal jugular vein and thoracic duct increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the second balloon is deflated. In some embodiments, when the pressure at the left internal jugular vein and thoracic duct increases by about 1 mmHg to about 10 mmHg, the second balloon is deflated. In some embodiments, when the pressure at the left internal jugular vein and thoracic duct increases by about 5 mmHg to about 10 mmHg, the second balloon is deflated.

In some examples, the balloon may be advanced to the third target location using a guidewire. In some examples, the advanced balloon may be a third balloon. In some examples, the third balloon may be inserted by a catheter further into a lumen of the catheter system. In some examples, the third balloon of the catheter may be positioned at or near the pulmonary artery; In some examples, the third balloon may be inflated. In some examples, the pulmonary artery pressure of the subject may be measured with the catheter system.

In some examples, the outlet of the first balloon catheter may be positioned in the inferior vena cava below the left or right renal veins. In some examples, the outlet may be positioned below the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some examples, the outlet may be positioned below the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some examples, the outlet may be from about 1 mm to about 25 mm below the left or right renal vein. In some embodiments, the outlet may be from about 5 mm to about 20 mm below the left or right renal vein. In some examples, the outlet may be from about 5 mm to about 15 mm below the left or right renal vein. In some examples, the outlet is about 10 mm below the left or right renal vein.

In some examples, the balloon may comprise further at least one radio marker allowing a user to visualize the balloon under radiography or fluoroscopy, such that the user may accurately guide the balloon to the second target location.

In some examples, a method for insertion of a catheter device comprising multiple catheters may be disclosed. In some examples, the method comprises establishing vascular access via the femoral vein in a subject. In some examples, the method comprises inserting a catheter guidewire through the access. In some examples, the method comprises performing venography of the superior vena cava and inferior vena cava to determine the anatomical positions and courses. In some examples, the method may comprise measuring the diameter of the vessels at the intended balloon placement sites. In some examples, the method may comprise inserting the balloon catheters into corresponding position. In some examples, the method may comprise inflating the balloons to a desired volume. In some examples, the method may comprise injecting contrast medium through the vascular sheath. In some examples, the method may comprise observing under DSA to confirm balloon position. In some examples, the method may comprise observing under DSA to confirm the degree of vascular occlusion and determine inflation volumes. In some examples, the method may comprise inserting a Swan-Ganz catheter via the jugular vein to measure baseline and intro-treatment hemodynamic parameters. In some examples, the method may comprise connecting the balloon catheters to a pump or external system. In some examples, the method may comprise setting parameters for balloon occlusion and release. In some examples, the method may comprise performing automatic inflation and deflation of the balloon. In some examples, the method may comprise terminating after a set time period. In some examples, the method may comprise terminating after termination criteria have been met. In some examples, the method may comprise removal of catheters.

In some examples, the balloon may be positioned in the infrarenal IVC (inferior vena cava). In some examples, the balloon may be positioned along a vertebral level. In some examples, the balloon may be positioned between the L1 and L5 vertebrae. In some examples, the balloon may be positioned between the L1 and L2 vertebrae. In some examples, the balloon may be positioned in relation to the right renal vein. In some examples, the balloon may be positioned from about 5 mm to about 50 mm from the right renal vein.

In some examples, the balloon may be positioned in the left subclavian vein. In some examples, the balloon may be positioned along a vertebral level. In some examples, the balloon may be positioned between the C4 and T5 vertebrae. In some examples, the balloon may be positioned between the L7 and T1 vertebrae. In some examples, the balloon may be positioned in relation to the left internal jugular vein and the left subclavian vein. In some examples, the balloon may be positioned near the confluence of the left internal jugular vein and subclavian vein.

In some examples, the balloon may be inflated to a volume. In some examples, the balloon may be inflated to about 1% to about 100% of its maximum volume. In some examples, the balloon may be inflated to about 1% to about 20% of its maximum volume. In some examples, the balloon may be inflated to about 20% to about 40% of its maximum volume. In some examples, the balloon may be inflated to about 40% to about 60% of its maximum volume. In some examples, the balloon may be inflated to about 60% to about 80% of its maximum volume. In some examples, the balloon may be inflated to about 80% to about 100% of its maximum volume.

In some examples, the balloon may be inflated on a balloon inflation rate. In some examples, the balloon may be inflated from about 1 mL/sec (milliliter per second) to about 40 mL/sec. In some examples, the balloon may be inflated from about 1 mL/sec to about 10 mL/sec. In some examples, the balloon may be inflated from about 10 mL/sec to about 20 mL/sec. In some examples, the balloon may be inflated from about 20 mL/sec to about 30 mL/sec. In some examples, the balloon may be inflated from about 30 mL/sec to about 40 mL/sec. In some examples, the balloon inflation rate may be about 6 mL/sec.

In some examples, the balloon may be inflated to a volume. In some examples, the balloon volume may be from about 0.1 mL to about 20 mL. In some examples, the balloon volume may be from about 0.1 mL to about 5 mL. In some examples, the balloon volume may be from about 5 mL to about 10 mL. In some examples, the balloon volume may be from about 10 mL to about 15 mL. In some examples, the balloon volume may be from about 15 mL to about 20 mL. In some examples, the balloon volume may be about 0.3 mL. In some examples, the balloon volume may be about 5 mL. In some examples, the balloon volume may be about 6 mL.

In some examples, the occlusion parameters may comprise a vascular occlusion metric. In some examples, the balloon may be inflated to result in vascular occlusion. In some examples, the occlusion level may be from about 0% to about 100%. In some examples, the occlusion level may be from about 0% to about 20%. In some examples, the occlusion level may be from about 20% to about 40%. In some examples, the occlusion level may be from about 40% to about 60%. In some examples, the occlusion level may be from about 60% to about 80%. In some examples, the occlusion level may be from about 80% to about 100%. In some examples, the occlusion level may be about 70%, 85%, or 100%.

In some examples, the occlusion parameters may comprise an occlusion a portion of two or more veins. In some examples, the balloon may be inflated to result in occlusion of a portion of two or more veins. In some examples, the occlusion level may be from about 0% to about 100%. In some examples, the occlusion level may be from about 0% to about 20%. In some examples, the occlusion level may be from about 20% to about 40%. In some examples, the occlusion level may be from about 40% to about 60%. In some examples, the occlusion level may be from about 60% to about 80%. In some examples, the occlusion level may be from about 80% to about 100%. In some examples, the occlusion level may be about 70%, 85%, or 100%.

In some examples, intermittent occlusion of two or more veins may have effects. In some examples, intermittent occlusion of two or more veins may minimize hemodynamic disturbance. In some examples, minimization of hemodynamic disturbance may be characterized by maintenance of stable blood pressure, superior vena cava pressure, or inferior vena cava pressure. In some examples, minimization of hemodynamic disturbance may be characterized by a variation in pressure. In some examples, the pressure variation may be less than 50 mmHg, 45 mmHg, 40 mmHg, 35 mmHg, 30 mmHg, 25 mmHg, 20 mmHg, 15 mmHg, 10 mmHg, or 5 mmHg. In some examples, the pressure variation may be less than 20 mmHg In some examples, intermittent occlusion of two or more veins may achieve optimal therapeutic effects. In some examples, optimal therapeutic effect may be characterized by stabilization of hemodynamic disturbances. In some examples, intermittent occlusion of two or more veins may achieve optimal therapeutic effects for acute heart failure. In some examples, creation of a low-pressure area may reduce pulmonary artery pressure. In some examples, intermittent occlusion of two or more veins may demonstrate favorable acute and long-term safety. In some examples, favorable acute safety may be demonstrated by hemodynamic stability. In some examples, favorable long-term safety may be demonstrated by development of controllable vascular damage or thrombosis. In some examples, intermittent occlusion of the two or more veins may improve cardiac output. In some examples, improvement of cardiac output may be characterized by an overall mL/min increase over a period of time. In some examples, the period of time may be at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some examples, intermittent occlusion of the two or more veins may improve ventricular ejection fraction. In some examples, controllable vascular results may be found after a time period from treatment. In some examples, the time period from treatment may be from about 1 day to about 180 days. In some examples, the time period from treatment may be about 1 day, 7 days, 14 days, 30 days, 60 days, 90 days, 120 days, 150 days, or 180 days. In some examples, controllable vascular results may be characterized by controllable vascular damage or thrombosis.

In some examples, the method may terminate after a set time period, or a pre-determined time. In some examples, the pre-determined time may be about 1 minute to about 24 hours. In some examples, the pre-determined time may be about 1 minute to about 4 hours. In some examples, the pre-determined time may be about 4 hours to about 8 hours. In some examples, the pre-determined time may be about 8 hours to about 12 hours. In some examples, the pre-determined time may be about 12 hours to about 16 hours. In some examples, pre-determined time may be about 16 hours to about 20 hours. In some examples, the pre-determined time may be about 20 hours to about 24 hours.

In some examples, occlusion may be of the subclavian vein. In some examples, occlusion may be of the infrarenal verna cava. In some examples, vascular occlusion may be completed by a balloon, wherein the balloon may be the first or second balloon. In some examples, vascular occlusion may be from about 30 seconds to about 30 minutes. In some examples, vascular occlusion may be from about 30 seconds to about 5 minutes. In some examples, vascular occlusion may be from about 5 minutes to about 10 minutes. In some examples, vascular occlusion may be from about 10 minutes to about 15 minutes. In some examples, vascular occlusion may be from about 15 minutes to about 20 minutes. In some examples, vascular occlusion may be from about 20 minutes to about 25 minutes. In some examples, vascular occlusion may be from about 25 minutes to about 30 minutes.

In some examples, the method may comprise a synchronous and asynchronous inflation of the two or more balloons. In some examples, the two or more balloons may be inflated synchronously. In some examples, the two or more balloons may be inflated synchronously for 0% to 100% of the pre-determined time. In some examples, the two or more balloons may be inflated synchronously for 0% to 25% of the pre-determined time. In some examples, the two or more balloons may be inflated synchronously for 25% to 50% of the pre-determined time. In some examples, the two or more balloons may be inflated synchronously for 50% to 75% of the pre-determined time. In some examples, the two or more balloons may be inflated synchronously for 75% to 100% of the pre-determined time. In some examples, the two or more balloons may be inflated synchronously for 25%, 33%, or 66% of the pre-determined time. In some examples, the two or more balloons may be inflated asynchronously, where only one of the balloons are inflated. In some examples, the two or more balloons may be inflated asynchronously for 0% to 100% of the pre-determined time. In some examples, the two or more balloons may be inflated asynchronously for 0% to 25% of the pre-determined time. In some examples, the two or more balloons may be inflated asynchronously for 25% to 50% of the pre-determined time. In some examples, the two or more balloons may be inflated asynchronously for 50% to 75% of the pre-determined time. In some examples, the two or more balloons may be inflated asynchronously for 75% to 100% of the pre-determined time. In some examples, the balloon inflation may comprise maintenance of the balloon inflation before deflation. In some examples, inflating the first balloon may comprise maintenance of the first balloon inflation before deflation. In some examples, inflating the second balloon may comprise maintenance of the second balloon inflation before deflation. In some examples, inflation of the balloons may be adjusted with external input. In some examples, inflation of the balloons may be adjusted without external input. In some examples, inflation of the first balloon may be adjusted without external input. In some examples, inflation of the second balloon may be adjusted without external input. In some examples, inflation of the first and second balloons may be adjusted without external input.

In some examples, the method may comprise a cycle of occlusion of the IVC balloon and subclavian vein balloon. In some examples, the cycle may have a cycle time. In some examples, the cycle may be from about 1 minute to about 60 minutes. In some examples, the cycle may be from about 1 minute to about 20 minutes. In some examples, the cycle may be from about 20 minutes to about 40 minutes. In some examples, the cycle may be from about 40 minutes to about 60 minutes. In some examples, the cycle may about 6 minutes, 10 minutes, 11 minutes, 12 minutes, 13 minutes, 15 minutes, 20 minutes, 21 minutes, 40 minutes, or 50 minutes.

In some examples, the cycle may be repeated a number of times over a pre-determined time period. In some examples, the method pre-determined time may be about 1 minute to about 24 hours. In some examples, the pre-determined time may be about 1 minute to about 4 hours. In some examples, the pre-determined time may be about 4 hours to about 8 hours. In some examples, the pre-determined time may be about 8 hours to about 12 hours. In some examples, the pre-determined time may be about 12 hours to about 16 hours. In some examples, pre-determined time may be about 16 hours to about 20 hours. In some examples, the pre-determined time may be about 20 hours to about 24 hours.

In some examples, the cycle may comprise occlusion rate of a balloon, wherein the balloon may be the IVC balloon and/or the subclavian balloon. In some examples, the balloon may not be occluded. In some examples, the balloon occlusion may be from about 50% to about 100%. In some examples, the balloon occlusion may be from about 50% to about 75%. In some examples, the balloon occlusion may be from about 75% to about 100%. In some examples, the balloon occlusion may be about 70% or about 85%.

In some examples, the cycle may comprise a synchronous and asynchronous inflation of the two or more balloons. In some examples, the two or more balloons may be inflated synchronously. In some examples, the two or more balloons may be inflated synchronously for 0% to 100% of the pre-determined time. In some examples, the two or more balloons may be inflated synchronously for 0% to 25% of the pre-determined time. In some examples, the two or more balloons may be inflated synchronously for 25% to 50% of the pre-determined time. In some examples, the two or more balloons may be inflated synchronously for 50% to 75% of the pre-determined time. In some examples, the two or more balloons may be inflated synchronously for 75% to 100% of the pre-determined time. In some examples, the two or more balloons may be inflated synchronously for 25%, 33%, or 66% of the pre-determined time. In some examples, the two or more balloons may be inflated asynchronously, where only one of the balloons are inflated. In some examples, the two or more balloons may be inflated asynchronously for 0% to 100% of the pre-determined time. In some examples, the two or more balloons may be inflated asynchronously for 0% to 25% of the pre-determined time. In some examples, the two or more balloons may be inflated asynchronously for 25% to 50% of the pre-determined time. In some examples, the two or more balloons may be inflated asynchronously for 50% to 75% of the pre-determined time. In some examples, the two or more balloons may be inflated asynchronously for 75% to 100% of the pre-determined time.

In some examples, isolated occlusion of the intra-renal IVC may increase distal IVC pressure. In some examples, distal IVC pressure may increase by from about 5 mmHG to about 50 mmHg. In some examples, distal IVC pressure may increase by from about 5 mmHG to about 20 mmHg. In some examples, distal IVC pressure may increase by from about 20 mmHG to about 30 mmHg. In some examples, distal IVC pressure may increase by from about 30 mmHG to about 40 mmHg. In some examples, distal IVC pressure may increase by from about 40 mmHG to about 50 mmHg. In some examples, distal IVC pressure may increase by about 31 mmHG, from about 12 mmHg to about 43 mmHg.

In some examples, isolated occlusion of subclavian vein may increase subclavian vein pressure. In some examples, subclavian vein pressure may increase by from about 1 mmHG to about 50 mmHg. In some examples, subclavian vein pressure may increase by from about 1 mmHG to about 20 mmHg. In some examples, subclavian vein pressure may increase by from about 20 mmHG to about 30 mmHg. In some examples, subclavian vein pressure may increase by from about 30 mmHG to about 40 mmHg. In some examples, subclavian vein pressure may increase by from about 40 mmHG to about 50 mmHg. In some examples, subclavian vein pressure may increase by about 7 mmHG, from about 7 mmHg to about 14 mmHg.

In some examples, synchronous occlusion of the IVC and subclavian vein may reduce pulmonary artery pressure. In some examples, the pulmonary artery pressure may be decreased from about 1 mmHg to about 30 mmHg. In some examples, the pulmonary artery pressure may be decreased from about 1 mmHg to about 15 mmHg. In some examples, the pulmonary artery pressure may be decreased from about 15 mmHg to about 30 mmHg. In some examples, the pulmonary artery pressure may be decreased from about 30/20 mmHg to about 14/7 mmHg.

In some examples, simultaneous occlusion of the infra-renal IVC and subclavian vein may decrease pulmonary artery pressure. In some examples, the pulmonary artery may have a baseline pressure prior to occlusion. In some examples, the baseline pressure may be from around 5 mmHg to around 50 mmHg. In some examples, the baseline pressure may be from around 5 mmHg to around 25 mmHg. In some examples, the baseline pressure may be from around 25 mmHg to around 50 mmHg. In some examples, the baseline pressure may be from around 15 mmHg to around 35 mmHg. In some examples, the pulmonary artery may have a plateau pressure established as the balloon inflates. In some examples, the plateau pressure may be from around 1 mmHg to around 50 mmHg. In some examples, the plateau pressure may be from around 1 mmHg to around 25 mmHg. In some examples, the plateau pressure may be from around 25 mmHg to around 50 mmHg. In some examples, the plateau pressure may be from around 5 mmHg to around 20 mmHg.

In some examples, simultaneous occlusion of the infra-renal IVC and subclavian vein may increase subclavian vein pressure. In some examples, the subclavian vein may have a baseline pressure prior to occlusion. In some examples, the baseline pressure may be from around 1 mmHg to around 50 mmHg. In some examples, the baseline pressure may be from around 1 mmHg to around 25 mmHg. In some examples, the baseline pressure may be from around 25 mmHg to around 50 mmHg. In some examples, the baseline pressure may be from around 5 mmHg to around 15 mmHg. In some examples, the subclavian vein may have a plateau pressure established as the balloon inflates. In some examples, the plateau pressure may be from around 1 mmHg to around 50 mmHg. In some examples, the plateau pressure may be from around 1 mmHg to around 25 mmHg. In some examples, the plateau pressure may be from around 25 mmHg to around 50 mmHg. In some examples, the plateau pressure may be from around 10 mmHg to around 25mmHg.

In some examples, intermittent partial occlusion of the subclavian vein and infrarenal inferior vena cava provides hemodynamic stability. In some examples, intermittent partial occlusion may be characterized by occlusion of the subclavian vein and/or inferior vena cava of about 50% to about 95%. In some examples, occlusion may be from about 50% to about 75%. In some examples, occlusion may be from about 75% to about 95%. In some examples, occlusion may be about 70% or about 85%. In some examples, intermittent partial occlusion may take place over a time period from about 1 minute to about 30 minutes. In some examples, intermittent partial occlusion may take place over a time period from about 1 minute to about 15 minutes. In some examples, intermittent partial occlusion may take place over a time period from about 15 minutes to about 30 minutes. In some examples, intermittent partial occlusion may take place over a time period of about 10 minutes or about 20 minutes. In some examples, the fluctuations may be around 5 mmHg to about 20 mmHg. In some examples, the fluctuations may be around 5 mmHg to about 10 mmHg. In some examples, the fluctuations may be around 10 mmHg to about 15 mmHg. In some examples, the fluctuations may be around 15 mmHg to about 20 mmHg. In some examples, the fluctuations may be around 7 mmHg or about 16 mmHg. In some examples, the fluctuations may be a percentage of the baseline pressure, wherein the pressure fluctuation may be less than around 20%. In some examples, the pressure fluctuations may be less than around 15%, less than around 10%, less than around 5%, or less than around 1%. In some examples, the pressure fluctuations may be around 7% or around 16%.

In some examples, the method may comprise releasing balloon pressure over time. In some examples, releasing balloon pressure may be from about 30 seconds to about 30 minutes. In some examples, releasing balloon pressure may be from about 30 seconds to about 5 minutes. In some examples, releasing balloon pressure may be from about 5 minutes to about 10 minutes. In some examples, releasing balloon pressure may be from about 10 minutes to about 15 minutes. In some examples, releasing balloon pressure may be from about 15 minutes to about 20 minutes. In some examples, releasing balloon pressure may be from about 20 minutes to about 25 minutes. In some examples, releasing balloon pressure may be from about 25 minutes to about 30 minutes.

In some examples, the method may comprise a termination criteria. In some examples, the termination criteria may comprise when radial artery systolic blood pressure decreased by more than 10% from baseline, both balloons were simultaneously deflated for 1 minute. In some examples, the termination criteria may comprise when subclavian vein systolic pressure increased by more than 30% from baseline, or inferior vena cava systolic pressure increased by more than 30% from baseline, the balloon at the corresponding site was deflated for 1 minute. In some examples, the termination criteria may comprise the failure to maintain test subject hemodynamics.

In some examples, the method may comprise collecting hemodynamic data. In some examples, the method may comprise measuring heart rate. In some examples, the method may comprise measuring arterial blood pressure. In some examples, the method may comprise measuring central venous pressure. In some examples, the method may comprise measuring pulmonary artery pressure. In some examples, the method may comprise measuring pulmonary capillary wedge pressure. In some examples, the method may comprise measuring cardiac output. In some examples, the method may comprise measuring subclavian vein pressure. In some examples, the method may comprise measuring inferior vena cava pressure. In some examples, the method may comprise measuring left ventricular ejection fraction. In some examples, the method may comprise measuring left arterial end-diastolic volume.

In some examples, the method may comprise collecting hemodynamic data over a time period. In some examples, the hemodynamic data may be measured over a time interval from about 1 minute to about 30 minutes. In some examples, the hemodynamic data may be measured every 1 minute, 5 minutes, 10 minutes, 15 minutes, or any suitable interval.

In some examples, the tests could automatically control balloon inflation and release according to preset programming. In some examples, continuous balloon inflation, rather than intermittent occlusion, may cause vascular intimal injury. In some examples, prolonged continuous balloon inflation, rather than intermittent occlusion, can lead to thrombosis.

In some examples, incomplete occlusion can reduce the risk of thrombosis. In some examples, intermittent occlusion may improve cardiac output. In some examples, intermittent occlusion may improve ventricular ejection fraction. In some examples, intermittent occlusion may allow for controllable vascular results. In some examples, intermittent occlusion may allow for controllable vascular results after treatment. In some examples, intermittent occlusion may allow for controllable vascular results at least one day after treatment. In some examples, intermittent occlusion may allow for controllable vascular results at least seven days after treatment. In some examples, intermittent occlusion may allow for controllable vascular results at least 30 days after treatment. In some examples, intermittent occlusion may allow for controllable vascular results at least 60 days after treatment. In some examples, intermittent occlusion may allow for controllable vascular results at least 90 days after treatment.

In some examples, occlusion treatment may produce results. In some examples, occlusion treatment may result in hemodynamic results. In some examples, occlusion treatment may result in stable maintenance of vital signs during treatment. In some examples, occlusion treatment may result in relatively stable maintenance of heart rate during treatment. In some examples, occlusion treatment may result in relatively stable maintenance of blood pressure during treatment. In some examples, occlusion treatment may result in improved cardiac function. In some examples, occlusion treatment may result in decreased Pulmonary Capillary Wedge Pressure (PCWP) during treatment. In some examples, occlusion treatment may result in increased cardiac output during treatment. In some examples, occlusion treatment may result in a shortened left atrial diameter. In some examples, occlusion treatment may result in improved left ventricular ejection fraction. In some examples, occlusion treatment may result in expectable and controllable long-term vascular injury. In some examples, occlusion treatment may result in post-treatment successful resuscitation. In some examples, occlusion treatment may result in healthy subjects at least 30 days post-treatment. In some examples, occlusion treatment may result in endothelial vascular injury consistent with pathological changes associated with post-vascular injury repair with noticeable signs of recovery after treatment. In some examples, occlusion treatment may result in thrombosis consistent with the pathological changes associated with post-vascular injury repair with noticeable signs of recovery after treatment.

In some examples, incomplete occlusion of two or more veins can reduce the risk of thrombosis. In some examples, intermittent occlusion of two or more veins may improve cardiac output. In some examples, intermittent occlusion of two or more veins may improve ventricular ejection fraction. In some examples, intermittent occlusion of two or more veins may allow for controllable vascular results. In some examples, intermittent occlusion of two or more veins may allow for controllable vascular results after treatment. In some examples, intermittent occlusion of two or more veins may allow for controllable vascular results at least one day after treatment. In some examples, intermittent occlusion of two or more veins may allow for controllable vascular results at least seven days after treatment. In some examples, intermittent occlusion of two or more veins may allow for controllable vascular results at least 30 days after treatment. In some examples, intermittent occlusion of two or more veins may allow for controllable vascular results at least 60 days after treatment. In some examples, intermittent occlusion of two or more veins may allow for controllable vascular results at least 90 days after treatment. In some examples, histopathological assessments may be completed. In some examples, immediate histopathological assessments may be completed. In some examples, long-term histopathological assessments may be completed. In some examples, histopathological assessments may be completed to check for injury. In some examples, histopathological assessments may be completed to check for treatment site injury. In some examples, histopathological assessments may be completed to check for organ injury. In some examples, histopathological assessments may be completed to check for down-flow target organ injury.

In some examples, the devices and systems disclosed herein may be used in treatment of heart failure. In some examples, the devices and systems disclosed herein may be used in treatment of chronic heart failure. In some examples, the devices and systems disclosed herein may be used in treatment of acute heart failure. In some examples, the devices and systems disclosed herein may be used in treatment of acute decompensated heart failure (ADHF).

In some examples, the device and systems disclosed herein may be used to treat a human. In some examples, the human may have heart failure. In some examples, the human may have acute heart failure. In some examples, the human may have chronic heart failure. In some examples, the human may have acute decompensated heart failure. In some examples, the human may be Class I, II, III, or IV in the New York Heart Association (NYHA) Heart Failure Classification. In some examples, the human may have had anti-ADHF treatment in the last 48 hours. In some examples, the human may have had consistent fluid overload. In some examples, the human may have had pulmonary artery wedge pressure of more than 15 mmHg. In some examples, the human may have high NT-proBNP levels. In some examples, the human may be subjected to timeline monitoring, such as screening, treatment, Post-Op observation, and Discharge. In some examples, Post-Op observation may be from about 1 day to 180 days from operation. In some examples, monitoring may comprise demography, echocardiogram, NT-proBNP test, medication, treatment, right heart catheterization (RHC), intra-aortic balloon pumping (IBP), adverse effect or serious adverse effect monitoring, blood tests, tests for thrombosis, tests for vessel injury, vital sign testing, instances of rehospitalization, or instance of death. In some examples, adverse effects may be characterized as injection site effects. In some examples, adverse effects may be hematoma of the arm. In some examples, the human may have experienced improvement of cardiac preload after treatment. In some examples, the human may have experienced improvement of pulmonary circulation after treatment. In some examples, improvement may be signified by reduction of heart pressure, heart rate, right atrial pressure (RAP), pulmonary artery wedge pressure (PAWP), pulmonary artery pressure (PAP), or right ventricular pressure (RVP).

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Exemplary Two-Lateral Branch Catheter Instruments

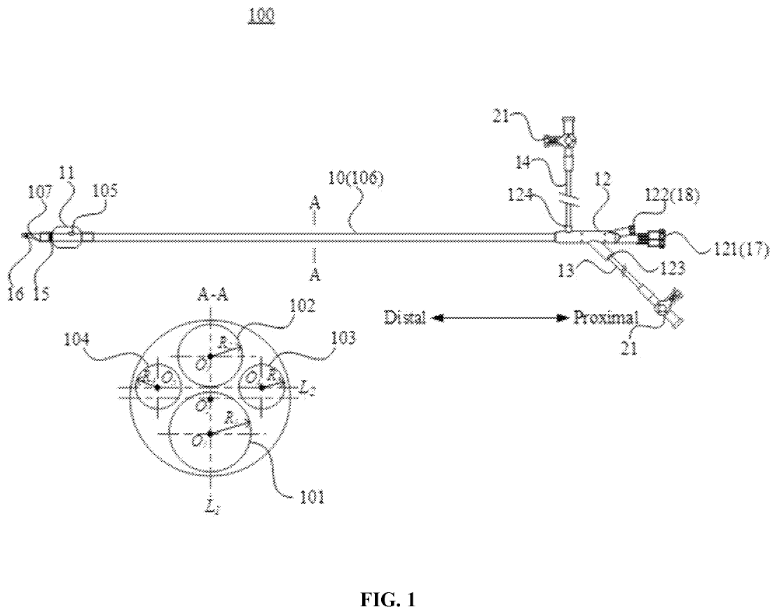

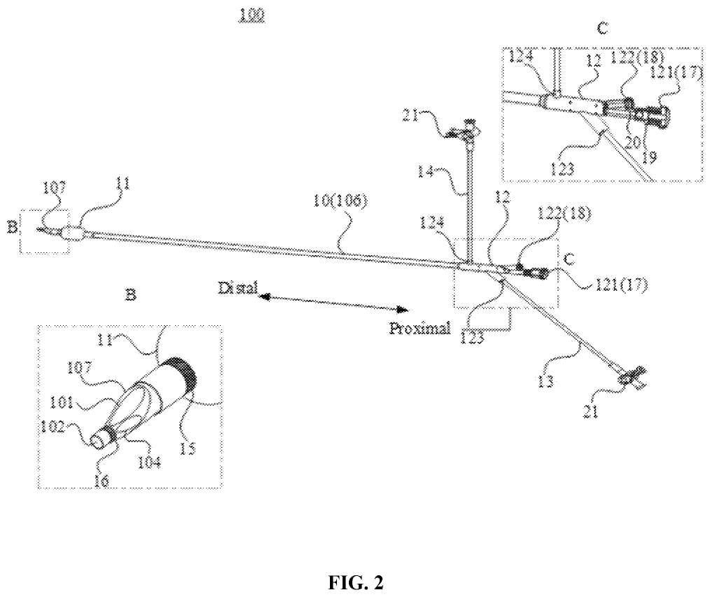

As shown in FIGS. 1 and 2, the catheterization device 100 includes a catheter body 10, a balloon 11, an attachment, or coupling, seat 12, a first lateral branch 13, and a second lateral branch 14. This catheter body 10 has a device pass-through lumen 101, a balloon catheter pass-through lumen 102, a balloon-filled lumen 103, and a pressure-monitoring lumen 104, all running co-planar and out of communication with one another. The balloon 11 is sealed to the outer wall of the catheter body 10 and connected to the distal end of the balloon inflation lumen 103. The coupling seat 12 is connected to the proximal end of the catheter body 10 and includes a port 121 communicating with the instrument pass-through lumen 101, a first side port 122 communicating with the balloon catheter pass-through lumen 102, a second side port 123 communicating with the balloon pressure-filling lumen 103, and a third side port 124 communicating with the pressure-monitoring lumen 104, wherein the port 121 is used for penetration of a diagnostic instrument (not shown), the first side port 122 is used for penetration of an instrument, and the first side port 122 is used for penetration of an instrument or a balloon catheter (not shown). The first side branch 13 is in communication with the second side port 123 and is used for inputting a balloon inflation medium, and the second side branch 14 is in communication with the third side port 124 and is used for inputting a pressure monitoring medium.

The catheterization instrument 100 of the presently disclosed embodiment is used to provide access for balloon catheters as well as other diagnostic instruments, which can be used to seal the blood vessel by filling the balloon 11 with the aid of an external pressure pump, and can also be used to monitor the intravascular pressure by means of an external pressure sensor, utilizing the pressure-monitoring lumen 104, during the procedure. The diagnostic instrument can be threaded into the instrument passage lumen 101 from the port 121 of the connection seat 12, and the balloon catheter can be threaded into the balloon catheter passage lumen 102 from the first side port 122 of the connection seat 12, and the balloon filling lumen 103 can be filled with pressure-filling medium with the aid of an external device utilizing the first side branch 13, and the pressure-monitoring medium can be filled with pressure-monitoring medium into the pressure-monitoring lumen 104 utilizing the second side branch 14. A luer fitting tee 21 is provided at the proximal end of each of the first side branch tube 13 and the second side branch tube 14, thereby allowing a plurality of mutually compatible fluids to be managed using the same line.

The diagnostic and therapeutic instrument may be an inspection catheter, a floating catheter, an ablation catheter, or any similar catheter. The pressure-monitoring medium may be, for example, heparin saline, and the present disclosure does not make specific limitations thereon, and the selection may be made according to the actual treatment plan.

After the catheterization device 100 is placed in the body, a balloon catheter and/or a diagnostic device may be placed, guided by the catheterization device 100 and associated guide member, so that the blood vessel may be occluded using the balloon 11 of the catheterization device 100 and the balloon of the balloon catheter, and the diagnostic device may be used to carry out the associated diagnostic operation to monitor intravascular pressure by means of pressure monitoring of the lumen 104, which is directed to the distal end of the catheterization device 100 and leads into the blood vessel. The intravascular pressure is monitored. A process of placing the catheterization device 100 and the balloon catheter in the human body is in the following order: performing a puncture using a puncture needle→placing a short guidewire in the puncture needle →withdrawing the puncture needle→placing the catheterization device 100 using the short guidewire as a guide→withdrawing the short guidewire→placing a long guidewire into the balloon catheter of the catheterization device 100 through the lumen 102→placing the balloon catheter using the long guidewire as a guide→withdrawing the long guidewire. In this regard, the short guidewire and the long guidewire are used as intermediate auxiliary instruments when placing the catheterization instrument 100 and the balloon catheter.

According to the design of embodiments of the present disclosure, the balloon 11 of the catheterization device 100 and the balloon intermittently passing through the balloon catheter of the catheterization device 100 may be utilized to seal the veins, thereby reducing the return of venous blood to the heart, reducing the preload on the heart, and allowing for a reduction in ventricular wall stress. During or before and after treatment, the instrument can be passed through the lumen 101 for diagnostic and therapeutic instruments for relevant testing and treatment, and the intravascular pressure can be monitored through the pressure monitoring lumen 104. The catheterization device 100 of the presently disclosed embodiments can be used to treat acute congestive heart failure and can achieve good prognostic results.

The cross-sectional shape and arrangement of each lumen of the catheter body 10 is not limited. As shown in FIG. 1, the instrument pass-through lumen 101, the balloon catheter pass-through lumen 102, the balloon pressure-filling lumen 103, and the pressure-monitoring lumen 104 are each in the shape of a circular hole, wherein the center of a circle O1 of the cross-section of the instrument pass-through lumen 101, the center of a circle O2 of the cross-section of the catheter body 10, and the center of a circle O3 are disposed sequentially along the first linear direction L1, and the center of circle O4 of the cross-section of the balloon pressure-filling lumen 103 and the center of circle O5 of the cross-section of the pressure-monitoring lumen 104 are disposed sequentially along the second linear direction L2 orthogonal to the first linear direction L1.

Since the instrument passes through the lumen 101 to be passed into the diagnostic and therapeutic instrument, the aperture thereof is designed to be relatively large in order to enable it to pass through smoothly. The aperture diameter of the balloon catheter passage lumen 102 may be designed accordingly to the outer diameter of the balloon of the balloon catheter in the contracted state, so as to enable smooth passage of the balloon catheter, and the balloon inflation lumen 103 and the pressure monitoring lumen 104 may be disposed on both sides of the instrument passage lumen 101 and the balloon catheter passage lumen 102. The aperture R₁ of the device passage lumen 101, the aperture R₂ of the balloon catheter passage lumen 102, the aperture R₃ of the balloon-filled lumen 103, and the aperture R₄ of the pressure-monitoring lumen 104, satisfy the following: R₁>R(₂)>R₃=R₄.

As shown in FIG. 1, the catheter body 10 is provided with at least one through-hole 105 (e.g., a hole may be made directly in the outer wall of the catheter body 10 to form the at least one through-hole 105) connecting the balloon pressure-filled lumen 103 to the balloon 11, and the balloon 11 is a compliant tubular-wall shaped balloon that is in a constricted state against the outer wall of the catheter body 10.

The proximal inner wall and distal inner wall of the compliant wall-like balloon may be seal bonded to the outer wall of the catheter body 10, thereby sealing the balloon 11.

The balloon 11 is made of a compliant material, which may include, for example, at least one of silicone, latex, polyurethane, and polyimide ester, such that a larger expansion diameter can be obtained at lower pressures, and the expansion diameter grows significantly as the pressure is elevated, so that it can be effectively anchored after expansion to provide reliable support in the blood vessel. In addition, the balloon 11 adopts a wall-like design, which fits the outer wall of the catheter body 10 in the contracted state, so that a smaller outer diameter can be obtained, in order to minimize the resistance to travel in the blood vessel and reduce the harm to the human body.

As shown in FIG. 2, the catheter body 10 includes a main body portion 106 and a tip portion 107 connected to a distal end of the main body portion 106, wherein the instrumentation passes through a distal end of the lumen 101 and the pressure monitoring lumen 104 to the side of the tip portion 107, and the balloon catheter passes through a distal end of the lumen 102 to the end face of the tip portion 107. That is, the diagnostic instrument may pass through the side of the tip portion 107 of the catheter body 10 and the balloon catheter may pass through the end face of the tip portion 107 of the catheter body 10.

The material of the body portion 106 may, for example, comprise at least one of a polyamide, a polyether block polyamide, an acrylonitrile-butadiene-styrene terpolymer, and a polyethylene glycol p-toluene dicarboxylate. The tip portion 107 may be made of a material that is more flexible compared to the body portion 106, and may for example comprise at least one of polyurethane, polyimide ester, polyether block polyamide, and silicone. The tip portion 107 may be welded together with the body portion 106. The choice of material and shape of the tip portion 107 can be designed to reduce the resistance of the catheterization device 100 to traveling through the blood vessel, allowing for a smoother entry

Referring to FIG. 1, the catheterization instrument 100 may further include a first developing element 15 disposed on the outer wall of the main body portion 106, and/or a second developing element 16 disposed on the side of the tip portion 107. During surgery, the developing element may be detected using an external image detecting device (e.g., an X-ray detecting device), so as to accurately know its position, and to achieve precise positioning for instrumental Precise positioning of the intervention can be achieved. The material of the above-described developing element may be selected from one or more of gold, platinum, iridium, tantalum, tungsten and the like.

In embodiments of the present disclosure, the material of the connection seat 12 may include at least one of acrylonitrile-butadiene-styrene terpolymer, polyamide, polycarbonate, and polyformaldehyde. The connection seat 12 may provide access to other instruments or therapeutic media and is made of a material that is stiff relative to the catheter body 10 so as to facilitate assembly and instrument manipulation. In addition, a hemostatic valve may be installed therein.

As shown in FIG. 2, the catheter instrument 100 further includes: a first gland 17 disposed in the port 121, and a first hemostatic valve 19 disposed within the port 121 and secured by the first gland 17, a second gland 18 disposed in the first side port 122, and a second hemostatic valve 20 disposed within the first side port 122 and secured by the second gland 18. The hemostatic valve can use its own elasticity to squeeze and seal, preventing blood from flowing out and realizing a hemostatic effect. The material of the first hemostatic valve 19 and the second hemostatic valve 20 may include, for example, at least one of silicone, latex, and polyurethane.

The first hemostatic valve 19 is a radial compression hemostatic valve with an axial opening, and the first gland 17 cooperates with its periphery, so that when the first gland 17 is screwed down, it presses on the periphery of the first hemostatic valve 19, causing it to contract to close the hole, thereby providing hemostasis.

The second hemostatic valve 20 is a cross-cut hemostatic valve, which is in the form of a sheet and has a cross-cut, and in the natural state, the cross-cut is in a closed state, and when the balloon catheter passes through the cross-cut, it will open the cross-cut, but because the hemostatic valve itself has elasticity, it can be squeezed and wrapped around the surface of the balloon catheter, and provide a sealing and hemostatic effect.

Embodiments of the present disclosure also provide a heart failure treatment device comprising the catheterization device 100 of any of the preceding embodiments.

The heart failure treatment device may include associated diagnostic devices, balloon catheters, pumps, and pressure transducers. The heart failure therapeutic device may be used in a venous occlusion regimen, allowing for a favorable prognosis for the treatment of acute congestive heart failure.

Examples of therapeutic implementations of heart failure treatment devices according to some embodiments of the present disclosure are as follows:

First, a channel is established in the femoral vein by means of a puncture kit and a guidewire is placed; then, the catheterization device 100 is introduced into the body along the guidewire from the femoral vein, and the balloon 11 of the catheterization device 100 is positioned under contrast to the inferior vena cava, which is positioned underneath the renal vein, to secure the catheterization device 100.

Then, a guidewire is placed in the balloon catheter through the lumen 102 of the catheterization apparatus 100, so that the guidewire passes upwardly along the inferior vena cava through the heart, the superior vena cava, and the vein of the head and arm to the subclavian vein; then, the balloon catheter is placed to reach the designated position along the guidewire through the balloon catheter through the lumen 102, and in the process, the contrast agent may be delivered to the blood vessel through the contrast agent delivery holes that are provided with the balloon catheter, so as to enable the balloon catheter's balloon to accurately reach the blocking location.

After that, the catheterization instrument 100 and the balloon catheter are each connected to a pressure pump to start the treatment process.

During or before or after treatment, the instrument can be passed through the lumen 101 to a diagnostic device for relevant testing and treatment, the diagnostic device can be, for example, a floating catheter, an ablation catheter, etc., and in addition, the intravascular pressure can be monitored by the pressure-monitoring lumen 104 during the treatment.

It can be seen that for venous occlusion, the treatment plan can be to occlude the veins by two balloons working together, thus reducing the venous return to the heart and reducing the cardiac preload.

### Example 2: Exemplary Catheter Instruments With Five Lumen's

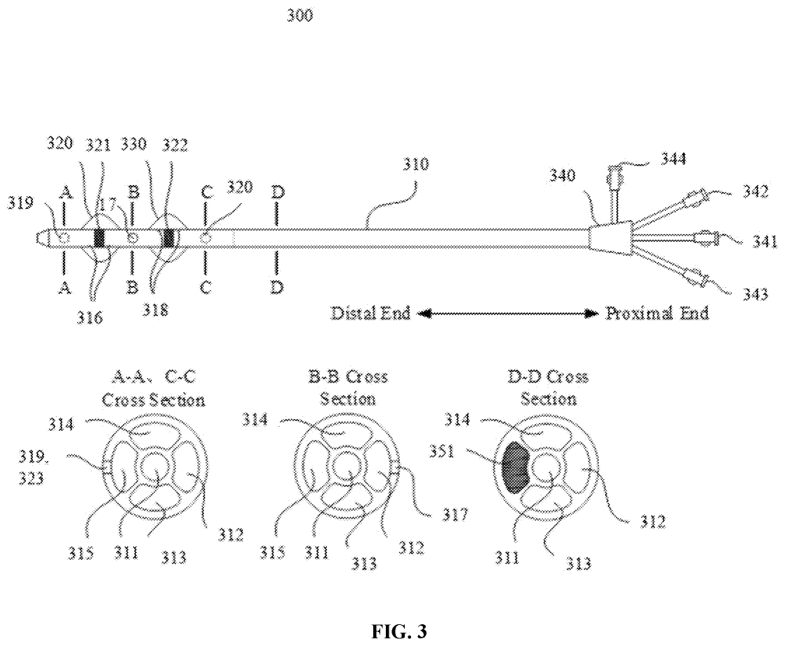

In FIG. 3, a catheter device 300 provided in some embodiments of the present disclosure includes a multi-lumen tube 310, a first balloon 320, and a second balloon 330. The multi-lumen tube 310 has a first lumen 311, a second lumen 312, a third lumen 313 and a fourth lumen 314 which are not connected to each other, and the wall of the multi-lumen tube 310 is provided with a first through-hole 316, a second through-hole 317 and a third through-hole 318 in sequence in a direction away from the distal end wherein the first lumen 311 runs through the multi-lumen tube 310 and is used for a guide wire (not shown in the figure) (i.e., a long guide wire hereinafter) to penetrate, the second lumen 312 is communicated with the second through-hole 317 and is used to transport lymph or contrast fluid through the second through-hole 317, the third lumen 313 is communicated with the first through-hole 316 and extends to the proximal end of the multi-lumen tube 310 and is used to transport a pressurized medium through the first through-hole 316, and the fourth lumen 314 is communicated with the third through-hole 318 and extends to the proximal end of the multi-lumen tube 310 and is used to transport a pressurized medium through the third through-hole 318. The first balloon 320 is sealedly connected to the outer wall of the multi-lumen tube 310 and is communicated with the third lumen 313 through the first through-hole 316. The second balloon 330 is sealed to the outer wall of the multi-lumen tube 310 and communicates with the fourth lumen 314 through the third through-hole 318. In this embodiment, the first lumen 311 runs through the multi-lumen tube 310, and the distal ends of the second lumen 312, the third lumen 313 and the fourth lumen 314 are all in a closed state.

The catheter device 300 of the embodiment of the present disclosure is used to transport contrast fluid to the lymphatic vessels or extract lymph fluid from the lymphatic vessels.

The operation process of inserting the above-mentioned catheter device 300 into the human body is as follows: use a puncture needle to perform puncture [symbol] insert a short guide wire into the puncture needle [symbol] pull out the puncture needle [symbol] insert the sheath tube with the short guide wire as a guide [symbol] pull out the short guide wire [symbol] insert a long guide wire with the sheath tube as a guide [symbol] insert the above-mentioned catheter device 300 provided in the embodiment of the present disclosure with the long guide wire as a guide (the long guide wire passes through the first lumen 311) and position it (with the help of external imaging detection equipment) [symbol] pull out the long guide wire.

After the catheter device 300 is placed in the human body and positioned, firstly, the pressurized medium is filled into the first balloon 320 through the third lumen 313 and into the second balloon 330 through the fourth lumen 314 by using an external pressurized medium input device, so that the first balloon 320 and the second balloon 330 can block the blood vessel segment containing the lymphatic duct opening in the filled state, and at this time, a negative pressure area can be created near the lymphatic duct opening; then, the contrast fluid can be input into the lymphatic vessel through the second lumen 312, the second through-hole 317 and the valve structure of the lymphatic duct opening in sequence by using an external contrast fluid input device, or, by using a suction device, the lymph fluid can be extracted out through the valve structure of the lymphatic duct opening, the second through-hole 317 and the second lumen 312 in sequence under the action of negative pressure. The catheter device 300 of this embodiment does not need to enter the lymphatic vessel when intervening in the human body.

The use of the catheter device 300 of the embodiment of the present disclosure in the treatment of heart failure can enhance the absorption and drainage functions of the lymphatic system, thereby more efficiently transferring the stagnant interstitial fluid, effectively alleviating fluid retention, and improving the effect of heart failure treatment.

In FIG. 3, the distal end of the multi-lumen tube 310 may be designed to be tapered, so that it can move smoothly in the blood vessel.

As shown in FIG. 3, the catheter device 300 further includes: a first developing element 321 disposed on the periphery of the multi-lumen tube 310 and located in the first balloon 320, and a second developing element 322 disposed on the periphery of the multi-lumen tube 310 and located in the second balloon 330.

The first developing element 321 and the second developing element 322 may be annular developing elements, which are attached to the outer wall of the multi-lumen tube 310. During surgery, the first developing element 321 and the second developing element 322 may be detected by an external image detection device (such as an X-ray detection device), so as to accurately know their positions, achieve accurate occlusion of the blood vessel segment and accurate positioning of the lymphatic vessel orifice. The material of the first developing element 321 and the second developing element 322 may be selected from one or more materials such as gold, platinum, iridium, tantalum, and tungsten.

In FIG. 3, the multi-lumen tube 310 also has a fifth lumen 315 that is not connected to the first lumen 311, the second lumen 312, the third lumen 313 and the fourth lumen 314, and the tube wall of the multi-lumen tube 310 is also provided with a fourth through-hole 319 and a fifth through-hole 323, wherein the fourth through-hole 319, the first through-hole 316, the second through-hole 317, the third through-hole 318 and the fifth through-hole 323 are arranged in sequence along the direction away from the distal end of the multi-lumen tube 310, the distal and proximal ends of the fifth lumen 315 are closed, and the fifth lumen 315 is connected to the fourth through-hole 319 and the fifth through-hole 323, and is used to drain blood through the fourth through-hole 319 and the fifth through-hole 323.

In this embodiment, even if the first balloon 320 and the second balloon 330 block the blood vessel segment containing the lymphatic vessel opening in the filled state, blood can still be drained through the fourth through-hole 319, the fifth lumen 315 and the fifth through-hole 323, thereby achieving smooth flow. In this way, the surgical risks caused by blood vessel blockage can be greatly reduced or even avoided.

In FIG. 3, the catheter device 300 further includes a connection seat 340, which is connected to the proximal end of the multi-lumen tube 310 and has a first connector 341 connected to the first lumen 311, a second connector 342 connected to the second lumen 312, a third connector 343 connected to the third lumen 313, and a fourth connector 344 connected to the fourth lumen 314. The first connector 341 is used for the insertion of a guide wire, the second connector 342 is used for transporting lymph or contrast fluid, and the third connector 343 and the fourth connector 344 are used for transporting pressurized media. Some of the above connectors can be Luer connectors, thereby allowing multiple compatible fluids to be managed using the same pipeline.

In FIG. 3, lymph flows out of the catheter device 300 from the second connector 342 and can then enter the human body's venous vessels again through other catheters via extracorporeal circulation, thereby returning to the human body's circulation.

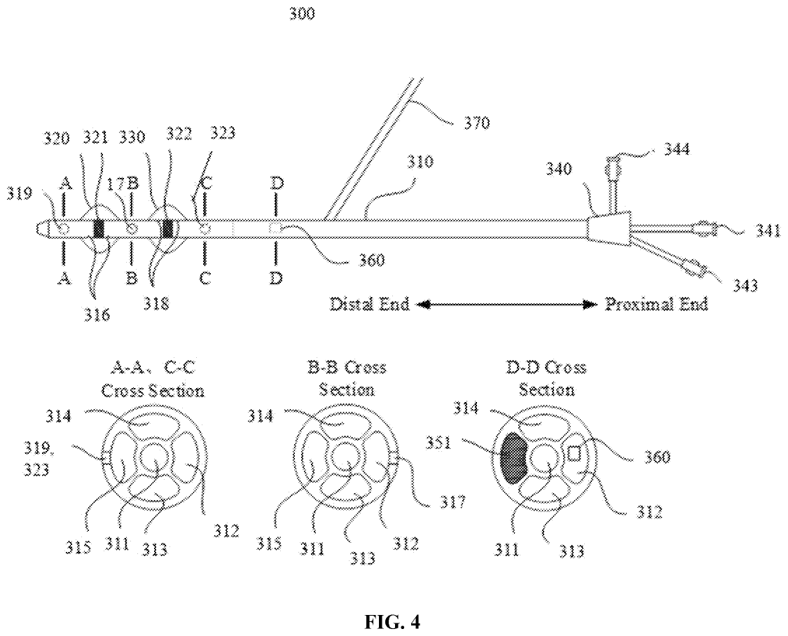

In FIG. 4, the distal end and the proximal end of the second lumen 312 of the catheter device 300 are both closed, and the catheter device 300 also includes an axial flow pump 360 disposed in the second lumen 312, and a return pipe 370 connected to the second lumen 312, and the return pipe 370 is used to transport the lymph fluid in the second lumen 312 to the venous blood vessel under the action of the axial flow pump 360. This embodiment utilizes the negative pressure generated by the axial flow pump 360 in the second lumen 312 to suck the lymph fluid into the second lumen 312, and then the lymph fluid is sent to the venous blood vessel through the return pipe 370 and the body circulation, and since the lymph fluid does not circulate extracorporeally, this can effectively reduce the risk of surgery.

In FIG. 3, the second lumen 312, the third lumen 313, the fourth lumen 314 and the fifth lumen 315 are distributed around the first lumen 311, and the third lumen 313 and the fourth lumen 314 are arranged opposite to each other. The cross-sectional shape of the first lumen 311 can be circular, and the second lumen 312, the third lumen 313, the fourth lumen 314 and the fifth lumen 315 can be designed with the same cross-sectional shape. The arrangement of the lumens in this embodiment can balance the pressure, which is conducive to improving the delivery efficiency and extending the service life.

The proximal end of the fifth lumen 315 is blocked with the proximal end of the multi-lumen tube 310 by a blocking material 351, and the distal end of the fifth lumen 315 is blocked with the distal end of the multi-lumen tube 310 by a blocking material 351, thereby forming a fifth lumen 315 with both ends closed. That is, when the multi-lumen tube 310 is manufactured, a conventional extrusion process is first used to form a five-lumen tube body, and then the blocking material 351 is filled to form the fifth lumen 315, so that the manufacturing process of the multi-lumen tube 310 is relatively simple. Similarly, the distal ends of the second lumen 312, the third lumen 313, and the fourth lumen 314 can also be closed by blocking materials.

In the embodiment of the present disclosure, the number of the first through-hole 316 and the number of the third through-hole 318 are respectively plural, so that the efficiency of charging the first balloon 320 and the second balloon 330 can be improved to make them expand quickly.

In the disclosed embodiment, the first balloon 320 and the second balloon 330 are made of compliant materials, which can obtain a larger expansion diameter at a lower pressure, and as the pressure increases, the expansion diameter increases significantly, so that after expansion, they can be effectively anchored and provide reliable support in the blood vessel. The materials of the first balloon 320 and the second balloon 330 can respectively include at least one of polyamide, polyurethane, medical latex, medical silicone, polyamide polyether block copolymer, and polyethylene.

The present disclosure also provides a heart failure treatment device, including the catheter device 300 of any of the above embodiments. In addition to the catheter device 300, the heart failure treatment device can also be equipped with a contrast fluid input device, a lymphatic fluid suction device, a pressurized medium input device, an X-ray detection device, etc.

Based on the above-mentioned design of the catheter device 300, a heart failure treatment device can be used to obtain a better heart failure treatment effect.

### Example 3: Exemplary Balloon Sheath

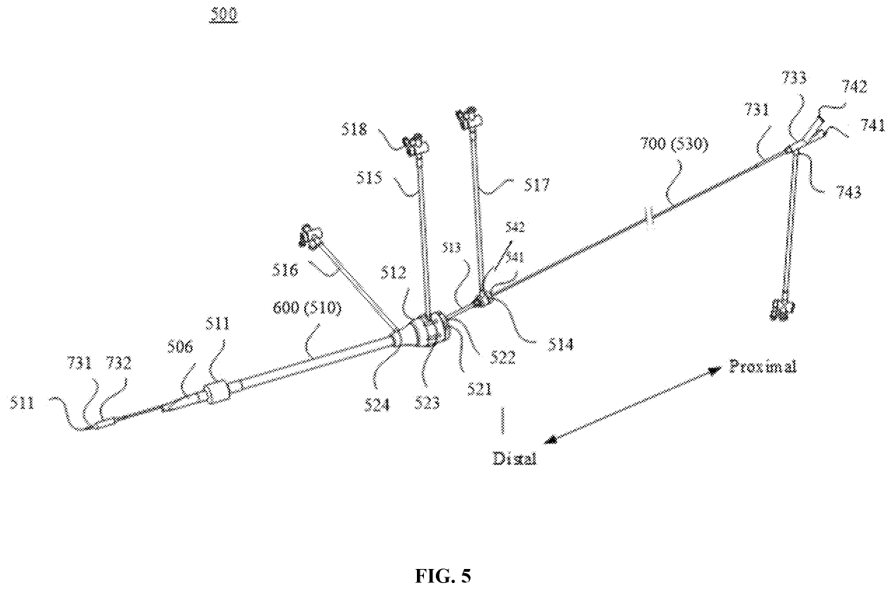

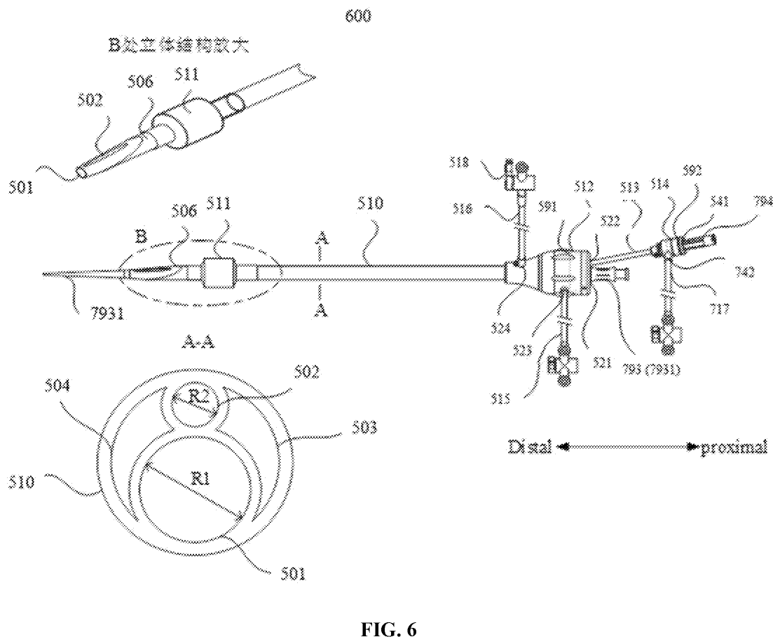

As shown in FIGS. 5 and 6, some embodiments of the present disclosure provide a balloon sheath 600 applied to a heart failure treatment device 500. The balloon sheath 600 comprises a main sheath 510, a first balloon 511, a main connection seat 512, a secondary sheath 513, a secondary connection seat 514, and three lateral branches. The main sheath 510 has a main lumen 501, a secondary lumen 502, a first pressure-filled lumen 503, and a second pressure-filled lumen 504 that are not in communication with each other, wherein the main lumen 501 is used to guide a diagnostic and treatment instrument (not shown in the drawings) through, and the secondary lumen 502 is used to guide the balloon catheter 700 through. The first balloon 511 is sealingly connected to the outer wall of the main sheath 510 and is connected to the distal ends of the first pressurized lumen 503 and the second pressurized lumen 504. The main connector 512 is connected to the proximal end of the main sheath 510 and includes a first port 521 in communication with the main lumen 501, a second port 522 in communication with the secondary lumen 502, a first side port 523 in communication with the main lumen 501, and second side ports 524 in communication with the first pressurized lumen 503 and the second pressurized lumen 504, wherein the first port 521 is for threading of diagnostic and therapeutic instruments. The secondary sheath tube 513 is in communication with the second port 522. The secondary connector 514 is connected to the proximal end of the secondary sheath tube 513 and includes a third port 541 and a third side port 542 in communication with the secondary sheath tube 513, wherein the third port 541 is for threading of the balloon catheter 700. The first side branch tube 515 is in communication with the first side port 523, the second side branch tube 516 is used to input a pressure-filled medium and is in communication with the second side port 524, and the third side branch tube 517 is in communication with the third side port 542.

In embodiments of the present disclosure, the balloon sheath 600, for example, defines the end of the balloon sheath 600 or component thereof that extends closer to the operator in the direction of extension as the “proximal end”, and similarly defines the end of the balloon sheath 600 or component thereof that extends further away from the operator in the direction of extension as the “distal end”.

The balloon sheath 600 of embodiments of the present disclosure is used to provide access to the balloon catheter 700 and other diagnostic devices and to seal blood vessels by means of its balloon. The diagnostic instruments can be threaded into the main lumen 501 from the first port 521 of the main connector 512, the balloon catheter 700 can be threaded into the secondary lumen 502 from the third port 541 of the secondary connector 514, the main lumen 501 can be filled with a flushing fluid using the first side branch 515, the first pressurized lumen 503 and the second pressurized lumen 504 can be filled with pressurized medium using the second side branch 516, and the secondary lumen 502 can be filled with pressurized media using the third side branch 517, and the secondary lumen 502 can be filled with pressurized medium using the third side branch 517. to fill the secondary lumen 502 with flushing fluid or to draw blood. A luer fitting tee is provided at the proximal end of each of the first side branch 515, the second side branch 516, and the third side branch 517, thereby allowing multiple mutually compatible fluids to be administered using the same line.

Following placement of the balloon sheath 600 in the human body, a balloon catheter 700 and/or a diagnostic device may be placed using the balloon sheath 600 and the associated guide member as a guide, thereby allowing the vessel to be occluded using the first balloon 511 of the balloon sheath 600 and the second balloon 732 of the balloon catheter 700 to perform the associated diagnostic and therapeutic operation using the diagnostic device. The operation process of placing the balloon sheath tube 600 and the balloon catheter 700 in the human body is, in order: performing a puncture using a puncture needle→placing a short guidewire in the puncture needle →pulling out the puncture needle→placing the balloon sheath tube 600 guided by the short guidewire (the first port 521 of the balloon sheath tube 600 is pre-positioned with the main expander 793 (shown in FIG. 6)), where the single-lumen catheter 7931 extends out of the distal end of the balloon sheath 600, and the short guide wire is threaded through the main lumen 501 of the balloon sheath 600)→pulling out the short guide wire and the main dilator 793→threading the long guide wire through the secondary lumen 502 of the balloon sheath 600 through the secondary dilator 794 pre-positioned in the third port 541 of the secondary connector seat 514→pulling out the secondary dilator 794→placing the long guide wire guided into the balloon catheter 700→and pulling out the long guide wire. Therein, the short guide wire, the long guide wire, the main dilator 793, and the sub-dilator 794 serve as intermediate auxiliary instruments when placing the balloon sheath tube 600 and the balloon catheter 700.

According to the design of embodiments of the present disclosure, on the one hand, the first balloon 511 of the balloon sheath tube 600 and the second balloon 732 of the balloon catheter 700 passing through the balloon sheath tube 600 can be utilized to intermittently block the vein, thereby reducing the return of venous blood to the heart, lowering the preload of the heart, and making the ventricular wall stress lower; on the other hand, it is possible for the first balloon 511 of the balloon sheath tube 600 and the second balloon 732 of the balloon catheter 700 passing through the balloon sheath tube 600 to be used to block the vein. On the other hand, the first balloon 511 of the balloon sheath tube 600 and the second balloon 732 of the balloon catheter 700 passing through the balloon sheath tube 600 are synchronized to inflate and deflate in accordance with a cardiac rhythm, thereby generating a dual hemodynamic effect, elevating the diastolic blood pressure and coronary perfusion, and lowering the afterload of the heart to improve the ejection of the left ventricle. The main lumen 501 of the balloon sheath 600 may also be passed into the diagnostic device for relevant testing and treatment during or before and after the treatment. Thus, the balloon sheath tube 600 of the presently disclosed embodiment can be used for treating acute congestive heart failure and can obtain a good prognosis.

The cross-sectional shape and arrangement of each lumen of the main sheath tube 510 are not limited. As shown in FIG. 6, the main tubing lumen 501 and the secondary tubing lumen 502 are each in the shape of a circular hole, wherein the aperture Ri of the main tubing lumen 501 is larger than the aperture R₂ of the secondary tubing lumen 502, and the first pressure-filled tubing lumen 503 and the second pressure-filled tubing lumen 504 are located on both sides of the main tubing lumen 501 and the secondary tubing lumen 502. Since the main tube lumen 501 needs to be passed into the diagnostic and therapeutic instruments, the aperture thereof is designed to be relatively large in order to enable smooth passage thereof. The aperture of the secondary lumen 502 may be designed accordingly based on the outer diameter of the second balloon 732 of the balloon catheter 700 in a contracted state, so as to enable smooth passage of the balloon catheter 700. The main sheath 510 may be molded using an extrusion process, with the lumen spaces on either side of the main lumen 501 and the secondary lumen 502 serving as the first pressure-filled lumen 503 and the second pressure-filled lumen 504.

As shown in FIG. 6, the main sheath tube 510 includes a first tip portion 506 adjacent to a distal end thereof, wherein a distal perforation of the main tubular lumen 501 is exposed to an end face of the first tip portion 506, and a distal perforation of the secondary tubular lumen 502 is exposed to a side face of the first tip portion 506. That is, the diagnostic instrument can be threaded through the end face of the first tip portion 506 of the main sheath 510 and the balloon catheter 700 can be threaded through the side of the first tip portion 506 of the main sheath 510. The design of the first tip portion 506 of the main sheath 510 reduces resistance to its traveling through the blood vessel, allowing for smoother access.

The material of the main sheath 510 and the secondary sheath 513 may include at least one of polyamide, polyether block polyamide, acrylonitrile-butadiene-styrene terpolymer, and polyethylene glycol p-toluene dicarboxylate.

The main sheath 510 is provided with at least one through-hole connecting the first pressure-filled lumen 503 and the second pressure-filled lumen 504 to the first balloon 511 (not shown in the drawings, e.g., the at least one through-hole may be formed by directly making an opening in the outer wall of the main sheath 510 opposite the first balloon 511). The first balloon 511 is a compliant wall-like balloon that fits the outer wall of the main sheath 510 in a contracted state. The compliant walled balloon is walled in the contracted state, and its proximal inner wall and distal inner wall can be hermetically bonded to the outer wall of the main sheath 510, thereby realizing a balloon seal.

The first balloon 511 is made of a compliant material, which may include, for example, at least one of silicone, latex, and polyurethane, so that a large expansion diameter can be obtained at a lower pressure, and the expansion diameter grows significantly as the pressure is elevated, so that it can be efficiently anchored after expansion to provide a reliable support in the blood vessel. In addition, the first balloon 511 adopts a tubular wall-like design, which fits with the outer wall of the main sheath 510 in the contracted state, so that a smaller outer diameter can be obtained, in order to minimize the resistance to travel in the blood vessel and reduce the harm to the human body.

In embodiments of the present disclosure, the material of the main connection seat 512 and the secondary connection seat 514 may include at least one of an acrylonitrile-butadiene-styrene terpolymer, a polyamide, a polycarbonate, and a polyformaldehyde. The primary attachment seat 512, and the secondary attachment seat 514 may provide access to other instruments or treatment media and may be made of a material that is stiff relative to the primary sheath 510 and the secondary sheath 513, so as to facilitate assembly and instrument manipulation. In addition, a hemostatic valve may be installed therein.

As shown in FIG. 6, the balloon sheath 600 further includes a primary hemostatic valve 591 disposed within the primary attachment seat 512, and a secondary hemostatic valve 592 disposed within the secondary attachment seat 514. The main hemostatic valve 591 and the vice hemostatic valve 592 utilize their own elasticity to squeeze and seal, preventing blood from flowing out and realizing a hemostatic effect. The materials of the main hemostatic valve 591 and the secondary hemostatic valve 592 may, for example, include at least one of silicone, latex, and polyurethane.

The first side branch tube 515, the second side branch tube 516, and the third side branch tube 517 are single lumen tubes that are bendable and capable of withstanding a certain amount of pressure, and can provide a pathway for various therapeutic mediums or flushing mediums, as well as collecting body fluids during treatment, and the materials thereof can, for example, include at least one of polyethylene, polyvinyl chloride, and polyether block polyamide.

The primary dilator 793 and the secondary dilator 794 serve as intermediate auxiliary instruments when placing the balloon sheath 600 and the balloon catheter 700. As shown in FIG. 6, the main structure of the main dilator 793, for example, includes a single lumen catheter 7931 and a dilator seat 7932, which serves to provide access for the guidewire to enter the main sheath 510. The single lumen catheter 7931 is provided with a tapering segment at the head end, so as to facilitate the assembly of the main dilator 793 with the main sheath 510 through the hemostatic valve 591. The material of the single lumen catheter 7931 may include at least one of polyethylene, polyvinyl chloride, and polyether block polyamide, and may also incorporate a metal oxide such as barium sulfate so as to be developable. The expander seat 7932 may comprise a luer fitting (not shown), which may be of a material consistent with the material of the single lumen catheter 7931.

Referring to FIG. 5, embodiments of the present disclosure also provide a heart failure treatment device 500 comprising the balloon sheath tube 600 of any of the preceding embodiments.

The heart failure treatment device 500 may include an associated diagnostic device, a balloon catheter 700, and an external device (e.g., a pump), among others. The heart failure treatment device 500 can be used not only for treatment protocols of venous occlusion, but also for treatment protocols of intra-aortic counter pulsation, which can lead to good prognostic results in the treatment of acute congestive heart failure.

The heart failure therapeutic device 500 further includes a balloon catheter 700 that is nested and assembled with the secondary sheath 513 and the primary sheath 510. The specific structural form of the balloon catheter 700 is not limited and can be specifically designed according to the actual needs.

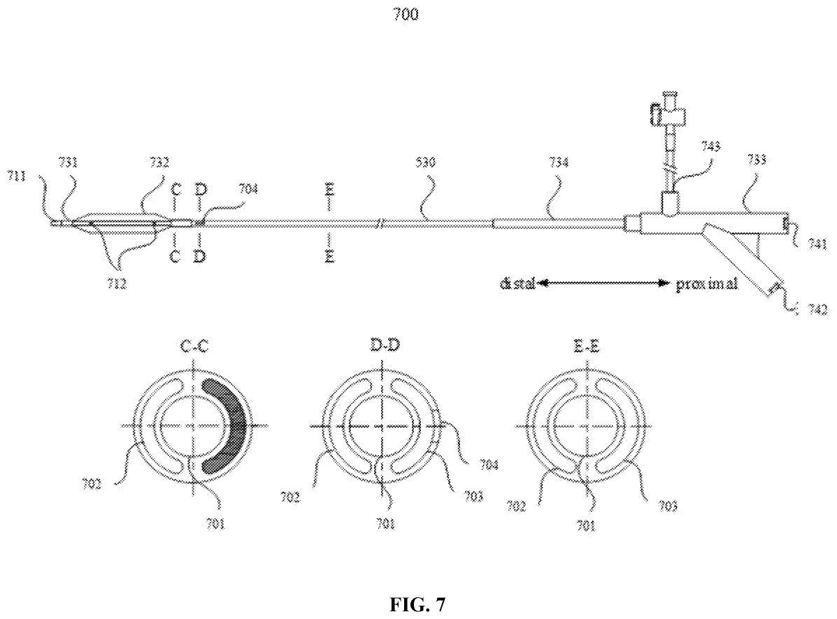

As shown in FIG. 7, the main structure of the balloon catheter 700 includes a multi-lumen tube 530, a single-lumen tube 531, a second balloon 532, and a connecting seat 533. the multi-lumen tube 530 has a guidewire feedthrough lumen 701, a pressure-filled media delivery lumen 702, and a contrast agent delivery lumen 703 that are not in communication with each other, as well as a contrast agent delivery lumen 703 that is exposed on the outer wall of the multi-lumen tube 530 and is distally connected to the contrast agent delivery lumen 703. The single lumen tube 731 is connected to the distal end of the guidewire penetration lumen 701. The second balloon 732 is sealingly connected to the outer wall of the multi-lumen tube 530 and the outer wall of the single lumen tube 731 and is connected to the distal end of the pressurized media delivery lumen 702. The coupling seat 733 is connected to the proximal end of the multi-lumen tube 530 and includes a first connector 741 communicating with the guidewire threading lumen 701, a second connector 742 communicating with the pressure-filled medium delivery lumen 702, and a third connector 743 communicating with the contrast agent delivery lumen 703, wherein the first connector 741 is used for threading of the guidewire, the second connector 742 is used for inputting the pressure-filled medium, and the third connector 743 is used for inputting the contrast agent. is used for inputting the contrast agent.

After the balloon catheter 700 is placed and positioned in the human body using the balloon sheath 600 provided in the preceding embodiment of the present disclosure, the first balloon 511 and the second balloon 732 can be alternately and intermittently filled using an external pressure-filling medium input device, thereby realizing alternately and intermittently blocking the inferior vena cava and the subclavian vein as a means of reducing the venous blood flow back to the heart, or, alternatively, the first balloon 511 and the second balloon 732 are synchronously inflated and deflated in accordance with cardiac rhythms as a means of elevating diastolic pressure and coronary perfusion, and reducing cardiac afterload to improve left ventricular ejection. Additionally, an external contrast fluid input device may be utilized to allow the contrast fluid to be injected into the blood vessel through the contrast delivery lumen 703. The contrast delivery lumen 704 may be designed on a side of the second balloon 732 away from the single lumen tube 731.

The specific form of construction within the multi-lumen tube 530 is not limited. As shown in FIG. 7, the guidewire entry lumen 701 is circularly perforated and its center axis coincides with the center axis of the multi-lumen tube 530, and the pressure-filled medium delivery lumen 702 and the contrast agent delivery lumen 703 are each curved perforations surrounding the guidewire entry lumen 701. The material of the multi-lumen tube 530 may include at least one of a polyamide or a polyether-blocked polyamide.

As shown in FIG. 7, the single lumen tube 731 includes a second tip portion 711 adjacent to its distal end, which facilitates reducing the resistance of the balloon catheter 700 as it travels through the blood vessel, allowing for smoother entry. The single lumen tube 731 may be made of a softer material, and may for example comprise at least one of polyurethane, polyimide, polyether block polyamide, and silicone.

The outer wall of the single lumen tube 731 may be provided with a developing element 712, and the developing element 712 may be designed within the second balloon 732. During surgery, the developing element 712 can be detected using an external image detection device (e.g., an X-ray detection device) so that its position can be accurately known and precise positioning can be realized. The material of the developing element 712 may be selected from one or more of gold, platinum, iridium, tantalum, tungsten, and other materials.

The second balloon 732 is a non-compliant balloon, the material of which may include at least one of polyamide, polyether block polyamide, and polyethylene. The diameter of the non-compliant balloon, after expanding to a certain value, will remain at that value regardless of changes in external pressure, which allows for precise sealing of the blood vessel at the specified location without damaging the blood vessel due to over-expansion.

As shown in FIG. 7, the balloon catheter 700 further comprises: a reinforcing sleeve 734 that is provided at the connection between the multi-lumen tube 530 and the connection seat 733 and seals the multi-lumen tube 530 to the connection seat 733. The reinforcing sleeve 734 is used to seal the connection between the multi-lumen tube 530 and the connection seat 733 firmly, and also prevents excessive bending at the connection. The material of the reinforcing sleeve 734 may include at least one of a polyamide, an acrylonitrile-butadiene-styrene terpolymer, a polyolefin, a polyether block polyamide, and the like, and may also include a metal oxide such as barium sulfate.

The heart failure treatment device 500 may be used for treatment implementation according to some embodiments of the present disclosure as follows.

First, a channel is established in the femoral vein by means of a puncture kit and a guidewire is placed. Then, a balloon sheath tube 600 is introduced into the body from the femoral vein along a guidewire (the guidewire is threaded into the main lumen 501 of the balloon sheath tube 600), and the first balloon 511 is positioned under contrast to the inferior vena cava, which is located underneath the renal vein, to secure the balloon sheath tube 600. Then, a guidewire is placed in the secondary lumen 502 of the balloon sheath tube 600, so that the guidewire passes upwardly along the inferior vena cava through the heart, the superior vena cava, and the cephalic arm vein to the subclavian vein. Then, the balloon catheter 700 is placed along the guidewire through the secondary lumen 502 to reach the designated position, and in the process, the contrast agent is delivered into the blood vessel through the contrast agent delivery holes 704 which are provided with the balloon catheter 700, so as to monitor that the second balloon 732 accurately arrives at the blocking position.

Thereafter, the balloon sheath 600 and the balloon catheter 700 are connected to pressure pumps, respectively, to begin a cyclic treatment process comprising the following four steps:
Step I. The first balloon 511 completes pressure filling in 4-5 s, and the pressure is maintained for 15 s. At this time, the second balloon 732 remains unpressurized;
Step II, the first balloon 511 completes pressure relief in 4-5 s to allow blood flow for 1 minute;
Step III, the second balloon 732 completes filling the pressure in 4-5 s, and the pressure is maintained for 15 s, at which time the first balloon 511 is maintained in a non-pressurized state;
Step IV, the second balloon 732 completes the pressure relief in 4-5 s to allow the blood to flow for 1 minute, at which time the first balloon 511 remains unpressurized.

The cycle repeats the above treatment process several times until the end of treatment. Through the above treatment process, the return of venous blood to the heart can be slowed down and the preload of the heart can be reduced.

During the treatment process or before and after the treatment, the main lumen 501 can be passed into the diagnostic device for relevant testing and treatment, and the diagnostic device can be, for example, a floating catheter, an ablation catheter, and so on.

As can be seen, for the treatment option of venous occlusion, the veins can be occluded by two balloons with intermittent pressure filling and pressure relief, thereby reducing venous return to the heart and reducing the preload of the heart, and the main lumen 501 can provide access for other diagnostic and therapeutic devices.

The heart failure treatment device 500 may be used for a second therapeutic treatment as follows:

First, a channel is established in the femoral artery by means of a puncture kit and a guidewire is placed. Then, the balloon sheath tube 600 is introduced into the body from the femoral artery along the guidewire (which is threaded into the main lumen 501 of the balloon sheath tube 600), and the first balloon 511 is positioned under the aortic arch under the angiography, which is located in the thoracic aorta, to secure the balloon sheath tube 600. Then, the collateral lumen 502 of the balloon sheath tube 600 is placed with the guidewire, so that the guidewire enters one of the left subclavian artery, the common carotid artery, and the trunk artery of the head and arm along the femoral artery (depending on the clinical assessment). Then, the balloon catheter 700 is passed along the guidewire through the secondary lumen 502 to reach the designated position, and in the process, the contrast agent is delivered to the vessel through the contrast agent delivery holes 704 that are provided with the balloon catheter 700, so as to monitor that the second balloon 732 accurately arrives at the blockage position.

After that, the balloon sheath tube 600 and the balloon catheter 700 are connected to pressure pumps, respectively, to start the following treatment process.

When the heart is in diastole, the first balloon 511 and the second balloon 732 are filled and blocked, and when the heart is in systole, the first balloon 511 and the second balloon 732 are deflated, resulting in a dual hemodynamic effect: filling the two balloons with pressure in diastole to bring the blood flow forward, which improves the diastolic blood pressure and coronary perfusion; and deflating the two balloons in systole to reduce the systolic blood pressure (cardiac afterload), which improves left ventricular Ejection. The first balloon 511 and the second balloon 732 can be inflated and deflated by the pressure pump once every cardiac cycle (according to the 1:1 mode), once every two cardiac cycles (according to the 1:2 mode), and once every three cardiac cycles (according to the 1:3 mode).

During or before or after the treatment, the main lumen 501 can be passed into a diagnostic device for relevant testing and treatment, which can be, for example, a coronary balloon, a coronary stent, etc.

As can be seen, for the treatment of intra-aortic counter pulsation, the two balloons can be used to simultaneously inflate and deflate in accordance with the cardiac rhythm, thereby generating a dual hemodynamic effect, increasing diastolic blood pressure and coronary perfusion, decreasing the cardiac afterload, and improving the left ventricular ejection; in addition, the main lumen 501 can provide access to other diagnostic and therapeutic devices.

### Example 4: Exemplary Nested Multi-Balloon Catheter Devices

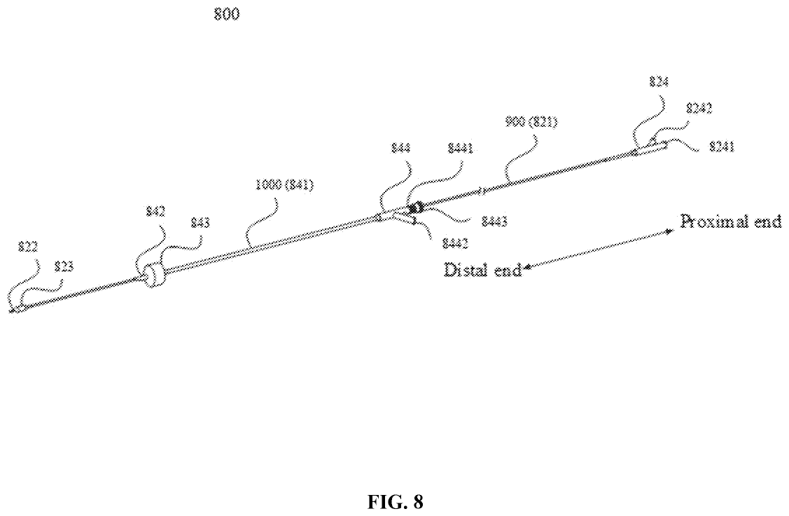

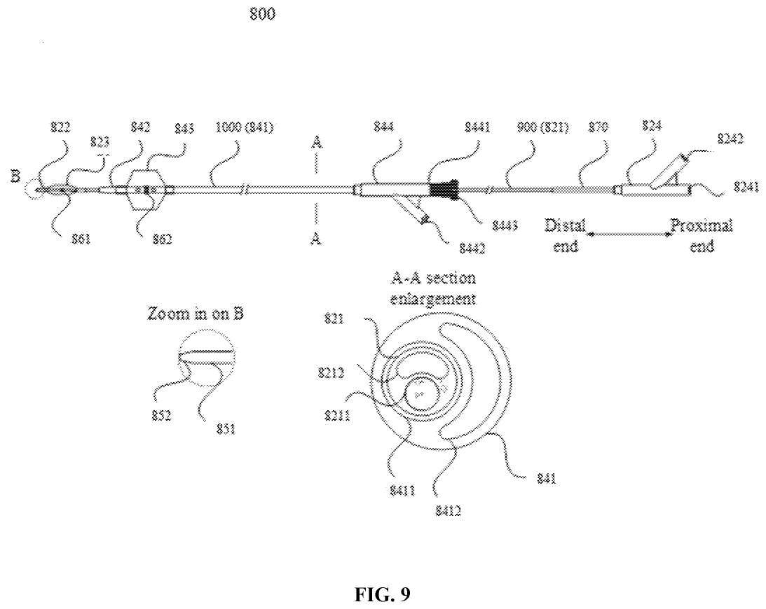

In FIG. 8 and FIG. 9, the catheter device 800 provided in some embodiments of the present disclosure includes an inner balloon catheter 900 and an outer balloon catheter 1000 that are nested and assembled.

The inner balloon catheter 900 includes a first multi-lumen tube 821, a first single-lumen tube 822, a first balloon 823 and a first hub 824. The first multi-lumen tube 821 has a first lumen 8211 and a second lumen 8212 that are not connected to each other. The first single-lumen tube 822 is connected to the distal end of the first lumen 8211. The first balloon 823 is sealedly connected to the outer wall of the first multi-lumen tube 821 and the outer wall of the first single-lumen tube 822 and is connected to the distal end of the second lumen 8212. The first hub 824 is connected to the proximal end of the first multi-lumen tube 821 and includes a first connector 8241 connected to the first lumen 8211 and a second connector 8242 connected to the second lumen 8212, wherein the first connector 8241, the first lumen 8211 and the first single-lumen tube 822 are used for the guide wire to penetrate, and the second connector 8242 and the second lumen 8212 are used for conveying a pressurized medium.

The outer balloon catheter 1000 includes a second multi-lumen tube 841, a second single-lumen tube 842, a second balloon 843, and a second hub 844. The second multi-lumen tube 841 has a third lumen 8411 and a fourth lumen 8412 that are not connected to each other. The second single-lumen tube 842 is connected to the distal end of the third lumen 8411. The second balloon 843 is sealed and connected to the outer wall of the second multi-lumen tube 841 and the outer wall of the second single-lumen tube 842 and is connected to the distal end of the fourth lumen 8412. The second hub 844 is connected to the proximal end of the second multi-lumen tube 841 and includes a third connector 8441 connected to the third lumen 8411, a fourth connector 8442 connected to the fourth lumen 8412, and a locking structure 8443 arranged at the proximal end of the third connector 8441, wherein the third connector 8441, the third lumen 8411, and the second single-lumen tube 842 are used for the inner balloon catheter 900 to penetrate, the fourth connector 8442 and the fourth lumen 8412 are used to transport pressurized medium, and the locking structure 8443 is used to lock the relative sliding position of the inner balloon catheter 900 in the outer balloon catheter 1000.

In the embodiment of the present disclosure, the end of the catheter device 800 or its components that is closer to the operator in the extension direction is defined as the “proximal end”, and the “proximal end” should be understood in a broad sense, including the “proximal end face” and a portion of the structure adjacent to the “proximal end face”. Similarly, the end of the catheter device 800 or its components that is farther away from the operator in the extension direction is defined as the “distal end”, and the “distal end” should be understood in a broad sense, including the “distal end face” and a portion of the structure adjacent to the “distal end face”.

The catheter device 800 can be placed into the human body with the aid of a guide kit (including but not limited to a puncture needle, a sheath, a guide wire, etc.). The outer balloon catheter 1000 is used to enter the inferior vena cava, and the inner balloon catheter 900 is used to pass through the outer balloon catheter 1000 and go up along the vein to enter the subclavian vein. During the operation, first, under the guidance of the guide kit, the outer balloon catheter 1000 is introduced into the human body from the femoral vein, and the second balloon 843 is positioned in the inferior vena cava under angiography, and its position is located below the renal vein. Then, under the guidance of the guide kit, the inner balloon catheter 900 is passed through the third connector 8441, the third lumen 8411 and the second single lumen 842 to pass through the outer balloon catheter 1000, and then enter the subclavian vein upward along the venous blood vessels, so that the first balloon 823 is positioned in the subclavian vein under angiography. Then, the relative positions of the inner balloon catheter 900 and the outer balloon catheter 1000 are locked by the locking structure 8443. Thereafter, the inner balloon catheter 900 and the outer balloon catheter 1000 are respectively connected to the pressure pumps, and the treatment process of intermittent filling and blocking of the superior and inferior vena cava blood is started.

According to the design of the embodiment of the present disclosure, the double balloons of the catheter device 800 can be used to intermittently block the veins, thereby reducing the venous blood backflow to the heart, reducing the precardiac load, and reducing the stress on the ventricular wall. It can be used to treat acute congestive heart failure and can achieve a good prognosis.

In FIG. 9, the inner balloon catheter 900 further includes a first developing element 861 disposed on the outer wall of the first single-lumen tube 822 and located in the first balloon 823, and the outer balloon catheter 1000 further includes a second developing element 862 disposed on the outer wall of the second single-lumen tube 842 and located in the second balloon 843. During surgery, the developing element can be detected by an external image detection device (such as an X-ray detection device) to accurately know its position and achieve accurate positioning of instrument intervention. The material of the above-mentioned developing element can be selected from one or more materials such as gold, platinum, iridium, tantalum, and tungsten.

The material of the first hub 824 and the second hub 844 includes at least one of polyamide, polycarbonate, and polyoxymethylene. The connection seat can provide an inlet and outlet channel for a guide wire, a catheter, and treatment-related media, and its material is relatively hard, which is convenient for assembly and instrument operation. In addition, a hemostatic valve can be installed therein.

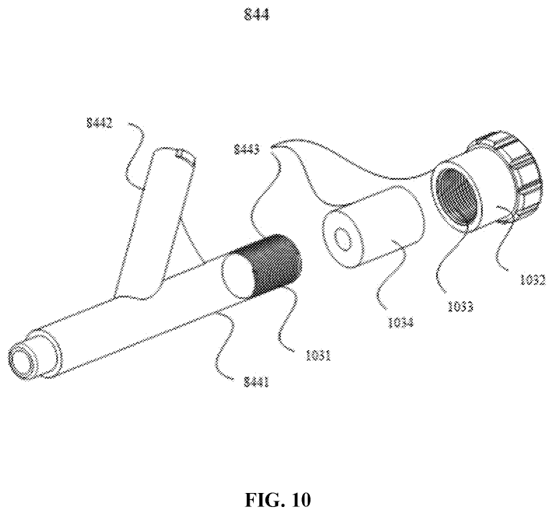

The locking structure 8443 is used to reliably lock the relative sliding position of the inner balloon catheter 900 in the outer balloon catheter 1000, and its specific structural form is not limited. As shown in FIG. 10, the locking structure 8443 includes: an external thread segment 1031 provided at the proximal end of the third connector 8441, a gland 1032 having an internal thread segment 1033 connected to the external thread segment 1031, and a hemostatic valve 1034 provided on the inner side of the external thread segment 1031 and having a through hole for the inner balloon catheter 900 to pass through.

The hemostatic valve uses its own elastic force to closely contact with other components, thereby squeezing and sealing to prevent blood from flowing out and achieve a hemostatic effect. In the disclosed embodiment, the material of the hemostatic valve 1034 may include, for example, at least one of silicone, latex, and polyurethane. When the gland 1032 is tightened, the hemostatic valve 1034 presses the inner balloon catheter 900 to lock its relative sliding position in the outer balloon catheter 1000. The locking structure 8443 is simple in design, easy to operate, and can achieve reliable locking.

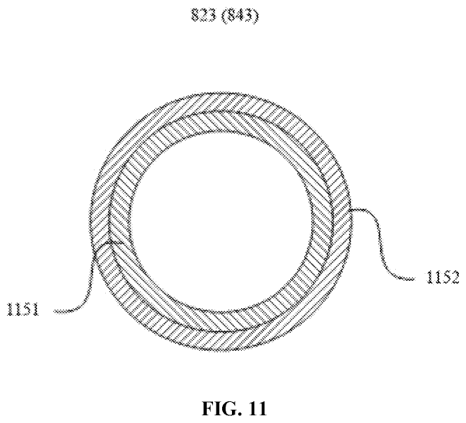

In FIG. 11, the first balloon 823 and the second balloon 843 are double-layer balloons, respectively, and the double-layer balloons include an outer layer material 1152 and an inner layer material 1151 that are attached to each other, wherein the hardness of the outer layer material 1152 is greater than the hardness of the inner layer material 1151. The outer layer material 1152 may include, for example, at least one of polyamide, polyether block polyamide, and polyethylene terephthalate, and the inner layer material 1151 may include, for example, at least one of polyether block polyamide, polyurethane, thermoplastic elastomer, silicone, and latex.

The double-layer balloon can maintain a relatively small, contracted volume when not under pressure, and can maintain a stable expanded volume when under pressure within a certain range. The double-layer balloon can accurately block the blood vessels at the designated location and will not damage the blood vessels due to excessive expansion. It also has better puncture resistance, thus greatly reducing the risk of rupture.

In FIG. 9, the first lumen 8211 of the inner balloon catheter 900 is in the shape of a circular hole, and the second lumen 8212 is in the shape of a crescent hole, wherein the central axis of the first lumen 8211 (as shown by point P in the figure) is eccentrically arranged relative to the central axis of the first multi-lumen tube 821 (as shown by point O in the figure). The third lumen 8411 of the outer balloon catheter 1000 is in the shape of a circular hole, and the fourth lumen 8412 is in the shape of a crescent hole, wherein the central axis of the third lumen 8411 (as shown by point O in the figure) is eccentrically arranged relative to the central axis of the second multi-lumen tube 841 (as shown by point Q in the figure). In this way, the first lumen 8211 facilitates the passage of a guide wire with a circular cross section, and the third lumen 8411 facilitates the passage of the inner balloon catheter 900 with a circular cross section outer contour, so that the entry operation of the catheter device 800 in the human body is smoother.

The material of the first multi-lumen tube 821 and the second multi-lumen tube 841 may include at least one of polyamide, polyether block polyamide, polyurethane, polyethylene, and polypropylene.

In FIG. 9, the first single-lumen tube 822 and the second single-lumen tube 842 both include a body portion 851 and a tip portion 852 connected to the distal end of the body portion 851 (FIG. 9 only illustrates a partial enlarged structure of the first single-lumen tube 822).

The material of the body portion 851 may include at least one of polyamide and polyether block polyamide. The tip portion 852 may be made of a softer material than the body portion 851, for example, it may include at least one of polyurethane, polyether block polyamide, and silicone. The tip portion 852 may be welded to the body portion 851. The material selection and shape design of the tip portion 852 may reduce the resistance of the catheter device 800 in the blood vessel, making the entry smoother.

As in FIG. 9, the catheter device 800 further includes a reinforcing sleeve 870, which is sleeved at the connection between the first multi-lumen tube 821 and the first hub 824 and seals and connects the first multi-lumen tube 821 to the first hub 824. The reinforcing sleeve 870 is used to make the connection between the first multi-lumen tube 821 and the first hub 824 seal firmly, and also to prevent excessive bending at the connection. The material of the reinforcing sleeve 870 may include at least one of polyamide, acrylonitrile-butadiene-styrene terpolymer, polyolefin, polyether block polyamide, etc., and may also include metal oxides such as barium sulfate.

The disclosed embodiment also provides a heart failure treatment device, comprising the catheter device 800 of any of the aforementioned embodiments.

The heart failure treatment device may also include a guide kit, a pressure pump, a pressure detection device, etc., which are not specifically limited in the present disclosure. The heart failure treatment device is used in a venous occlusion treatment scheme, so that a good prognosis can be obtained in the treatment of acute congestive heart failure.

The heart failure treatment device may be used for therapeutic implementation as follows:

First, under the guidance of the guide kit, the outer balloon catheter 1000 is introduced into the human body from the femoral vein, and the second balloon 843 is positioned in the inferior vena cava under angiography, and its position is below the renal vein. Then, under the guidance of the guide kit, the inner balloon catheter 900 is passed through the third connector 8441, the third lumen 8411 and the second single-lumen tube 842 to pass through the outer balloon catheter 1000, so that the first balloon 823 is positioned in the subclavian vein under angiography, and the relative sliding position of the inner balloon catheter 900 in the outer balloon catheter 1000 is locked by the locking structure 8443.

Afterwards, the outer balloon catheter 1000 and the inner balloon catheter 900 are connected to the pressure pumps respectively, and the cyclic treatment process including the following four steps is started:
Step 1: The second balloon 843 is pressurized in 4-5 seconds, and the pressure is maintained for 15 seconds, while the first balloon 823 remains unpressurized;
Step 2: The second balloon 843 is depressurized within 4-5 seconds, allowing blood to circulate for 1 minute;
Step 3: The first balloon 823 is pressurized in 4-5 seconds, and the pressure is maintained for 15 seconds, while the second balloon 843 remains unpressurized;
Step 4: The first balloon 823 is depressurized in 4-5 seconds, allowing blood to circulate for 1 minute, while the second balloon 843 remains in a non-pressurized state.

Repeat the above treatment process several times until the treatment is completed. Through the above treatment process, the return of venous blood to the heart can be slowed down and the precardiac load can be reduced.

It can be seen that for the treatment of venous occlusion, the veins can be blocked by using two balloons in combination with intermittent inflation and decompression, thereby reducing the return of blood from the veins to the heart, alleviating the precardiac load, and achieving a good prognosis.

Since the catheter device 800 may be used in the human body for up to 12 hours, and the use environment may not be a sterile environment, considering this scenario, the product can be equipped with a vascular sheath and a sterile sheath (not shown in the figure) when designing. The vascular sheath is coaxially sleeved on the outside of the second multi-lumen tube of the outer balloon catheter and is composed of a sheath tube and a seat. One end of the sterile sheath is connected to the seat of the vascular sheath, and the other end is connected to the second hub of the outer balloon catheter. It can be extended and retracted as the length of the device entering the human body, so as to keep this section of the catheter in a sterile state for a long time and avoid infection as much as possible.

A sterile sheath (not shown in the figure) can also be installed between the locking mechanism of the outer balloon catheter and the first hub of the inner balloon catheter. The length of the sterile sheath can be expanded or contracted as the relative positions of the inner balloon catheter and the outer balloon catheter change, thereby keeping this section of the catheter in a sterile state for a longer period of time and avoiding infection as much as possible.

### Example 5: Animal Study of Balloon Catheter System

An animal study was designed to comprehensively evaluate the safety and efficacy of the catheter system and determine optical clinical settings. The investigational device comprises a support device, multi-lumen catheter, and single lumen catheter. The multi-lumen balloon catheter is designed for inferior vena cava balloon occlusion, inferior vena cava pressure monitoring, and as a sheath for the subclavian balloon catheter. The single-lumen catheter is designed for subclavian vein balloon occlusion and superior vena cava pressure monitoring. The catheters were placed in the infra-renal inferior vena cava and left subclavian vein, and occlusion parameters were controlled via the support system.

Nine adult pigs were used in this study. The test animals weighed from 40 to 72 kg and the animals were housed in appropriately sized cages, fed twice daily, and had free access to water. Standard drugs were used for animal anesthesia, intraoperative hemodynamic maintenance, and euthanasia procedures. Routine reagents and consumables were used for necropsy and histopathological examination.

A heart failure model was established using a combination of ischemic cardiomyopathy and valvular regurgitation. Acute mitral regurgitation and coronary artery balloon embolization were performed sequentially. The heart failure model was characterized by a left ventricular ejection fraction and heart enlargement. Heart failure model can be further characterized by myocardial infarction and mitral valve damage. Four subjects were healthy and five were heart failure models.

Hemodynamic data was collected of the subject during testing. Heart rate (HR) and arterial blood pressure (ABP) were measured via arterial catheter connected to invasive pressure sensors; central venous pressure (CVP), pulmonary artery pressure (PAP), pulmonary capillary wedge pressure (PCWP), cardiac output (CO), subclavian vein pressure (SCVP), and inferior vena cava pressure (IVCP) were measured using a Swan-Ganz floating catheter; left ventricular ejection fraction (LVEF) and left atrial end-diastolic volume (LAEDV) were assessed by transthoracic echocardiography. Hemodynamic data was measured every 1 minute after treatment initiation, with optional longer intervals (5 minutes, 10 minutes, 15 minutes, etc.).

Necropsy and histopathological examinations were performed to assess injury and hemorrhage at the balloon dilation sites, presence of thrombi within the balloon dilation sites and upstream and downstream vessels, distal tissue damage, and pathological conditions of the vascular layers at the balloon dilation sites.

The occlusion protocol was as followed: 1) Establish vascular access via the femoral vein of the subject and insert a catheter guidewire through this access; 2) Perform venography of the superior vena cava (SVC) and inferior vena cava (IVC) to determine their anatomical positions and courses. The balloon placement site in the infrarenal IVC is approximately at the L1-L2 vertebral level, about 10 mm to 15 mm from the right renal vein. The balloon site in the left subclavian vein is approximately at the C7-T1 vertebral level, near the confluence of the left internal jugular vein and the left subclavian vein; 3) Measure the diameter of the vessels at the intended balloon placement sites; 4) Insert the balloon catheters into the corresponding positions. After balloon inflation, inject contrast medium through the vascular sheath and observe under DSA to confirm balloon position and the degree of vascular occlusion, and determine balloon inflation volumes corresponding to 100%, 85%, and 70% occlusion; 5) Insert a Swan-Ganz catheter via the jugular vein to measure baseline and intra-treatment hemodynamic parameters; 6) Connect the balloon catheters to the Circulatory Assist and Regional Perfusion System. After setting the parameters for balloon occlusion and release, the system automatically performs inflation and deflation; and 7) Remove all catheters after the trial conclusion, where the trial lasts up to 8 hours or ends upon reaching termination criteria.

Various modes were tested, where each mode was intended to vary based on occlusion rate and cycle. The occlusion rate was the percentage of vein occlusion taking place by the balloon, as a percentage. The cycle was the amount of time when vein occlusion took place vs. time when veins were not occluded. During the testing, multiple different occlusion parameters were tested, including occlusion times for each balloon, occlusion percent of the vein, synchronous occlusion of the first and second balloon, and the overall pre-determined test time. The occlusion time for each balloon ranges from 0 minutes to 20 minutes. The occlusion percent of the vein, when occlusion was present, ranged from 70% to 100%. The synchronous occlusion time 0% to 100% of the time. The overall pre-determined time ranged from 1 minute to 21 minutes.

During surgery on the test animals, the circulatory shunting support system operated stably. The tests could automatically control balloon inflation and release based on preset programming. Further, after incomplete balloon occlusion, there was no indication of thrombosis formation, vascular perforation, vascular stripping, hematoma, or other abnormalities.

Isolated occlusion of the intra-renal IVC significantly increased distal IVC pressure. The IVC balloon was inflated at 6 mL/sec with an inflation volume of 5 mL for a 21 mm diameter balloon.

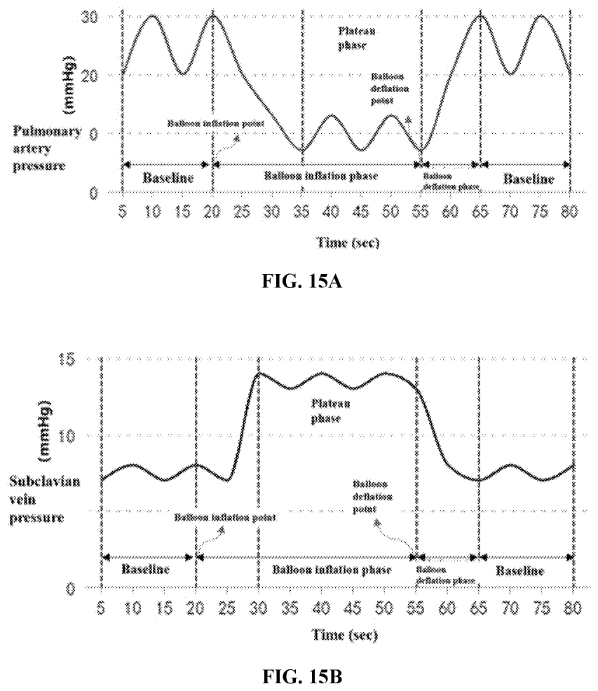

Simultaneous occlusion of the infra-renal IVC and subclavian vein significantly reduced pulmonary artery pressure. When simultaneously occluding the infra-renal IVC (Balloon: 6 mL volume, 22 mm diameter) and the subclavian vein (Balloon: 0.6 mL, 10 mm diameter), pulmonary artery pressure decreases from the baseline as the balloons inflate, reaching a plateau after 15 seconds. Upon balloon release, the pressure rapidly recovers and returns to baseline within 10 seconds (FIG. 15A). Here, the baseline pressure was around 15-35 mmHg and reached a plateau around 5-20 mmHg. Conversely, the subclavian vein pressure shows the opposite trend, increasing from a baseline with balloon inflation, reaching a plateau after 15 seconds. Upon balloon release, it rapidly decreases and returns to baseline within 10 seconds (FIG. 15B). Here, the baseline pressure was around 5-15 mmHg and reached a plateau around 10-25 mmHg.

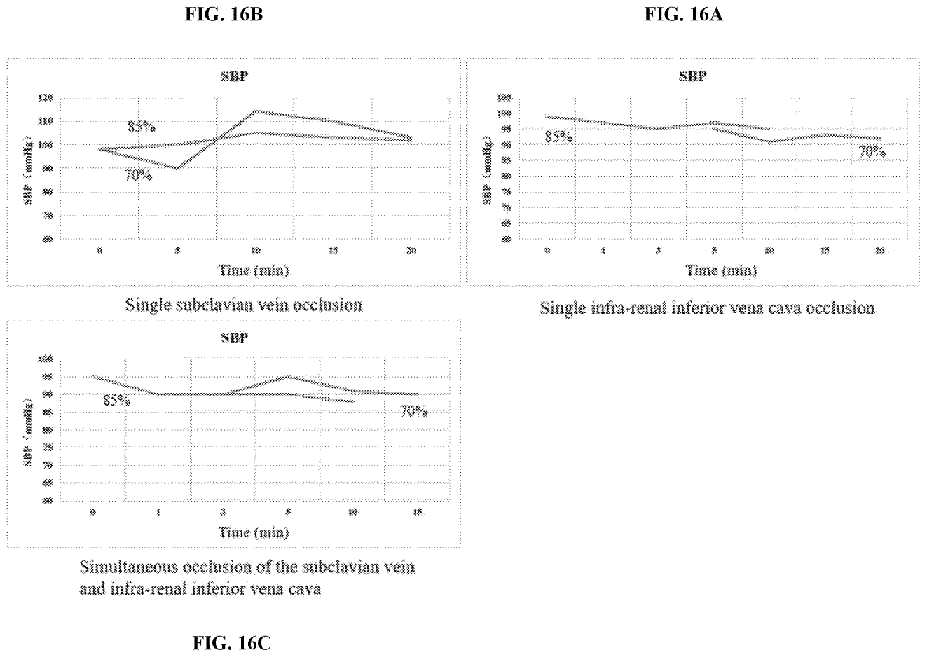

Intermittent partial occlusion of the subclavian vein and infrarenal IVC provides hemodynamic stability. By occluding the subclavian vein and the infrarenal inferior vena cava at various levels below 100%, respectively or simultaneously, the hemodynamic changes in the test animals and the alleviation of heart failure symptoms were observed, showing partial occlusion had little effect on hemodynamics. Specifically, single subclavian vein occlusion at 70% and 85% for 20 minutes caused arterial systolic pressure fluctuations between 90 mmHg (baseline) and 120 mmHg, with an overall upward trend in blood pressure during treatment. Single inferior vena cava occlusion at 75% for 20 minutes and 85% for 10 minutes caused arterial systolic pressure fluctuations between 110 mmHg (baseline) and 85 mmHg. These results are shown in FIGS. 16A-16C for systolic blood pressure over 70% and 85% occlusion. Therefore, partial vessel occlusion has minimal impact on hemodynamics.

Hemodynamic fluctuations were found to be related to the frequency of intermittent balloon occlusion and intermittent partial occlusion of the subclavian vein and infra-renal IVC provides hemodynamic stability. The HR and PCWP of the heart failure animals tended to decrease when the subclavian vein was partially occluded, while LVEF and LAD tended to increase with improved cardiac function at higher occlusion. For partial occlusion of the infrarenal inferior vena cava, similar occlusion levels as utilized in the subclavian vein were shown to improve cardiac function to a similar degree with no significant difference between the different levels, wherein the levels are from about 50% to about 95%.

After determining the appropriate occlusion size, experimentation was completed to determine optimal occlusion cycle for intermittent partial occlusion. The test animals repeatedly experienced a decrease in arterial systolic pressure exceeding 10% when balloons were simultaneously occluded, where one was fully occluded. Accordingly, only simultaneous occlusion with two balloons caused hemodynamic instability, wherein asynchronous partial occlusion of the subclavian vein and infrarenal inferior vena cava was determined to be necessary. Applying asynchronous partial occlusion allowed for stable hemodynamics with conventional fluid administration and intravenous vasoactive drugs and increased cardiac output and left ventricular ejection fraction, along with decreased pulmonary capillary wedge pressure and left atrial volume suggesting that this balloon inflation and deflation mode is both safe and effective.

After testing, the catheter device showed enhanced performance in terms of insertion, withdrawal, balloon inflation, and deflation. Additionally, the graphical interface of the circulatory flow diversion support device was optimized, improving data refresh rate and display completeness. Further, repeated balloon inflation, even without direct vessel wall contact, can cause local pressure-related injury.

Overall, vascular damage and thrombosis remained controllable, and no embolic events were observed in the treated vessels or downstream organs. Histopathology revealed no abnormalities in the subclavian vein at the site of balloon expansion and localized effect in the IVC.

Ultimately, the study provided a number of results. First, the circulatory flow diversion support device and balloon catheter exhibited favorable performance. Specifically, the improved balloon catheter showed enhanced performance in terms of insertion, withdrawal, balloon inflation, and deflation and provided stable operation.

Second, intermittent partial balloon occlusion demonstrated better safety. Through repeated testing of various occlusion modes (including complete occlusion of a single vein (subclavian or infra-renal IVC), simultaneous complete occlusion of both veins, intermittent complete occlusion of both veins, partial occlusion of a single vein, simultaneous partial occlusion of both veins, and intermittent partial occlusion of both veins), both continuous and intermittent complete occlusions resulted in hemodynamic instability. In contrast, partial occlusion resulted in relatively stable hemodynamics. Minor drops in blood pressure could be corrected with small doses of vasopressors and fluids, aligning with prior trials, suggesting that intermittent partial occlusion holds potential for clinical application.

Additionally, study on intermittent partial occlusion showed that intermittent occlusion of the subclavian vein combined with occlusion of the infra-renal inferior vena cava improved cardiac output and left ventricular ejection fraction, while reducing pulmonary capillary wedge pressure and left atrial volume. This configuration also maintained the stable hemodynamics over the entire 8-hour trial period, suggesting that it could effectively improve acute heart failure.

Third, long-term vascular injury after treatment was generally controllable. Under intermittent partial occlusion, no thrombus adhesion was observed on the balloon surface, and no significant thrombus formation was seen in distant organs such as the heart and kidneys. This indicates that the anticoagulation strategy was effective in preventing catheter-related thrombosis without inducing vascular bleeding at the catheter site. Histological analysis 30 days post-treatment revealed localized vascular injury, inflammatory cell infiltration, and mural thrombus formation in the infra-renal IVC and renal veins. These findings suggest that repeated balloon inflation, even without direct vessel wall contact, can cause local pressure-related injury. However, such vascular remodeling-including cell infiltration and thrombosis-are physiologically expected during healing. Overall, vascular damage and thrombosis remained controllable, and no embolic events were observed in the treated vessels or downstream organs.

Ultimately, the study successfully optimized a triple-balloon catheter system, enhancing both performance and safety. An intermittent occlusion strategy involving 85% subclavian vein occlusion combined with 70% infra-renal IVC occlusion produced minimal hemodynamic disturbance, achieved optimal therapeutic effects for acute heart failure, and demonstrated favorable acute and long-term safety.

### Example 6: Evaluation of Effects of the Circulation Decongestion Support System in a Porcine Model

An animal study was designed to evaluate a catheter system that can control the inflation and deflation of balloon catheters. ECG and blood pressure was also monitored.

Two swine were utilized for treatment, wherein Animal A survived for 27 days post procedure and Animal B was euthanized on the procedure date due to poor prognosis of respiratory distress.

Endpoint 1 for this study was regarding overall animal health (moribundity). Specifically, the endpoint assessed clinical observations, clinical pathology, and medical treatments while the animal was enrolled on study. No success criteria was defined for endpoint 1, results are summarized as follows. Animal B was euthanized during the recovery period on Day 0 due to poor prognosis, with evidence of respiratory distress observed during gross necropsy procedures.

Veterinary assessments of the animal surviving to the terminal time point (Animal A) inclusive of physical examinations, body condition scores, body weights, clinical monitoring and clinical pathology were performed from prior to Day 0 through termination with all assessments performed or reviewed by a Test Facility Veterinarian. All assessments suggested that animal remained in good general health throughout the duration of the study.

Endpoint 2 for this study was regarding local tissue response to the Test Article. Specifically, the study assessed the local vasculature to determine the occurrence of stenosis and vascular malfunction. The success criteria for this parameter of Endpoint 2 were no stenosis and malfunction of the local vasculature. The IVC and SCV were measured at baseline and termination for Animal A to determine stenosis of the vasculature. The measurements for baseline measurements of both vessels are comparable to the termination measurements, indicating no stenosis of the vessel. The vasculature was patent and normal for both. Success criteria were met for this portion of Endpoint 2.

Additionally, the Test Article-treated tissues and other potentially affected tissues were assessed grossly for abnormalities, and tissue responses to the Test Article were assessed by the Study Pathologist to determine the following: alterations of the venous treatment sites, upstream effects of the treatment and Test Article, downstream effects of the treatment and Test Article including the presence of thromboembolic effects and embolic particulates, and systemic effects of the treatment and Test Article. No success criteria were defined for these parameters of Endpoint 2. Results from necropsy and histopathology indicated no unexpected trauma or alteration of the venous treatment sites for the timepoint of necropsy, and there was no evidence of downstream thromboembolism or lesions associated with the test article.

All Endpoints were evaluated per protocol for this study. No success criteria was defined for endpoint 1 (overall animal health), however, in summary, one animal was terminated during the recovery period due to poor prognosis and respiratory distress; the other animal was terminated at 27 days per protocol and was in overall good health throughout the in-life period. Endpoint 2 (local tissue response to the test article) had success criteria for vascular stenosis and malfunction of the local vasculature, which was met. No success criteria was defined for the other aspect of endpoint 2, alterations of the venous treatment sites, upstream effects of the treatment and Test Article, downstream effects of the treatment and Test Article including the presence of thromboembolic effects and embolic particulates, and systemic effects of the treatment and Test Article. For this aspect of endpoint 2 there were no unexpected findings. Overall, based on the procedures performed and data collected on this study, the Test Articles: Circulation Decongestion Support Device, Multi-lumen Occlusion Balloon Catheter, and Single-lumen Occlusion Balloon Catheter performed as expected with regards to overall animal health and local tissue response in this chronic in vivo swine model.

### Example 7: Exemplary Occlusion Schematic for Treatment of Heart Failure Using Three Balloons

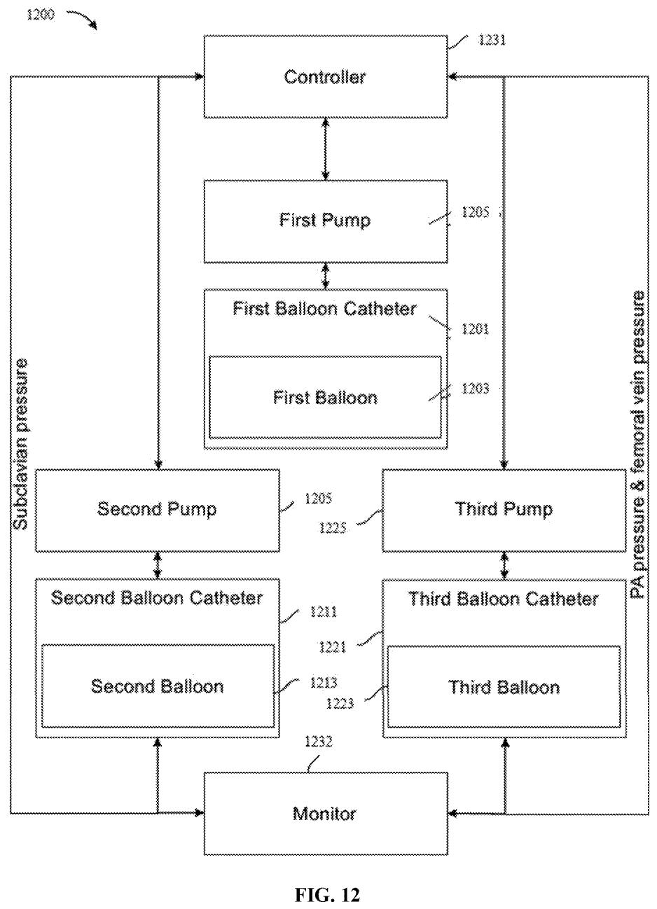

FIG. 12 is a schematic depicting the balloon catheter system 1200 for the treatment of heart failure, according to some embodiments where three balloons are utilized. The first balloon catheter 1201 may be mechanically connected to a first pump 1205 configured to inflate the first balloon 1203. The first pump 1205 may either be electronically or mechanically connected to a controller 1231 configured drive the first pump 1205. In some embodiments, a user selects inputs a desired inflation pressure for the first balloon 1203 into the controller 1231 which drives the first pump 1205 inflating the first balloon 1203.

The second balloon catheter 1211 may be mechanically connected to a second pump 1215 configured to inflate the second balloon 1213. The second pump 1215 may either be electronically or mechanically connected to the controller 1231 configured drive the second pump 1215. The second balloon catheter 1211 may either be electronically or mechanically connected to a monitor 1232 (e.g., pressure measuring device) configured to measure the subclavian pressure. In some embodiments, a user selects inputs a desired inflation pressure for the second balloon 1213 into the controller 1231 which drives the second pump 1215 inflating the second balloon 1213. The monitor 1232 may continuously or discretely measure the pressure at an area near the second balloon 1213 (e.g., the subclavian pressure). In some embodiments, a user may cause the second balloon 1213 to be deflated using the controller 1232 in response to an increase in the subclavian pressure. In some embodiments, the monitor 1232 may either be electronically or mechanically connected to the controller 1231. In some embodiments, the monitor 1232 is configured to transmit the subclavian pressure to the controller 1231. In some embodiments, the controller 1231 is configured to automatically cause the second balloon 1213 to be deflated in response to an increase in the subclavian pressure.

The third balloon catheter 1221 may be mechanically connected to a third pump 1225 configured to inflate the third balloon 1223. The third pump 1225 may either be electronically or mechanically connected to the controller 1231 configured drive the third pump 1225. The third balloon catheter 1221 may either be electronically or mechanically connected to a monitor 1232 (e.g., pressure measuring device) configured to measure the pulmonary artery (“PA”) and femoral vein pressures. In some embodiments, a user selects inputs a desired inflation pressure for the third balloon 1223 into the controller 1231 which drives the third pump 1225 inflating the third balloon 1223. The monitor 1232 may continuously or discretely measure the pressure at an area near the third balloon 1223 (e.g., the pulmonary artery pressure). The monitor 1232 may continuously or discretely measure the pressure at an area near the inlet of the first catheter 1201 (e.g., the femoral vein pressure). In some embodiments, a user may cause the third balloon 1223 to be deflated using the controller 1231 in response to an increase in with the pulmonary artery pressure or femoral vein pressure. In some embodiments, the monitor 1232 may either be electronically or mechanically connected to the controller 1231. In some embodiments, the monitor 1232 is configured to transmit the pulmonary artery and femoral vein pressures to the controller 1231. In some embodiments, the controller 1231 is configured to automatically cause the third balloon 1223 to be deflated in response to an increase in either the pulmonary artery or femoral vein pressures.

### Example 8: Exemplary Occlusion Method for Treatment of Heart Failure Using An Exemplary Balloon Catheter System

FIGS. 13A-13D are illustrations depicting a method for using the balloon catheter systems described herein to treat heart failure, according to some embodiments. FIG. 13A shows the step of introducing the balloon catheter system into the femoral vein through a puncture site. FIG. 13B shows the step of advancing the first balloon 1303 of the first balloon catheter 1301 to the first target location at the inferior vena cava (“IVC”) proximal to either the left or right renal veins. FIG. 13C shows the step of advancing the second balloon 1313 of the second balloon catheter 1311 to the second target location proximal to either the left or right subclavian vein. FIG. 13D shows the step of inflating the first balloon 1303 and second balloon 1313 of the balloon catheter system.

In some examples, the method may further comprises enlarging the puncture site using the detachable sheath and advancing the first balloon of the first balloon catheter through the detachable sheath to the first target location. In some examples, after the first balloon is positioned at the first target location, the detachable sheath may be removed, such as, for example, by tearing the detachable sheath using the first detachable arm and a second detachable arm.

In some examples, the first balloon may be advanced to the first target location using a guidewire. In some examples, the second balloon may be advanced to the second target location using a guidewire (“GW”).

In some examples, the first balloon may further comprise at least one radio marker allowing a user to visualize the first balloon under radiography or fluoroscopy, such that the user may accurately guide the first balloon to the first target location. In some examples, the second balloon may further comprise at least one radio marker allowing a user to visualize the second balloon under radiography or fluoroscopy, such that the user may accurately guide the second balloon to the second target location.

In some examples, intermittent occlusion of one or more veins may reduce venous blood backflow to the heart and/or reduces the pumping burden for the heart. In some embodiments, inflation of the first balloon may create a low-pressure area at or below the renal vein(s), improving blood and lymphatic return and organ function. In some embodiments, the low-pressure area in the renal vein may promote renal circulation, speed up urination, and/or reduce fluid retention in the body. In some examples, inflation of the first balloon may stimulate the vagus nerve causing enhanced blood accommodation in the lower extremities and abdomen. In some examples, stimulation of the vagus nerve can reduce venous blood backflow to the heart and reduce the pumping burden for the heart.

In some examples, the first balloon of the first balloon catheter may be positioned at a first target location in the inferior vena cava (“IVC”) proximal to either the left or right renal veins. In some examples, the first balloon may be positioned away from the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some examples, the first balloon may be positioned away from the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some examples, the first balloon may be from about 1 mm to about 25 mm away from the left or right renal vein. In some examples, the first balloon may be from about 5 mm to about 25 mm away from the left or right renal vein. In some examples, the first balloon may be from about 10 mm to about 25 mm away from the left or right renal vein. In some examples, the first balloon may be from about 15 mm to about 25 mm away from the left or right renal vein. In some examples, the first balloon may be from about 15 mm to about 20 mm away from the left or right renal vein.

In some examples, the first balloon may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some examples, the first balloon may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some examples, the first balloon may be inflated for a period from about 1 sec to about 60 sec. In some examples, the first balloon may be inflated for a period from about 10 sec to about 40 sec. In some examples, the first balloon may be inflated for a period from about 20 sec to about 40 sec. In some examples, inflation of the first balloon may occlude the inferior vena cava near the renal veins by about 10%, 20%, 30%, 40%, 50%, 60%, 70%, 80%, 90%, or any range therebetween.

In some examples, intermittent occlusion of one or more veins may reduce venous blood backflow to the heart and/or reduce the pumping burden for the heart. In some examples, inflation of the second balloon may create a low-pressure area near the left internal jugular vein and thoracic duct, improving return to the SVC and the right atrium of the heart, increases lymphatic reflow, and/or reduces fluid retention in the body. For example, blood pressure near the left internal jugular vein and thoracic duct, as measured by the second balloon catheter, may be reduced by about 5%, 10%, 15%, 20%, 25%, 30%, 35%, 40%, 45%, 50%, 55%, 60%, 65%, 70%, 75%, 80%, 85%, 90%, 95%, or any range therebetween with the second balloon inflated. In some examples, inflation of the second balloon may stimulate the vagus nerve, causing improved cardiac remodeling and function. In some examples, stimulation of the vagus nerve can cause vasodilation which may increase blood accommodation in the venous bed and reduces heart preload. In some examples, stimulation of the vagus nerve can reduce venous blood backflow to the heart and reduce the pumping burden for the heart.

In some examples, the second balloon may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some examples, the second balloon may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some examples, the second balloon may be inflated for a period from about 1 sec to about 60 sec. In some examples, the second balloon may be inflated for a period from about 10 sec to about 40 sec. In some examples, the first balloon may be inflated for a period from about 10 sec to about 30 sec. In some examples, inflation of the second balloon may occlude the subclavian vein by about 10%, 20%, 30%, 40%, 50%, 60%, 70%, 80%, 90%, or any range therebetween.

In some examples, the method may further comprise the step of measuring the pressure of at the subclavian vein using the second balloon catheter. In some examples, the measured pressure may be used to determine whether to inflate or deflate the first balloon and/or second balloon. In some examples, inflation or deflation of the first balloon may cause a pressure change (e.g., modulates the pressure) at or below the renal vein. For example, inflation of the first balloon may cause an increase in pressure below the renal vein. In some embodiments, when the pressure at the renal vein increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the first balloon is deflated. In some embodiments, when the pressure at the renal vein increases by about 1 mmHg to about 10 mmHg, the first balloon is deflated. In some embodiments, when the pressure at the renal vein increases by about 3 mmHg to about 5 mmHg, the first balloon is deflated.

Inflation or deflation of the second balloon may cause a pressure change (e.g., modulates the pressure) near the left internal jugular vein and thoracic duct. For example, inflation of the second balloon may cause an increase in pressure near the left internal jugular vein and thoracic duct. In some embodiments, when the pressure at the left internal jugular vein and thoracic duct increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the second balloon is deflated. In some embodiments, when the pressure at the left internal jugular vein and thoracic duct increases by about 1 mmHg to about 10 mmHg, the second balloon is deflated. In some embodiments, when the pressure at the left internal jugular vein and thoracic duct increases by about 5 mmHg to about 10 mmHg, the second balloon is deflated.

### Example 9: Exemplary Occlusion Method for Treatment of Heart Failure Using an Exemplary Balloon Catheter System

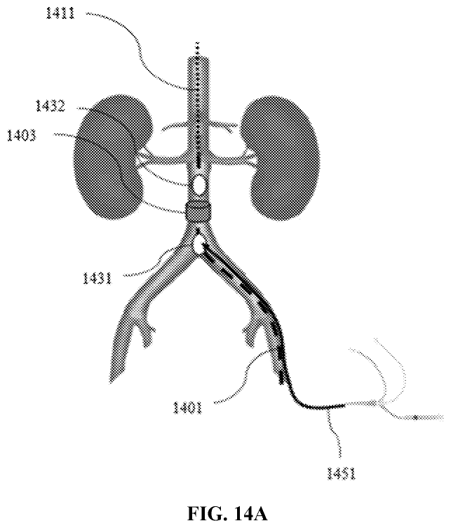

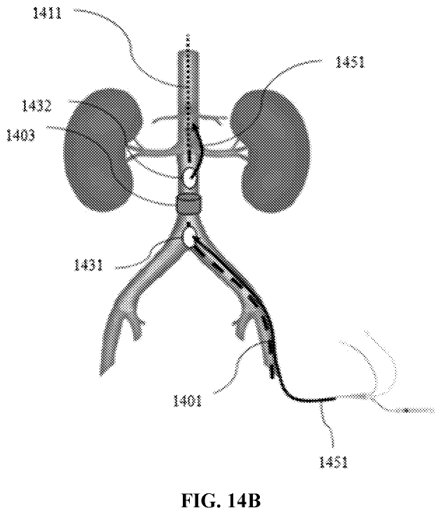

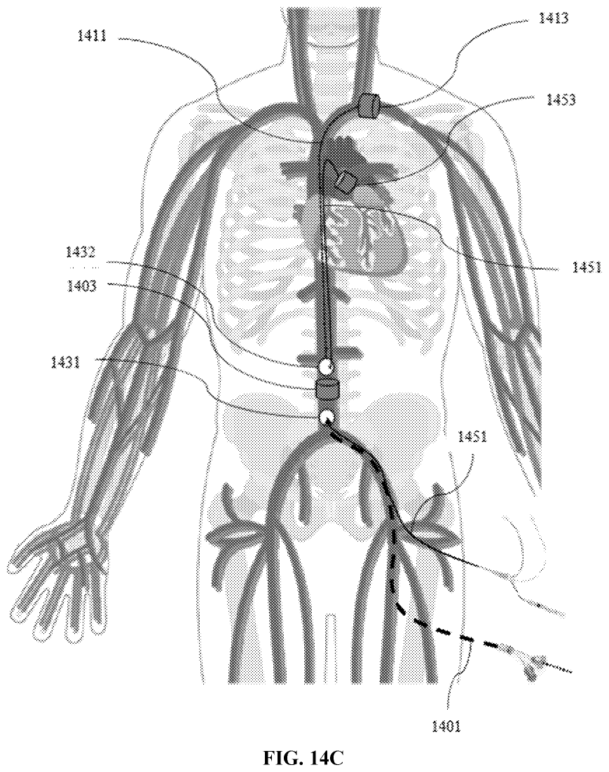

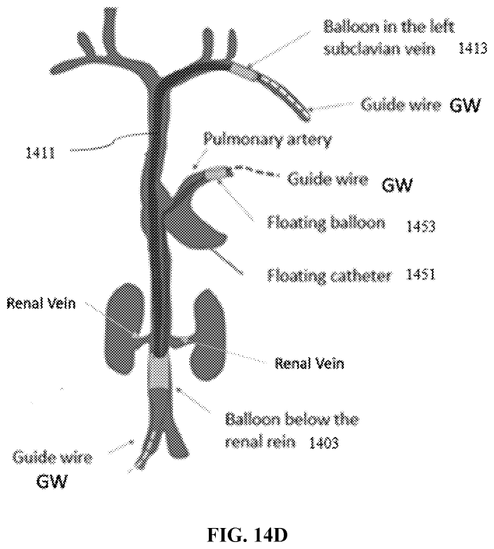

FIGS. 14A-14D are illustrations depicting a method for advancing a pulmonary artery catheter to the third target location at the pulmonary artery. FIG. 14A shows the step of introducing the third balloon catheter 1451 into the inlet 1431 of the first balloon catheter 1401. The third balloon catheter 1451 is advanced through the third lumen of the first balloon catheter 1401 until exiting through the outlet 1432 of the first balloon catheter 1401, as shown in FIG. 14B. FIG. 14C shows the step of advancing the third balloon of the third balloon catheter 1451 to the third target location proximal to the pulmonary artery. Once positioned at the third target location, the third balloon is inflated and the pressure at one or more of the pulmonary artery or right atria may be measured. FIG. 14D shows the final positioning of the balloon catheter system for use to treat heart failure.

In some examples, the third balloon is advanced to the third target location using a guidewire.

In some examples, the outlet 1432 of the first balloon catheter 1401 may be positioned in the inferior vena cava below the left or right renal veins. In some examples, the outlet 1432 may be positioned below the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some examples, the outlet 302 may be positioned below the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some examples, the outlet 1432 may be from about 1 mm to about 25 mm below the left or right renal vein. In some embodiments, the outlet 1432 may be from about 5 mm to about 20 mm below the left or right renal vein. In some examples, the outlet 1432 may be from about 5 mm to about 15 mm below the left or right renal vein. In some examples, the outlet 1432 is about 10 mm below the left or right renal vein.

In some examples, the third balloon 1453 comprises further at least one radio marker allowing a user to visualize the third balloon 1453 under radiography or fluoroscopy, such that the user may accurately guide the third balloon 1453 to the second target location.

### Example 10: Exemplary Occlusion Treatment of Acute Decompensated Heart Failure of a Subject

The devices and methods disclosed herein were utilized for human subjects. This test is intended to treat acute decompensated heart failure for feasibility, safety, and efficacy.

Subject selection was limited to individuals exhibiting the following: Class III-IV NYHA Classification, receiving another anti-ADHF treatment for at least 48 hours before occlusion treatment, consistent fluid overload, PAWP of more than 15 mmHg, and high level of NT-proBNP. Seven subjects were selected for treatment (7 Male/0 Female; Ages 44-76, ADHF diagnosis).

All subjects were subjected to the following phases: Screening, Treatment, 24 Hour Post-Op Observation, Discharge, 30 Day Post-Op Observation, and 90 Day Post-Op Observation. Screening consisted of demographic evaluation, echocardiogram, NT-proBNP testing, and medication. Treatment consisted of providing treatment, right heart catheterization (RHC), intra-aortic balloon pumping (IBP), adverse effect or serious adverse effect monitoring, and echocardiogram. During treatment, device implementation proceeded smoothly and the system operated normally, with heart rate and blood pressure remaining stable during treatment. Monitoring 24 hour Post-Op consisted of echocardiogram, blood tests, tests for thrombosis, tests for vessel injury, and adverse effect or serious adverse effect monitoring. Discharge consisted of echocardiogram, tests for thrombosis, tests for vessel injury, and adverse effect or serious adverse effect monitoring, and confirmation of life. Monitoring 30 days Post-op consisted of echocardiogram, NT-proBNP testing, determination of rehospitalization, medication, adverse effect or serious adverse effect monitoring, and confirmation of life. Monitoring 90 days Post-op consisted of echocardiogram, NT-proBNP testing, determination of rehospitalization, medication, adverse effect or serious adverse effect monitoring, and confirmation of life.

All subjects experienced stable blood pressure during treatment. No device related adverse effects or serious adverse effects resulted. One instance of injection site effects was experienced, exhibited as left upper arm hematoma. One patient experienced rehospitalization 30 days post-op.

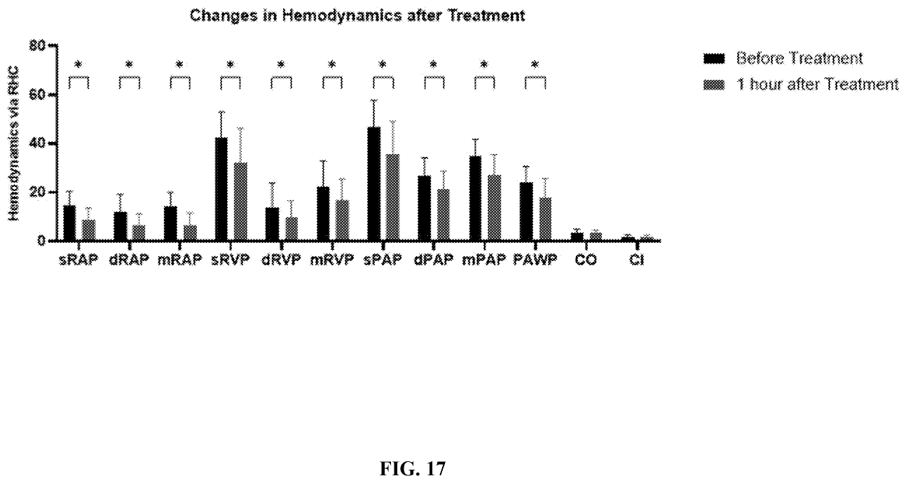

Subjects experienced improvement of cardiac preload after treatment, where improvement was exhibited by reduction of right atrial pressure (RAP) and pulmonary artery wedge pressure (PAWP) after treatment, among other things. Subjects experienced improvement of pulmonary circulation after treatment, where improvement was exhibited by reduction of pulmonary artery pressure (PAP) and right ventricular pressure (RVP), among other things. FIG. 17 shows the significant hemodynamic improvements from before treatment to 1 hour after treatment, including for sRAP (systolic right atrial pressure), dRAP (diastolic right atrial pressure), mRAP (mean right atrial pressure), sRVP (systolic right ventricular pressure), dRVP (diastolic right ventricular pressure), mRVP (mean right ventricular pressure), sPAP (systolic pulmonary artery pressure), dPAP (diastolic pulmonary artery pressure), mPAP (mean pulmonary artery pressure), PAWP (pulmonary artery wedge pressure), CO (cardiac output), and CI (cardiac index).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-80. (canceled)

81. A method for treating a cardiovascular disease of a subject, the method comprising:
inserting at least a portion of a catheter system into a femoral vein of the subject;
positioning a first balloon of the catheter system within the inferior vena cava of the subject at or near at least one renal vein;
positioning a second balloon of the catheter system at or near a subclavian vein of the subject; and
intermittently inflating the first balloon and the second balloon for a predetermined time period;
wherein the intermittent inflation of the second balloon creates a low-pressure area at one or more of the left internal jugular vein or the thoracic duct of the subject, and wherein the intermittent inflation of the first balloon creates a low-pressure area at the renal vein junction of the subject,
wherein the first balloon and second balloon are inflated simultaneously for a certain time of the pre-determined time period.

82. The method of claim 81, further comprising:
measuring the pressure at the subclavian vein of the subject using the catheter system; and
adjusting the inflation of one or more of the first balloon or second balloon in response to the measured subclavian vein pressure.

83. The method of claim 81, wherein adjusting the inflation of the first balloon modulates the pressure at the renal vein junction, and wherein adjusting the inflation of the second balloon modulates the pressure at the one or more of the left internal jugular vein or thoracic duct.

84. The method of claim 81, wherein inflation of the first balloon or second balloon stimulates a vagus nerve of the subject.

85. The method of claim 81, wherein inflating the first balloon comprises maintaining inflation of the first balloon for a portion of the pre-determined time before deflating the first balloon, wherein the pre-determined time is between from about 1 minute to about 30 minutes.

86. The method of claim 81, wherein inflating the second balloon comprises maintaining inflation of the second balloon for a portion of the pre-determined time before deflating the second balloon, wherein the pre-determined time is between from about 1 minute to about 30 minute.

87. The method of claim 81, wherein the first balloon and second balloon are simultaneously inflated for 20% to 100% of the pre-determined time.

88. The method of claim 81, wherein inflating the first balloon comprises maintaining inflation of the first balloon before deflating the first balloon.

89. The method of claim 81, wherein inflating the second balloon comprises maintaining inflation of the first balloon before deflating the second balloon.

90. The method of claim 81, wherein positioning the first balloon of the catheter system within the inferior vena cava comprises advancing a first catheter of the catheter system through vasculature of the subject over a guidewire.

91. The method of claim 81, wherein positioning the second balloon at or near the subclavian vein of the subject comprises advancing a second catheter of the catheter system through vasculature of the subject over a guidewire.

92. The method of claim 81, further comprising:
inserting a catheter further comprising a third balloon into a lumen of the catheter system;
positioning the third balloon of the catheter at or near the pulmonary artery;
inflating the third balloon; and
measuring a pulmonary artery pressure of the subject.

93. The method of claim 92, further comprising monitoring at least one hemodynamic parameter of the subject with the catheter.

94. The method of claim 93, wherein the at least one hemodynamic parameter is one or more of heart rate, blood pressure, stroke volume, cardiac output, or total peripheral resistance.

95. The method of claim 81, further comprising monitoring pressure at a vein of the subject with the catheter system.

96. The method of claim 81, further comprising monitoring pressure at one or more of an inferior vena cava or femoral vein with the catheter system.

97. The method of claim 81, wherein the cardiovascular disease is heart failure or acute heart failure.

98. The method of claim 81, wherein the intermittent inflation of the first balloon promotes renal perfusion, promotes diuresis, reduces fluid retention, or decreases venous return to the heart.

99. The method of claim 81, wherein the intermittent inflation of the second balloon enhances venous drainage, enhances lymphatic drainage, relieves fluid overload, or relieves venous congestion.

100. The method of claim 81, wherein the intermittent inflation of the first and second balloon reduces pulmonary artery pressure.
